SEQUENCE LISTING (970 sequences)

SEQ ID NO: 1 - protein, 441 aa, Artificial Sequence
  Synthetic peptide
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 7 aa, Artificial Sequence
  Synthetic peptide
QIVYKPV

SEQ ID NO: 3 - protein, 6 aa, Artificial Sequence
  Synthetic peptide
QIVYKP

SEQ ID NO: 4 - protein, 6 aa, Artificial Sequence
  Synthetic peptide
NIKHVP

SEQ ID NO: 5 - protein, 7 aa, Artificial Sequence
  Synthetic peptide
NIKHVPG

SEQ ID NO: 6 - protein, 6 aa, Artificial Sequence
  Synthetic peptide
HVPGGG

SEQ ID NO: 7 - protein, 5 aa, Artificial Sequence
  Synthetic peptide
HVPGG

SEQ ID NO: 8 - protein, 6 aa, Artificial Sequence
  Synthetic peptide
HKPGGG

SEQ ID NO: 9 - protein, 5 aa, Artificial Sequence
  Synthetic peptide
HKPGG

SEQ ID NO: 10 - protein, 7 aa, Artificial Sequence
  Synthetic peptide
KHVPGGG

SEQ ID NO: 11 - protein, 6 aa, Artificial Sequence
  Synthetic peptide
KHVPGG

SEQ ID NO: 12 - protein, 6 aa, Artificial Sequence
  Synthetic peptide
HQPGGG

SEQ ID NO: 13 - protein, 5 aa, Artificial Sequence
  Synthetic peptide
HQPGG

SEQ ID NO: 14 - protein, 6 aa, Artificial Sequence
  Synthetic peptide
VQIINK

SEQ ID NO: 15 - protein, 7 aa, Artificial Sequence
  Synthetic peptide
VQIINKK

SEQ ID NO: 16 - protein, 8 aa, Artificial Sequence
  Synthetic peptide
VQIINKKL

SEQ ID NO: 17 - protein, 5 aa, Artificial Sequence
  Synthetic peptide
QIINK

SEQ ID NO: 18 - protein, 6 aa, Artificial Sequence
  Synthetic peptide
QIINKK

SEQ ID NO: 19 - protein, 7 aa, Artificial Sequence
  Synthetic peptide
QIINKKL

SEQ ID NO: 20 - protein, 7 aa, Artificial Sequence
  Synthetic peptide
QIVYKSV

SEQ ID NO: 21 - protein, 7 aa, Artificial Sequence
  Synthetic peptide
EIVYKSV

SEQ ID NO: 22 - protein, 7 aa, Artificial Sequence
  Synthetic peptide
EIVYKPV

SEQ ID NO: 23 - protein, 7 aa, Artificial Sequence
  Synthetic peptide
CNIKHVP

SEQ ID NO: 24 - protein, 8 aa, Artificial Sequence
  Synthetic peptide
CNIKHVPG

SEQ ID NO: 25 - protein, 7 aa, Artificial Sequence
  Synthetic peptide
EIVYKSP

SEQ ID NO: 26 - protein, 7 aa, Artificial Sequence
  Synthetic peptide
IVYKSPV

SEQ ID NO: 27 - protein, 4 aa, Artificial Sequence
  Synthetic peptide
IVYK

SEQ ID NO: 28 - protein, 10 aa, Artificial Sequence
  Synthetic peptide
VPGGGSVQIV

SEQ ID NO: 29 - protein, 9 aa, Artificial Sequence
  Synthetic peptide
PGGGSVQIV

SEQ ID NO: 30 - protein, 8 aa, Artificial Sequence
  Synthetic peptide
GGGSVQIV

SEQ ID NO: 31 - protein, 7 aa, Artificial Sequence
  Synthetic peptide
GGSVQIV

SEQ ID NO: 32 - protein, 6 aa, Artificial Sequence
  Synthetic peptide
GSVQIV

SEQ ID NO: 33 - protein, 5 aa, Artificial Sequence
  Synthetic peptide
SVQIV

SEQ ID NO: 34 - protein, 4 aa, Artificial Sequence
  Synthetic peptide
VQIV

SEQ ID NO: 35 - protein, 3 aa, Artificial Sequence
  Synthetic peptide
QIV

SEQ ID NO: 36 - protein, 10 aa, Artificial Sequence
  Synthetic peptide
PGGGSVQIVY

SEQ ID NO: 37 - protein, 9 aa, Artificial Sequence
  Synthetic peptide
GGGSVQIVY

SEQ ID NO: 38 - protein, 8 aa, Artificial Sequence
  Synthetic peptide
GGSVQIVY

SEQ ID NO: 39 - protein, 7 aa, Artificial Sequence
  Synthetic peptide
GSVQIVY

SEQ ID NO: 40 - protein, 6 aa, Artificial Sequence
  Synthetic peptide
SVQIVY

SEQ ID NO: 41 - protein, 5 aa, Artificial Sequence
  Synthetic peptide
VQIVY

SEQ ID NO: 42 - protein, 4 aa, Artificial Sequence
  Synthetic peptide
QIVY

SEQ ID NO: 43 - protein, 3 aa, Artificial Sequence
  Synthetic peptide
IVY

SEQ ID NO: 44 - protein, 10 aa, Artificial Sequence
  Synthetic peptide
GGGSVQIVYK

SEQ ID NO: 45 - protein, 9 aa, Artificial Sequence
  Synthetic peptide
GGSVQIVYK

SEQ ID NO: 46 - protein, 8 aa, Artificial Sequence
  Synthetic peptide
GSVQIVYK

SEQ ID NO: 47 - protein, 7 aa, Artificial Sequence
  Synthetic peptide
SVQIVYK

SEQ ID NO: 48 - protein, 6 aa, Artificial Sequence
  Synthetic peptide
VQIVYK

SEQ ID NO: 49 - protein, 5 aa, Artificial Sequence
  Synthetic peptide
QIVYK

SEQ ID NO: 50 - protein, 3 aa, Artificial Sequence
  Synthetic peptide
VYK

SEQ ID NO: 51 - protein, 10 aa, Artificial Sequence
  Synthetic peptide
GGSVQIVYKP

SEQ ID NO: 52 - protein, 9 aa, Artificial Sequence
  Synthetic peptide
GSVQIVYKP

SEQ ID NO: 53 - protein, 8 aa, Artificial Sequence
  Synthetic peptide
SVQIVYKP

SEQ ID NO: 54 - protein, 7 aa, Artificial Sequence
  Synthetic peptide
VQIVYKP

SEQ ID NO: 55 - protein, 5 aa, Artificial Sequence
  Synthetic peptide
IVYKP

SEQ ID NO: 56 - protein, 4 aa, Artificial Sequence
  Synthetic peptide
VYKP

SEQ ID NO: 57 - protein, 3 aa, Artificial Sequence
  Synthetic peptide
YKP

SEQ ID NO: 58 - protein, 10 aa, Artificial Sequence
  Synthetic peptide
GSVQIVYKPV

SEQ ID NO: 59 - protein, 9 aa, Artificial Sequence
  Synthetic peptide
SVQIVYKPV

SEQ ID NO: 60 - protein, 8 aa, Artificial Sequence
  Synthetic peptide
VQIVYKPV

SEQ ID NO: 61 - protein, 6 aa, Artificial Sequence
  Synthetic peptide
IVYKPV

SEQ ID NO: 62 - protein, 5 aa, Artificial Sequence
  Synthetic peptide
VYKPV

SEQ ID NO: 63 - protein, 4 aa, Artificial Sequence
  Synthetic peptide
YKPV

SEQ ID NO: 64 - protein, 3 aa, Artificial Sequence
  Synthetic peptide
KPV

SEQ ID NO: 65 - protein, 10 aa, Artificial Sequence
  Synthetic peptide
SVQIVYKPVD

SEQ ID NO: 66 - protein, 9 aa, Artificial Sequence
  Synthetic peptide
VQIVYKPVD

SEQ ID NO: 67 - protein, 8 aa, Artificial Sequence
  Synthetic peptide
QIVYKPVD

SEQ ID NO: 68 - protein, 7 aa, Artificial Sequence
  Synthetic peptide
IVYKPVD

SEQ ID NO: 69 - protein, 6 aa, Artificial Sequence
  Synthetic peptide
VYKPVD

SEQ ID NO: 70 - protein, 5 aa, Artificial Sequence
  Synthetic peptide
YKPVD

SEQ ID NO: 71 - protein, 4 aa, Artificial Sequence
  Synthetic peptide
KPVD

SEQ ID NO: 72 - protein, 3 aa, Artificial Sequence
  Synthetic peptide
PVD

SEQ ID NO: 73 - protein, 10 aa, Artificial Sequence
  Synthetic peptide
VQIVYKPVDL

SEQ ID NO: 74 - protein, 9 aa, Artificial Sequence
  Synthetic peptide
QIVYKPVDL

SEQ ID NO: 75 - protein, 8 aa, Artificial Sequence
  Synthetic peptide
IVYKPVDL

SEQ ID NO: 76 - protein, 7 aa, Artificial Sequence
  Synthetic peptide
VYKPVDL

SEQ ID NO: 77 - protein, 6 aa, Artificial Sequence
  Synthetic peptide
YKPVDL

SEQ ID NO: 78 - protein, 5 aa, Artificial Sequence
  Synthetic peptide
KPVDL

SEQ ID NO: 79 - protein, 4 aa, Artificial Sequence
  Synthetic peptide
PVDL

SEQ ID NO: 80 - protein, 3 aa, Artificial Sequence
  Synthetic peptide
VDL

SEQ ID NO: 81 - protein, 10 aa, Artificial Sequence
  Synthetic peptide
QIVYKPVDLS

SEQ ID NO: 82 - protein, 9 aa, Artificial Sequence
  Synthetic peptide
IVYKPVDLS

SEQ ID NO: 83 - protein, 8 aa, Artificial Sequence
  Synthetic peptide
VYKPVDLS

SEQ ID NO: 84 - protein, 7 aa, Artificial Sequence
  Synthetic peptide
YKPVDLS

SEQ ID NO: 85 - protein, 6 aa, Artificial Sequence
  Synthetic peptide
KPVDLS

SEQ ID NO: 86 - protein, 5 aa, Artificial Sequence
  Synthetic peptide
PVDLS

SEQ ID NO: 87 - protein, 4 aa, Artificial Sequence
  Synthetic peptide
VDLS

SEQ ID NO: 88 - protein, 10 aa, Artificial Sequence
  Synthetic peptide
IVYKPVDLSK

SEQ ID NO: 89 - protein, 9 aa, Artificial Sequence
  Synthetic peptide
VYKPVDLSK

SEQ ID NO: 90 - protein, 8 aa, Artificial Sequence
  Synthetic peptide
YKPVDLSK

SEQ ID NO: 91 - protein, 7 aa, Artificial Sequence
  Synthetic peptide
KPVDLSK

SEQ ID NO: 92 - protein, 6 aa, Artificial Sequence
  Synthetic peptide
PVDLSK

SEQ ID NO: 93 - protein, 5 aa, Artificial Sequence
  Synthetic peptide
VDLSK

SEQ ID NO: 94 - protein, 10 aa, Artificial Sequence
  Synthetic peptide
VYKPVDLSKV

SEQ ID NO: 95 - protein, 9 aa, Artificial Sequence
  Synthetic peptide
YKPVDLSKV

SEQ ID NO: 96 - protein, 8 aa, Artificial Sequence
  Synthetic peptide
KPVDLSKV

SEQ ID NO: 97 - protein, 7 aa, Artificial Sequence
  Synthetic peptide
PVDLSKV

SEQ ID NO: 98 - protein, 6 aa, Artificial Sequence
  Synthetic peptide
VDLSKV

SEQ ID NO: 99 - protein, 10 aa, Artificial Sequence
  Synthetic peptide
YKPVDLSKVT

SEQ ID NO: 100 - protein, 9 aa, Artificial Sequence
  Synthetic peptide
KPVDLSKVT

SEQ ID NO: 101 - protein, 8 aa, Artificial Sequence
  Synthetic peptide
PVDLSKVT

SEQ ID NO: 102 - protein, 7 aa, Artificial Sequence
  Synthetic peptide
VDLSKVT

SEQ ID NO: 103 - protein, 10 aa, Artificial Sequence
  Synthetic peptide
AKTDHGAEIV

SEQ ID NO: 104 - protein, 9 aa, Artificial Sequence
  Synthetic peptide
KTDHGAEIV

SEQ ID NO: 105 - protein, 8 aa, Artificial Sequence
  Synthetic peptide
TDHGAEIV

SEQ ID NO: 106 - protein, 7 aa, Artificial Sequence
  Synthetic peptide
DHGAEIV

SEQ ID NO: 107 - protein, 6 aa, Artificial Sequence
  Synthetic peptide
HGAEIV

SEQ ID NO: 108 - protein, 5 aa, Artificial Sequence
  Synthetic peptide
GAEIV

SEQ ID NO: 109 - protein, 4 aa, Artificial Sequence
  Synthetic peptide
AEIV

SEQ ID NO: 110 - protein, 3 aa, Artificial Sequence [sequence omitted]

SEQ ID NO: 111 - protein, 10 aa, Artificial Sequence
  Synthetic peptide
KTDHGAEIVY

SEQ ID NO: 112 - protein, 9 aa, Artificial Sequence
  Synthetic peptide
TDHGAEIVY

SEQ ID NO: 113 - protein, 8 aa, Artificial Sequence
  Synthetic peptide
DHGAEIVY

SEQ ID NO: 114 - protein, 7 aa, Artificial Sequence
  Synthetic peptide
HGAEIVY

SEQ ID NO: 115 - protein, 6 aa, Artificial Sequence
  Synthetic peptide
GAEIVY

SEQ ID NO: 116 - protein, 5 aa, Artificial Sequence
  Synthetic peptide
AEIVY

SEQ ID NO: 117 - protein, 4 aa, Artificial Sequence
  Synthetic peptide
EIVY

SEQ ID NO: 118 - protein, 10 aa, Artificial Sequence
  Synthetic peptide
TDHGAEIVYK

SEQ ID NO: 119 - protein, 9 aa, Artificial Sequence
  Synthetic peptide
DHGAEIVYK

SEQ ID NO: 120 - protein, 8 aa, Artificial Sequence
  Synthetic peptide
HGAEIVYK

SEQ ID NO: 121 - protein, 7 aa, Artificial Sequence
  Synthetic peptide
GAEIVYK

SEQ ID NO: 122 - protein, 6 aa, Artificial Sequence
  Synthetic peptide
AEIVYK

SEQ ID NO: 123 - protein, 5 aa, Artificial Sequence
  Synthetic peptide
EIVYK

SEQ ID NO: 124 - protein, 10 aa, Artificial Sequence
  Synthetic peptide
DHGAEIVYKS

SEQ ID NO: 125 - protein, 9 aa, Artificial Sequence
  Synthetic peptide
HGAEIVYKS

SEQ ID NO: 126 - protein, 8 aa, Artificial Sequence
  Synthetic peptide
GAEIVYKS

SEQ ID NO: 127 - protein, 7 aa, Artificial Sequence
  Synthetic peptide
AEIVYKS

SEQ ID NO: 128 - protein, 6 aa, Artificial Sequence
  Synthetic peptide
EIVYKS

SEQ ID NO: 129 - protein, 5 aa, Artificial Sequence
  Synthetic peptide
IVYKS

SEQ ID NO: 130 - protein, 4 aa, Artificial Sequence
  Synthetic peptide
VYKS

SEQ ID NO: 131 - protein, 3 aa, Artificial Sequence
  Synthetic peptide
YKS

SEQ ID NO: 132 - protein, 10 aa, Artificial Sequence
  Synthetic peptide
HGAEIVYKSP

SEQ ID NO: 133 - protein, 9 aa, Artificial Sequence
  Synthetic peptide
GAEIVYKSP

SEQ ID NO: 134 - protein, 8 aa, Artificial Sequence
  Synthetic peptide
AEIVYKSP

SEQ ID NO: 135 - protein, 6 aa, Artificial Sequence
  Synthetic peptide
IVYKSP

SEQ ID NO: 136 - protein, 5 aa, Artificial Sequence
  Synthetic peptide
VYKSP

SEQ ID NO: 137 - protein, 4 aa, Artificial Sequence
  Synthetic peptide
YKSP

SEQ ID NO: 138 - protein, 3 aa, Artificial Sequence
  Synthetic peptide
KSP

SEQ ID NO: 139 - protein, 10 aa, Artificial Sequence
  Synthetic peptide
GAEIVYKSPV

SEQ ID NO: 140 - protein, 9 aa, Artificial Sequence
  Synthetic peptide
AEIVYKSPV

SEQ ID NO: 141 - protein, 8 aa, Artificial Sequence
  Synthetic peptide
EIVYKSPV

SEQ ID NO: 142 - protein, 6 aa, Artificial Sequence
  Synthetic peptide
VYKSPV

SEQ ID NO: 143 - protein, 5 aa, Artificial Sequence
  Synthetic peptide
YKSPV

SEQ ID NO: 144 - protein, 4 aa, Artificial Sequence
  Synthetic peptide
KSPV

SEQ ID NO: 145 - protein, 3 aa, Artificial Sequence
  Synthetic peptide
SPV

SEQ ID NO: 146 - protein, 10 aa, Artificial Sequence
  Synthetic peptide
AEIVYKSPVV

SEQ ID NO: 147 - protein, 9 aa, Artificial Sequence
  Synthetic peptide
EIVYKSPVV

SEQ ID NO: 148 - protein, 8 aa, Artificial Sequence
  Synthetic peptide
IVYKSPVV

SEQ ID NO: 149 - protein, 7 aa, Artificial Sequence
  Synthetic peptide
VYKSPVV

SEQ ID NO: 150 - protein, 6 aa, Artificial Sequence
  Synthetic peptide
YKSPVV

SEQ ID NO: 151 - protein, 5 aa, Artificial Sequence
  Synthetic peptide
KSPVV

SEQ ID NO: 152 - protein, 4 aa, Artificial Sequence
  Synthetic peptide
SPVV

SEQ ID NO: 153 - protein, 3 aa, Artificial Sequence
  Synthetic peptide
PVV

SEQ ID NO: 154 - protein, 10 aa, Artificial Sequence
  Synthetic peptide
EIVYKSPVVS

SEQ ID NO: 155 - protein, 9 aa, Artificial Sequence
  Synthetic peptide
IVYKSPVVS

SEQ ID NO: 156 - protein, 8 aa, Artificial Sequence
  Synthetic peptide
VYKSPVVS

SEQ ID NO: 157 - protein, 7 aa, Artificial Sequence
  Synthetic peptide
YKSPVVS

SEQ ID NO: 158 - protein, 6 aa, Artificial Sequence
  Synthetic peptide
KSPVVS

SEQ ID NO: 159 - protein, 5 aa, Artificial Sequence
  Synthetic peptide
SPVVS

SEQ ID NO: 160 - protein, 3 aa, Artificial Sequence
  Synthetic peptide
VVS

SEQ ID NO: 161 - protein, 10 aa, Artificial Sequence
  Synthetic peptide
IVYKSPVVSG

SEQ ID NO: 162 - protein, 9 aa, Artificial Sequence
  Synthetic peptide
VYKSPVVSG

SEQ ID NO: 163 - protein, 8 aa, Artificial Sequence
  Synthetic peptide
YKSPVVSG

SEQ ID NO: 164 - protein, 7 aa, Artificial Sequence
  Synthetic peptide
KSPVVSG

SEQ ID NO: 165 - protein, 6 aa, Artificial Sequence
  Synthetic peptide
SPVVSG

SEQ ID NO: 166 - protein, 5 aa, Artificial Sequence
  Synthetic peptide
PVVSG

SEQ ID NO: 167 - protein, 10 aa, Artificial Sequence
  Synthetic peptide
VYKSPVVSGD

SEQ ID NO: 168 - protein, 9 aa, Artificial Sequence
  Synthetic peptide
YKSPVVSGD

SEQ ID NO: 169 - protein, 8 aa, Artificial Sequence
  Synthetic peptide
KSPVVSGD

SEQ ID NO: 170 - protein, 7 aa, Artificial Sequence
  Synthetic peptide
SPVVSGD

SEQ ID NO: 171 - protein, 6 aa, Artificial Sequence
  Synthetic peptide
PVVSGD

SEQ ID NO: 172 - protein, 5 aa, Artificial Sequence
  Synthetic peptide
VVSGD

SEQ ID NO: 173 - protein, 10 aa, Artificial Sequence
  Synthetic peptide
YKSPVVSGDT

SEQ ID NO: 174 - protein, 9 aa, Artificial Sequence
  Synthetic peptide
KSPVVSGDT

SEQ ID NO: 175 - protein, 8 aa, Artificial Sequence
  Synthetic peptide
SPVVSGDT

SEQ ID NO: 176 - protein, 7 aa, Artificial Sequence
  Synthetic peptide
PVVSGDT

SEQ ID NO: 177 - protein, 10 aa, Artificial Sequence
  Synthetic peptide
KSPVVSGDTS

SEQ ID NO: 178 - protein, 9 aa, Artificial Sequence
  Synthetic peptide
SPVVSGDTS

SEQ ID NO: 179 - protein, 8 aa, Artificial Sequence
  Synthetic peptide
PVVSGDTS

SEQ ID NO: 180 - protein, 7 aa, Artificial Sequence
  Synthetic peptide
VVSGDTS

SEQ ID NO: 181 - protein, 10 aa, Artificial Sequence
  Synthetic peptide
SPVVSGDTSP

SEQ ID NO: 182 - protein, 9 aa, Artificial Sequence
  Synthetic peptide
PVVSGDTSP

SEQ ID NO: 183 - protein, 8 aa, Artificial Sequence
  Synthetic peptide
VVSGDTSP

SEQ ID NO: 184 - protein, 10 aa, Artificial Sequence
  Synthetic peptide
PVVSGDTSPR

SEQ ID NO: 185 - protein, 10 aa, Artificial Sequence
  Synthetic peptide
HQPGGGKVQI

SEQ ID NO: 186 - protein, 9 aa, Artificial Sequence
  Synthetic peptide
QPGGGKVQI

SEQ ID NO: 187 - protein, 8 aa, Artificial Sequence
  Synthetic peptide
PGGGKVQI

SEQ ID NO: 188 - protein, 7 aa, Artificial Sequence
  Synthetic peptide
GGGKVQI

SEQ ID NO: 189 - protein, 6 aa, Artificial Sequence
  Synthetic peptide
GGKVQI

SEQ ID NO: 190 - protein, 5 aa, Artificial Sequence
  Synthetic peptide
GKVQI

SEQ ID NO: 191 - protein, 4 aa, Artificial Sequence
  Synthetic peptide
KVQI

SEQ ID NO: 192 - protein, 3 aa, Artificial Sequence
  Synthetic peptide
VQI

SEQ ID NO: 193 - protein, 10 aa, Artificial Sequence
  Synthetic peptide
QPGGGKVQII

SEQ ID NO: 194 - protein, 9 aa, Artificial Sequence
  Synthetic peptide
PGGGKVQII

SEQ ID NO: 195 - protein, 9 aa, Artificial Sequence
  Synthetic peptide
PGGGKVQII

SEQ ID NO: 196 - protein, 8 aa, Artificial Sequence
  Synthetic peptide
GGGKVQII

SEQ ID NO: 197 - protein, 7 aa, Artificial Sequence
  Synthetic peptide
GGKVQII

SEQ ID NO: 198 - protein, 6 aa, Artificial Sequence
  Synthetic peptide
GKVQII

SEQ ID NO: 199 - protein, 5 aa, Artificial Sequence
  Synthetic peptide
KVQII

SEQ ID NO: 200 - protein, 4 aa, Artificial Sequence
  Synthetic peptide
VQII

SEQ ID NO: 201 - protein, 3 aa, Artificial Sequence
  Synthetic peptide
QII

SEQ ID NO: 202 - protein, 10 aa, Artificial Sequence
  Synthetic peptide
PGGGKVQIIN

SEQ ID NO: 203 - protein, 9 aa, Artificial Sequence
  Synthetic peptide
GGGKVQIIN

SEQ ID NO: 204 - protein, 8 aa, Artificial Sequence
  Synthetic peptide
GGKVQIIN

SEQ ID NO: 205 - protein, 7 aa, Artificial Sequence
  Synthetic peptide
GKVQIIN

SEQ ID NO: 206 - protein, 6 aa, Artificial Sequence
  Synthetic peptide
KVQIIN

SEQ ID NO: 207 - protein, 5 aa, Artificial Sequence
  Synthetic peptide
VQIIN

SEQ ID NO: 208 - protein, 4 aa, Artificial Sequence
  Synthetic peptide
QIIN

SEQ ID NO: 209 - protein, 3 aa, Artificial Sequence
  Synthetic peptide
IIN

SEQ ID NO: 210 - protein, 10 aa, Artificial Sequence
  Synthetic peptide
GGGKVQIINK

SEQ ID NO: 211 - protein, 9 aa, Artificial Sequence
  Synthetic peptide
GGKVQIINK

SEQ ID NO: 212 - protein, 8 aa, Artificial Sequence
  Synthetic peptide
GKVQIINK

SEQ ID NO: 213 - protein, 7 aa, Artificial Sequence
  Synthetic peptide
KVQIINK

SEQ ID NO: 214 - protein, 4 aa, Artificial Sequence
  Synthetic peptide
IINK

SEQ ID NO: 215 - protein, 3 aa, Artificial Sequence
  Synthetic peptide
INK

SEQ ID NO: 216 - protein, 10 aa, Artificial Sequence
  Synthetic peptide
GGKVQIINKK

SEQ ID NO: 217 - protein, 9 aa, Artificial Sequence
  Synthetic peptide
GKVQIINKK

SEQ ID NO: 218 - protein, 8 aa, Artificial Sequence
  Synthetic peptide
KVQIINKK

SEQ ID NO: 219 - protein, 5 aa, Artificial Sequence
  Synthetic peptide
IINKK

SEQ ID NO: 220 - protein, 4 aa, Artificial Sequence
  Synthetic peptide
INKK

SEQ ID NO: 221 - protein, 3 aa, Artificial Sequence
  Synthetic peptide
NKK

SEQ ID NO: 222 - protein, 10 aa, Artificial Sequence
  Synthetic peptide
GKVQIINKKL

SEQ ID NO: 223 - protein, 9 aa, Artificial Sequence
  Synthetic peptide
KVQIINKKL

SEQ ID NO: 224 - protein, 6 aa, Artificial Sequence
  Synthetic peptide
IINKKL

SEQ ID NO: 225 - protein, 5 aa, Artificial Sequence
  Synthetic peptide
INKKL

SEQ ID NO: 226 - protein, 4 aa, Artificial Sequence
  Synthetic peptide
NKKL

SEQ ID NO: 227 - protein, 3 aa, Artificial Sequence
  Synthetic peptide
KKL

SEQ ID NO: 228 - protein, 10 aa, Artificial Sequence
  Synthetic peptide
KVQIINKKLD

SEQ ID NO: 229 - protein, 9 aa, Artificial Sequence
  Synthetic peptide
VQIINKKLD

SEQ ID NO: 230 - protein, 8 aa, Artificial Sequence
  Synthetic peptide
QIINKKLD

SEQ ID NO: 231 - protein, 7 aa, Artificial Sequence
  Synthetic peptide
IINKKLD

SEQ ID NO: 232 - protein, 6 aa, Artificial Sequence
  Synthetic peptide
INKKLD

SEQ ID NO: 233 - protein, 5 aa, Artificial Sequence
  Synthetic peptide
NKKLD

SEQ ID NO: 234 - protein, 4 aa, Artificial Sequence
  Synthetic peptide
KKLD

SEQ ID NO: 235 - protein, 10 aa, Artificial Sequence
  Synthetic peptide
VQIINKKLDL

SEQ ID NO: 236 - protein, 9 aa, Artificial Sequence
  Synthetic peptide
QIINKKLDL

SEQ ID NO: 237 - protein, 8 aa, Artificial Sequence
  Synthetic peptide
IINKKLDL

SEQ ID NO: 238 - protein, 7 aa, Artificial Sequence
  Synthetic peptide
INKKLDL

SEQ ID NO: 239 - protein, 6 aa, Artificial Sequence
  Synthetic peptide
NKKLDL

SEQ ID NO: 240 - protein, 5 aa, Artificial Sequence
  Synthetic peptide
KKLDL

SEQ ID NO: 241 - protein, 10 aa, Artificial Sequence
  Synthetic peptide
QIINKKLDLS

SEQ ID NO: 242 - protein, 9 aa, Artificial Sequence
  Synthetic peptide
IINKKLDLS

SEQ ID NO: 243 - protein, 8 aa, Artificial Sequence
  Synthetic peptide
INKKLDLS

SEQ ID NO: 244 - protein, 7 aa, Artificial Sequence
  Synthetic peptide
NKKLDLS

SEQ ID NO: 245 - protein, 6 aa, Artificial Sequence
  Synthetic peptide
KKLDLS

SEQ ID NO: 246 - protein, 10 aa, Artificial Sequence
  Synthetic peptide
IINKKLDLSN

SEQ ID NO: 247 - protein, 9 aa, Artificial Sequence
  Synthetic peptide
INKKLDLSN

SEQ ID NO: 248 - protein, 8 aa, Artificial Sequence
  Synthetic peptide
NKKLDLSN

SEQ ID NO: 249 - protein, 7 aa, Artificial Sequence
  Synthetic peptide
KKLDLSN

SEQ ID NO: 250 - protein, 10 aa, Artificial Sequence
  Synthetic peptide
INKKLDLSNV

SEQ ID NO: 251 - protein, 9 aa, Artificial Sequence
  Synthetic peptide
NKKLDLSNV

SEQ ID NO: 252 - protein, 8 aa, Artificial Sequence
  Synthetic peptide
KKLDLSNV

SEQ ID NO: 253 - protein, 10 aa, Artificial Sequence
  Synthetic peptide
NKKLDLSNVQ

SEQ ID NO: 254 - protein, 9 aa, Artificial Sequence
  Synthetic peptide
KKLDLSNVQ

SEQ ID NO: 255 - protein, 10 aa, Artificial Sequence
  Synthetic peptide
KKLDLSNVQS

SEQ ID NO: 256 - protein, 10 aa, Artificial Sequence
  Synthetic peptide
SKCGSKDNIK

SEQ ID NO: 257 - protein, 9 aa, Artificial Sequence
  Synthetic peptide
KCGSKDNIK

SEQ ID NO: 258 - protein, 8 aa, Artificial Sequence
  Synthetic peptide
CGSKDNIK

SEQ ID NO: 259 - protein, 6 aa, Artificial Sequence
  Synthetic peptide
SKDNIK

SEQ ID NO: 260 - protein, 5 aa, Artificial Sequence
  Synthetic peptide
KDNIK

SEQ ID NO: 261 - protein, 4 aa, Artificial Sequence
  Synthetic peptide
DNIK

SEQ ID NO: 262 - protein, 3 aa, Artificial Sequence
  Synthetic peptide
NIK

SEQ ID NO: 263 - protein, 10 aa, Artificial Sequence
  Synthetic peptide
KCGSKDNIKH

SEQ ID NO: 264 - protein, 9 aa, Artificial Sequence
  Synthetic peptide
CGSKDNIKH

SEQ ID NO: 265 - protein, 7 aa, Artificial Sequence
  Synthetic peptide
SKDNIKH

SEQ ID NO: 266 - protein, 6 aa, Artificial Sequence
  Synthetic peptide
KDNIKH

SEQ ID NO: 267 - protein, 5 aa, Artificial Sequence
  Synthetic peptide
DNIKH

SEQ ID NO: 268 - protein, 4 aa, Artificial Sequence
  Synthetic peptide
NIKH

SEQ ID NO: 269 - protein, 3 aa, Artificial Sequence
  Synthetic peptide
IKH

SEQ ID NO: 270 - protein, 10 aa, Artificial Sequence
  Synthetic peptide
CGSKDNIKHV

SEQ ID NO: 271 - protein, 8 aa, Artificial Sequence
  Synthetic peptide
SKDNIKHV

SEQ ID NO: 272 - protein, 7 aa, Artificial Sequence
  Synthetic peptide
KDNIKHV

SEQ ID NO: 273 - protein, 6 aa, Artificial Sequence
  Synthetic peptide
DNIKHV

SEQ ID NO: 274 - protein, 5 aa, Artificial Sequence
  Synthetic peptide
NIKHV

SEQ ID NO: 275 - protein, 4 aa, Artificial Sequence
  Synthetic peptide
IKHV

SEQ ID NO: 276 - protein, 3 aa, Artificial Sequence
  Synthetic peptide
KHV

SEQ ID NO: 277 - protein, 9 aa, Artificial Sequence
  Synthetic peptide
SKDNIKHVP

SEQ ID NO: 278 - protein, 8 aa, Artificial Sequence
  Synthetic peptide
KDNIKHVP

SEQ ID NO: 279 - protein, 7 aa, Artificial Sequence
  Synthetic peptide
DNIKHVP

SEQ ID NO: 280 - protein, 5 aa, Artificial Sequence
  Synthetic peptide
IKHVP

SEQ ID NO: 281 - protein, 4 aa, Artificial Sequence
  Synthetic peptide
KHVP

SEQ ID NO: 282 - protein, 3 aa, Artificial Sequence
  Synthetic peptide
HVP

SEQ ID NO: 283 - protein, 10 aa, Artificial Sequence
  Synthetic peptide
SKDNIKHVPG

SEQ ID NO: 284 - protein, 9 aa, Artificial Sequence
  Synthetic peptide
KDNIKHVPG

SEQ ID NO: 285 - protein, 8 aa, Artificial Sequence
  Synthetic peptide
DNIKHVPG

SEQ ID NO: 286 - protein, 6 aa, Artificial Sequence
  Synthetic peptide
IKHVPG

SEQ ID NO: 287 - protein, 5 aa, Artificial Sequence
  Synthetic peptide
KHVPG

SEQ ID NO: 288 - protein, 4 aa, Artificial Sequence
  Synthetic peptide
HVPG

SEQ ID NO: 289 - protein, 3 aa, Artificial Sequence
  Synthetic peptide
VPG

SEQ ID NO: 290 - protein, 10 aa, Artificial Sequence
  Synthetic peptide
KDNIKHVPGG

SEQ ID NO: 291 - protein, 9 aa, Artificial Sequence
  Synthetic peptide
DNIKHVPGG

SEQ ID NO: 292 - protein, 8 aa, Artificial Sequence
  Synthetic peptide
NIKHVPGG

SEQ ID NO: 293 - protein, 7 aa, Artificial Sequence
  Synthetic peptide
IKHVPGG

SEQ ID NO: 294 - protein, 3 aa, Artificial Sequence
  Synthetic peptide
PGG

SEQ ID NO: 295 - protein, 10 aa, Artificial Sequence
  Synthetic peptide
DNIKHVPGGG

SEQ ID NO: 296 - protein, 9 aa, Artificial Sequence
  Synthetic peptide
NIKHVPGGG

SEQ ID NO: 297 - protein, 8 aa, Artificial Sequence
  Synthetic peptide
IKHVPGGG

SEQ ID NO: 298 - protein, 5 aa, Artificial Sequence
  Synthetic peptide
VPGGG

SEQ ID NO: 299 - protein, 4 aa, Artificial Sequence
  Synthetic peptide
PGGG

SEQ ID NO: 300 - protein, 10 aa, Artificial Sequence
  Synthetic peptide
NIKHVPGGGS

SEQ ID NO: 301 - protein, 9 aa, Artificial Sequence
  Synthetic peptide
IKHVPGGGS

SEQ ID NO: 302 - protein, 8 aa, Artificial Sequence
  Synthetic peptide
KHVPGGGS

SEQ ID NO: 303 - protein, 7 aa, Artificial Sequence
  Synthetic peptide
HVPGGGS

SEQ ID NO: 304 - protein, 6 aa, Artificial Sequence
  Synthetic peptide
VPGGGS

SEQ ID NO: 305 - protein, 5 aa, Artificial Sequence
  Synthetic peptide
PGGGS

SEQ ID NO: 306 - protein, 4 aa, Artificial Sequence
  Synthetic peptide
GGGS

SEQ ID NO: 307 - protein, 10 aa, Artificial Sequence
  Synthetic peptide
IKHVPGGGSV

SEQ ID NO: 308 - protein, 9 aa, Artificial Sequence
  Synthetic peptide
KHVPGGGSV

SEQ ID NO: 309 - protein, 8 aa, Artificial Sequence
  Synthetic peptide
HVPGGGSV

SEQ ID NO: 310 - protein, 7 aa, Artificial Sequence
  Synthetic peptide
VPGGGSV

SEQ ID NO: 311 - protein, 6 aa, Artificial Sequence
  Synthetic peptide
PGGGSV

SEQ ID NO: 312 - protein, 5 aa, Artificial Sequence
  Synthetic peptide
GGGSV

SEQ ID NO: 313 - protein, 10 aa, Artificial Sequence
  Synthetic peptide
KHVPGGGSVQ

SEQ ID NO: 314 - protein, 9 aa, Artificial Sequence
  Synthetic peptide
HVPGGGSVQ

SEQ ID NO: 315 - protein, 8 aa, Artificial Sequence
  Synthetic peptide
VPGGGSVQ

SEQ ID NO: 316 - protein, 7 aa, Artificial Sequence
  Synthetic peptide
PGGGSVQ

SEQ ID NO: 317 - protein, 6 aa, Artificial Sequence
  Synthetic peptide
GGGSVQ

SEQ ID NO: 318 - protein, 10 aa, Artificial Sequence
  Synthetic peptide
HVPGGGSVQI

SEQ ID NO: 319 - protein, 9 aa, Artificial Sequence
  Synthetic peptide
VPGGGSVQI

SEQ ID NO: 320 - protein, 8 aa, Artificial Sequence
  Synthetic peptide
PGGGSVQI

SEQ ID NO: 321 - protein, 10 aa, Artificial Sequence
  Synthetic peptide
GGSVQIVYKS

SEQ ID NO: 322 - protein, 9 aa, Artificial Sequence
  Synthetic peptide
GSVQIVYKS

SEQ ID NO: 323 - protein, 8 aa, Artificial Sequence
  Synthetic peptide
SVQIVYKS

SEQ ID NO: 324 - protein, 7 aa, Artificial Sequence
  Synthetic peptide
VQIVYKS

SEQ ID NO: 325 - protein, 6 aa, Artificial Sequence
  Synthetic peptide
QIVYKS

SEQ ID NO: 326 - protein, 10 aa, Artificial Sequence
  Synthetic peptide
GSVQIVYKSV

SEQ ID NO: 327 - protein, 9 aa, Artificial Sequence
  Synthetic peptide
SVQIVYKSV

SEQ ID NO: 328 - protein, 8 aa, Artificial Sequence
  Synthetic peptide
VQIVYKSV

SEQ ID NO: 329 - protein, 6 aa, Artificial Sequence
  Synthetic peptide
IVYKSV

SEQ ID NO: 330 - protein, 5 aa, Artificial Sequence
  Synthetic peptide
VYKSV

SEQ ID NO: 331 - protein, 4 aa, Artificial Sequence
  Synthetic peptide
YKSV

SEQ ID NO: 332 - protein, 3 aa, Artificial Sequence
  Synthetic peptide
KSV

SEQ ID NO: 333 - protein, 10 aa, Artificial Sequence
  Synthetic peptide
SVQIVYKSVD

SEQ ID NO: 334 - protein, 9 aa, Artificial Sequence
  Synthetic peptide
VQIVYKSVD

SEQ ID NO: 335 - protein, 8 aa, Artificial Sequence
  Synthetic peptide
QIVYKSVD

SEQ ID NO: 336 - protein, 7 aa, Artificial Sequence
  Synthetic peptide
IVYKSVD

SEQ ID NO: 337 - protein, 6 aa, Artificial Sequence
  Synthetic peptide
VYKSVD

SEQ ID NO: 338 - protein, 5 aa, Artificial Sequence
  Synthetic peptide
YKSVD

SEQ ID NO: 339 - protein, 4 aa, Artificial Sequence
  Synthetic peptide
KSVD

SEQ ID NO: 340 - protein, 3 aa, Artificial Sequence
  Synthetic peptide
SVD

SEQ ID NO: 341 - protein, 10 aa, Artificial Sequence
  Synthetic peptide
VQIVYKSVDL

SEQ ID NO: 342 - protein, 9 aa, Artificial Sequence
  Synthetic peptide
QIVYKSVDL

SEQ ID NO: 343 - protein, 8 aa, Artificial Sequence
  Synthetic peptide
IVYKSVDL

SEQ ID NO: 344 - protein, 7 aa, Artificial Sequence
  Synthetic peptide
VYKSVDL

SEQ ID NO: 345 - protein, 6 aa, Artificial Sequence
  Synthetic peptide
YKSVDL

SEQ ID NO: 346 - protein, 5 aa, Artificial Sequence
  Synthetic peptide
KSVDL

SEQ ID NO: 347 - protein, 4 aa, Artificial Sequence
  Synthetic peptide
SVDL

SEQ ID NO: 348 - protein, 10 aa, Artificial Sequence
  Synthetic peptide
QIVYKSVDLS

SEQ ID NO: 349 - protein, 9 aa, Artificial Sequence
  Synthetic peptide
IVYKSVDLS

SEQ ID NO: 350 - protein, 8 aa, Artificial Sequence
  Synthetic peptide
VYKSVDLS

SEQ ID NO: 351 - protein, 7 aa, Artificial Sequence
  Synthetic peptide
YKSVDLS

SEQ ID NO: 352 - protein, 6 aa, Artificial Sequence
  Synthetic peptide
KSVDLS

SEQ ID NO: 353 - protein, 5 aa, Artificial Sequence
  Synthetic peptide
SVDLS

SEQ ID NO: 354 - protein, 10 aa, Artificial Sequence
  Synthetic peptide
IVYKSVDLSK

SEQ ID NO: 355 - protein, 9 aa, Artificial Sequence
  Synthetic peptide
VYKSVDLSK

SEQ ID NO: 356 - protein, 8 aa, Artificial Sequence
  Synthetic peptide
YKSVDLSK

SEQ ID NO: 357 - protein, 7 aa, Artificial Sequence
  Synthetic peptide
KSVDLSK

SEQ ID NO: 358 - protein, 6 aa, Artificial Sequence
  Synthetic peptide
SVDLSK

SEQ ID NO: 359 - protein, 10 aa, Artificial Sequence
  Synthetic peptide
VYKSVDLSKV

SEQ ID NO: 360 - protein, 9 aa, Artificial Sequence
  Synthetic peptide
YKSVDLSKV

SEQ ID NO: 361 - protein, 8 aa, Artificial Sequence
  Synthetic peptide
KSVDLSKV

SEQ ID NO: 362 - protein, 7 aa, Artificial Sequence
  Synthetic peptide
SVDLSKV

SEQ ID NO: 363 - protein, 10 aa, Artificial Sequence
  Synthetic peptide
YKSVDLSKVT

SEQ ID NO: 364 - protein, 9 aa, Artificial Sequence
  Synthetic peptide
KSVDLSKVT

SEQ ID NO: 365 - protein, 8 aa, Artificial Sequence
  Synthetic peptide
SVDLSKVT

SEQ ID NO: 366 - protein, 10 aa, Artificial Sequence
  Synthetic peptide
HGAEIVYKSV

SEQ ID NO: 367 - protein, 9 aa, Artificial Sequence
  Synthetic peptide
GAEIVYKSV

SEQ ID NO: 368 - protein, 8 aa, Artificial Sequence
  Synthetic peptide
AEIVYKSV

SEQ ID NO: 369 - protein, 10 aa, Artificial Sequence
  Synthetic peptide
GAEIVYKSVV

SEQ ID NO: 370 - protein, 9 aa, Artificial Sequence
  Synthetic peptide
AEIVYKSVV

SEQ ID NO: 371 - protein, 8 aa, Artificial Sequence
  Synthetic peptide
EIVYKSVV

SEQ ID NO: 372 - protein, 7 aa, Artificial Sequence
  Synthetic peptide
IVYKSVV

SEQ ID NO: 373 - protein, 6 aa, Artificial Sequence
  Synthetic peptide
VYKSVV

SEQ ID NO: 374 - protein, 5 aa, Artificial Sequence
  Synthetic peptide
YKSVV

SEQ ID NO: 375 - protein, 4 aa, Artificial Sequence
  Synthetic peptide
KSVV

SEQ ID NO: 376 - protein, 3 aa, Artificial Sequence
  Synthetic peptide
SVV

SEQ ID NO: 377 - protein, 10 aa, Artificial Sequence
  Synthetic peptide
AEIVYKSVVS

SEQ ID NO: 378 - protein, 9 aa, Artificial Sequence
  Synthetic peptide
EIVYKSVVS

SEQ ID NO: 379 - protein, 8 aa, Artificial Sequence
  Synthetic peptide
IVYKSVVS

SEQ ID NO: 380 - protein, 7 aa, Artificial Sequence
  Synthetic peptide
VYKSVVS

SEQ ID NO: 381 - protein, 6 aa, Artificial Sequence
  Synthetic peptide
YKSVVS

SEQ ID NO: 382 - protein, 5 aa, Artificial Sequence
  Synthetic peptide
KSVVS

SEQ ID NO: 383 - protein, 4 aa, Artificial Sequence
  Synthetic peptide
SVVS

SEQ ID NO: 384 - protein, 10 aa, Artificial Sequence
  Synthetic peptide
EIVYKSVVSG

SEQ ID NO: 385 - protein, 9 aa, Artificial Sequence
  Synthetic peptide
IVYKSVVSG

SEQ ID NO: 386 - protein, 8 aa, Artificial Sequence
  Synthetic peptide
VYKSVVSG

SEQ ID NO: 387 - protein, 7 aa, Artificial Sequence
  Synthetic peptide
YKSVVSG

SEQ ID NO: 388 - protein, 6 aa, Artificial Sequence
  Synthetic peptide
KSVVSG

SEQ ID NO: 389 - protein, 5 aa, Artificial Sequence
  Synthetic peptide
SVVSG

SEQ ID NO: 390 - protein, 4 aa, Artificial Sequence
  Synthetic peptide
VVSG

SEQ ID NO: 391 - protein, 10 aa, Artificial Sequence
  Synthetic peptide
IVYKSVVSGD

SEQ ID NO: 392 - protein, 9 aa, Artificial Sequence
  Synthetic peptide
VYKSVVSGD

SEQ ID NO: 393 - protein, 8 aa, Artificial Sequence
  Synthetic peptide
YKSVVSGD

SEQ ID NO: 394 - protein, 7 aa, Artificial Sequence
  Synthetic peptide
KSVVSGD

SEQ ID NO: 395 - protein, 6 aa, Artificial Sequence
  Synthetic peptide
SVVSGD

SEQ ID NO: 396 - protein, 10 aa, Artificial Sequence
  Synthetic peptide
VYKSVVSGDT

SEQ ID NO: 397 - protein, 9 aa, Artificial Sequence
  Synthetic peptide
YKSVVSGDT

SEQ ID NO: 398 - protein, 8 aa, Artificial Sequence
  Synthetic peptide
KSVVSGDT

SEQ ID NO: 399 - protein, 7 aa, Artificial Sequence
  Synthetic peptide
SVVSGDT

SEQ ID NO: 400 - protein, 6 aa, Artificial Sequence
  Synthetic peptide
VVSGDT

SEQ ID NO: 401 - protein, 10 aa, Artificial Sequence
  Synthetic peptide
YKSVVSGDTS

SEQ ID NO: 402 - protein, 9 aa, Artificial Sequence
  Synthetic peptide
KSVVSGDTS

SEQ ID NO: 403 - protein, 8 aa, Artificial Sequence
  Synthetic peptide
SVVSGDTS

SEQ ID NO: 404 - protein, 10 aa, Artificial Sequence
  Synthetic peptide
KSVVSGDTSP

SEQ ID NO: 405 - protein, 10 aa, Artificial Sequence
  Synthetic peptide
SVVSGDTSPR

SEQ ID NO: 406 - protein, 9 aa, Artificial Sequence
  Synthetic peptide
VVSGDTSPR

SEQ ID NO: 407 - protein, 10 aa, Artificial Sequence
  Synthetic peptide
DHGAEIVYKP

SEQ ID NO: 408 - protein, 9 aa, Artificial Sequence
  Synthetic peptide
HGAEIVYKP

SEQ ID NO: 409 - protein, 8 aa, Artificial Sequence
  Synthetic peptide
GAEIVYKP

SEQ ID NO: 410 - protein, 7 aa, Artificial Sequence
  Synthetic peptide
AEIVYKP

SEQ ID NO: 411 - protein, 6 aa, Artificial Sequence
  Synthetic peptide
EIVYKP

SEQ ID NO: 412 - protein, 10 aa, Artificial Sequence
  Synthetic peptide
HGAEIVYKPV

SEQ ID NO: 413 - protein, 9 aa, Artificial Sequence
  Synthetic peptide
GAEIVYKPV

SEQ ID NO: 414 - protein, 8 aa, Artificial Sequence
  Synthetic peptide
AEIVYKPV

SEQ ID NO: 415 - protein, 10 aa, Artificial Sequence
  Synthetic peptide
GAEIVYKPVV

SEQ ID NO: 416 - protein, 9 aa, Artificial Sequence
  Synthetic peptide
AEIVYKPVV

SEQ ID NO: 417 - protein, 8 aa, Artificial Sequence
  Synthetic peptide
EIVYKPVV

SEQ ID NO: 418 - protein, 7 aa, Artificial Sequence
  Synthetic peptide
IVYKPVV

SEQ ID NO: 419 - protein, 6 aa, Artificial Sequence
  Synthetic peptide
VYKPVV

SEQ ID NO: 420 - protein, 5 aa, Artificial Sequence
  Synthetic peptide
YKPVV

SEQ ID NO: 421 - protein, 4 aa, Artificial Sequence
  Synthetic peptide
KPVV

SEQ ID NO: 422 - protein, 10 aa, Artificial Sequence
  Synthetic peptide
AEIVYKPVVS

SEQ ID NO: 423 - protein, 9 aa, Artificial Sequence
  Synthetic peptide
EIVYKPVVS

SEQ ID NO: 424 - protein, 8 aa, Artificial Sequence
  Synthetic peptide
IVYKPVVS

SEQ ID NO: 425 - protein, 7 aa, Artificial Sequence
  Synthetic peptide
VYKPVVS

SEQ ID NO: 426 - protein, 6 aa, Artificial Sequence
  Synthetic peptide
YKPVVS

SEQ ID NO: 427 - protein, 5 aa, Artificial Sequence
  Synthetic peptide
KPVVS

SEQ ID NO: 428 - protein, 4 aa, Artificial Sequence
  Synthetic peptide
PVVS

SEQ ID NO: 429 - protein, 10 aa, Artificial Sequence
  Synthetic peptide
EIVYKPVVSG

SEQ ID NO: 430 - protein, 9 aa, Artificial Sequence
  Synthetic peptide
IVYKPVVSG

SEQ ID NO: 431 - protein, 8 aa, Artificial Sequence
  Synthetic peptide
VYKPVVSG

SEQ ID NO: 432 - protein, 7 aa, Artificial Sequence
  Synthetic peptide
YKPVVSG

SEQ ID NO: 433 - protein, 6 aa, Artificial Sequence
  Synthetic peptide
KPVVSG

SEQ ID NO: 434 - protein, 4 aa, Artificial Sequence
  Synthetic peptide
VVSG

SEQ ID NO: 435 - protein, 10 aa, Artificial Sequence
  Synthetic peptide
IVYKPVVSGD

SEQ ID NO: 436 - protein, 9 aa, Artificial Sequence
  Synthetic peptide
VYKPVVSGD

SEQ ID NO: 437 - protein, 8 aa, Artificial Sequence
  Synthetic peptide
YKPVVSGD

SEQ ID NO: 438 - protein, 7 aa, Artificial Sequence
  Synthetic peptide
KPVVSGD

SEQ ID NO: 439 - protein, 10 aa, Artificial Sequence
  Synthetic peptide
VYKPVVSGDT

SEQ ID NO: 440 - protein, 9 aa, Artificial Sequence
  Synthetic peptide
YKPVVSGDT

SEQ ID NO: 441 - protein, 8 aa, Artificial Sequence
  Synthetic peptide
KPVVSGDT

SEQ ID NO: 442 - protein, 10 aa, Artificial Sequence
  Synthetic peptide
YKPVVSGDTS

SEQ ID NO: 443 - protein, 10 aa, Artificial Sequence
  Synthetic peptide
KPVVSGDTSP

SEQ ID NO: 444 - protein, 4 aa, Artificial Sequence
  Synthetic peptide
CNIK

SEQ ID NO: 445 - protein, 5 aa, Artificial Sequence
  Synthetic peptide
CNIKH

SEQ ID NO: 446 - protein, 6 aa, Artificial Sequence
  Synthetic peptide
CNIKHV

SEQ ID NO: 447 - protein, 9 aa, Artificial Sequence
  Synthetic peptide
CNIKHVPGG

SEQ ID NO: 448 - protein, 10 aa, Artificial Sequence
  Synthetic peptide
CNIKHVPGGG

SEQ ID NO: 449 - protein, 9 aa, Artificial Sequence
  Synthetic peptide
EAAGHVTQC

SEQ ID NO: 450 - protein, 10 aa, Artificial Sequence
  Synthetic peptide
EAAGHVTQAR

SEQ ID NO: 451 - protein, 9 aa, Artificial Sequence
  Synthetic peptide
AAGHVTQAC

SEQ ID NO: 452 - protein, 9 aa, Artificial Sequence
  Synthetic peptide
AGHVTQARC

SEQ ID NO: 453 - protein, 8 aa, Artificial Sequence
  Synthetic peptide
AGHVTQAR

SEQ ID NO: 454 - protein, 7 aa, Artificial Sequence
  Synthetic peptide
GYTMHQD

SEQ ID NO: 455 - protein, 8 aa, Artificial Sequence
  Synthetic peptide
QGGYTMHC

SEQ ID NO: 456 - protein, 9 aa, Artificial Sequence
  Synthetic peptide
QGGYTMHQD

SEQ ID NO: 457 - protein, 8 aa, Artificial Sequence
  Synthetic peptide
GGYTMHQC

SEQ ID NO: 458 - protein, 10 aa, Artificial Sequence
  Synthetic peptide
ENLKHQPGGG

SEQ ID NO: 459 - protein, 9 aa, Artificial Sequence
  Synthetic peptide
NLKHQPGGG

SEQ ID NO: 460 - protein, 8 aa, Artificial Sequence
  Synthetic peptide
LKHQPGGG

SEQ ID NO: 461 - protein, 7 aa, Artificial Sequence
  Synthetic peptide
KHQPGGG

SEQ ID NO: 462 - protein, 5 aa, Artificial Sequence
  Synthetic peptide
QPGGG

SEQ ID NO: 463 - protein, 10 aa, Artificial Sequence
  Synthetic peptide
TENLKHQPGG

SEQ ID NO: 464 - protein, 9 aa, Artificial Sequence
  Synthetic peptide
ENLKHQPGG

SEQ ID NO: 465 - protein, 8 aa, Artificial Sequence
  Synthetic peptide
NLKHQPGG

SEQ ID NO: 466 - protein, 7 aa, Artificial Sequence
  Synthetic peptide
LKHQPGG

SEQ ID NO: 467 - protein, 6 aa, Artificial Sequence
  Synthetic peptide
KHQPGG

SEQ ID NO: 468 - protein, 4 aa, Artificial Sequence
  Synthetic peptide
QPGG

SEQ ID NO: 469 - protein, 9 aa, Artificial Sequence
  Synthetic peptide
TENLKHQPG

SEQ ID NO: 470 - protein, 8 aa, Artificial Sequence
  Synthetic peptide
ENLKHQPG

SEQ ID NO: 471 - protein, 7 aa, Artificial Sequence
  Synthetic peptide
NLKHQPG

SEQ ID NO: 472 - protein, 6 aa, Artificial Sequence
  Synthetic peptide
LKHQPG

SEQ ID NO: 473 - protein, 5 aa, Artificial Sequence
  Synthetic peptide
KHQPG

SEQ ID NO: 474 - protein, 4 aa, Artificial Sequence
  Synthetic peptide
HQPG

SEQ ID NO: 475 - protein, 3 aa, Artificial Sequence
  Synthetic peptide
QPG

SEQ ID NO: 476 - protein, 8 aa, Artificial Sequence
  Synthetic peptide
TENLKHQP

SEQ ID NO: 477 - protein, 7 aa, Artificial Sequence
  Synthetic peptide
ENLKHQP

SEQ ID NO: 478 - protein, 6 aa, Artificial Sequence
  Synthetic peptide
NLKHQP

SEQ ID NO: 479 - protein, 5 aa, Artificial Sequence
  Synthetic peptide
LKHQP

SEQ ID NO: 480 - protein, 4 aa, Artificial Sequence
  Synthetic peptide
KHQP

SEQ ID NO: 481 - protein, 3 aa, Artificial Sequence
  Synthetic peptide
HQP

SEQ ID NO: 482 - protein, 7 aa, Artificial Sequence
  Synthetic peptide
TENLKHQ

SEQ ID NO: 483 - protein, 6 aa, Artificial Sequence
  Synthetic peptide
ENLKHQ

SEQ ID NO: 484 - protein, 5 aa, Artificial Sequence
  Synthetic peptide
NLKHQ

SEQ ID NO: 485 - protein, 4 aa, Artificial Sequence
  Synthetic peptide
LKHQ

SEQ ID NO: 486 - protein, 3 aa, Artificial Sequence
  Synthetic peptide
KHQ

SEQ ID NO: 487 - protein, 6 aa, Artificial Sequence
  Synthetic peptide
TENLKH

SEQ ID NO: 488 - protein, 5 aa, Artificial Sequence
  Synthetic peptide
ENLKH

SEQ ID NO: 489 - protein, 4 aa, Artificial Sequence
  Synthetic peptide
NLKH

SEQ ID NO: 490 - protein, 3 aa, Artificial Sequence
  Synthetic peptide
LKH

SEQ ID NO: 491 - protein, 5 aa, Artificial Sequence
  Synthetic peptide
TENLK

SEQ ID NO: 492 - protein, 4 aa, Artificial Sequence
  Synthetic peptide
ENLK

SEQ ID NO: 493 - protein, 3 aa, Artificial Sequence
  Synthetic peptide
NLK

SEQ ID NO: 494 - protein, 4 aa, Artificial Sequence
  Synthetic peptide
TENL

SEQ ID NO: 495 - protein, 3 aa, Artificial Sequence
  Synthetic peptide
ENL

SEQ ID NO: 496 - protein, 3 aa, Artificial Sequence
  Synthetic peptide
TEN

SEQ ID NO: 497 - protein, 4 aa, Artificial Sequence
  Synthetic peptide
KDNI

SEQ ID NO: 498 - protein, 3 aa, Artificial Sequence
  Synthetic peptide
DNI

SEQ ID NO: 499 - protein, 3 aa, Artificial Sequence
  Synthetic peptide
KDN

SEQ ID NO: 500 - protein, 7 aa, Artificial Sequence
  Synthetic peptide
IKHVGGG

SEQ ID NO: 501 - protein, 6 aa, Artificial Sequence
  Synthetic peptide
IKHVGG

SEQ ID NO: 502 - protein, 5 aa, Artificial Sequence
  Synthetic peptide
IKHVG

SEQ ID NO: 503 - protein, 6 aa, Artificial Sequence
  Synthetic peptide
KHVGGG

SEQ ID NO: 504 - protein, 5 aa, Artificial Sequence
  Synthetic peptide
KHVGG

SEQ ID NO: 505 - protein, 4 aa, Artificial Sequence
  Synthetic peptide
KHVG

SEQ ID NO: 506 - protein, 10 aa, Artificial Sequence
  Synthetic peptide
GNIHHKPGGG

SEQ ID NO: 507 - protein, 9 aa, Artificial Sequence
  Synthetic peptide
NIHHKPGGG

SEQ ID NO: 508 - protein, 8 aa, Artificial Sequence
  Synthetic peptide
IHHKPGGG

SEQ ID NO: 509 - protein, 7 aa, Artificial Sequence
  Synthetic peptide
HHKPGGG

SEQ ID NO: 510 - protein, 5 aa, Artificial Sequence
  Synthetic peptide
KPGGG

SEQ ID NO: 511 - protein, 10 aa, Artificial Sequence
  Synthetic peptide
LGNIHHKPGG

SEQ ID NO: 512 - protein, 9 aa, Artificial Sequence
  Synthetic peptide
GNIHHKPGG

SEQ ID NO: 513 - protein, 8 aa, Artificial Sequence
  Synthetic peptide
NIHHKPGG

SEQ ID NO: 514 - protein, 7 aa, Artificial Sequence
  Synthetic peptide
IHHKPGG

SEQ ID NO: 515 - protein, 6 aa, Artificial Sequence
  Synthetic peptide
HHKPGG

SEQ ID NO: 516 - protein, 4 aa, Artificial Sequence
  Synthetic peptide
KPGG

SEQ ID NO: 517 - protein, 9 aa, Artificial Sequence
  Synthetic peptide
LGNIHHKPG

SEQ ID NO: 518 - protein, 8 aa, Artificial Sequence
  Synthetic peptide
GNIHHKPG

SEQ ID NO: 519 - protein, 7 aa, Artificial Sequence
  Synthetic peptide
NIHHKPG

SEQ ID NO: 520 - protein, 6 aa, Artificial Sequence
  Synthetic peptide
IHHKPG

SEQ ID NO: 521 - protein, 5 aa, Artificial Sequence
  Synthetic peptide
HHKPG

SEQ ID NO: 522 - protein, 4 aa, Artificial Sequence
  Synthetic peptide
HKPG

SEQ ID NO: 523 - protein, 3 aa, Artificial Sequence
  Synthetic peptide
KPG

SEQ ID NO: 524 - protein, 8 aa, Artificial Sequence
  Synthetic peptide
LGNIHHKP

SEQ ID NO: 525 - protein, 7 aa, Artificial Sequence
  Synthetic peptide
GNIHHKP

SEQ ID NO: 526 - protein, 6 aa, Artificial Sequence
  Synthetic peptide
NIHHKP

SEQ ID NO: 527 - protein, 5 aa, Artificial Sequence
  Synthetic peptide
IHHKP

SEQ ID NO: 528 - protein, 4 aa, Artificial Sequence
  Synthetic peptide
HHKP

SEQ ID NO: 529 - protein, 3 aa, Artificial Sequence
  Synthetic peptide
HKP

SEQ ID NO: 530 - protein, 7 aa, Artificial Sequence
  Synthetic peptide
LGNIHHK

SEQ ID NO: 531 - protein, 6 aa, Artificial Sequence
  Synthetic peptide
GNIHHK

SEQ ID NO: 532 - protein, 5 aa, Artificial Sequence
  Synthetic peptide
NIHHK

SEQ ID NO: 533 - protein, 4 aa, Artificial Sequence
  Synthetic peptide
IHHK

SEQ ID NO: 534 - protein, 3 aa, Artificial Sequence
  Synthetic peptide
HHK

SEQ ID NO: 535 - protein, 6 aa, Artificial Sequence
  Synthetic peptide
LGNIHH

SEQ ID NO: 536 - protein, 5 aa, Artificial Sequence
  Synthetic peptide
GNIHH

SEQ ID NO: 537 - protein, 4 aa, Artificial Sequence
  Synthetic peptide
NIHH

SEQ ID NO: 538 - protein, 3 aa, Artificial Sequence
  Synthetic peptide
IHH

SEQ ID NO: 539 - protein, 5 aa, Artificial Sequence
  Synthetic peptide
LGNIH

SEQ ID NO: 540 - protein, 4 aa, Artificial Sequence
  Synthetic peptide
GNIH

SEQ ID NO: 541 - protein, 3 aa, Artificial Sequence
  Synthetic peptide
NIH

SEQ ID NO: 542 - protein, 4 aa, Artificial Sequence
  Synthetic peptide
LGNI

SEQ ID NO: 543 - protein, 3 aa, Artificial Sequence
  Synthetic peptide
GNI

SEQ ID NO: 544 - protein, 3 aa, Artificial Sequence
  Synthetic peptide
LGN

SEQ ID NO: 545 - protein, 10 aa, Artificial Sequence
  Synthetic peptide
DNITHVPGGG

SEQ ID NO: 546 - protein, 9 aa, Artificial Sequence
  Synthetic peptide
NITHVPGGG

SEQ ID NO: 547 - protein, 8 aa, Artificial Sequence
  Synthetic peptide
ITHVPGGG

SEQ ID NO: 548 - protein, 7 aa, Artificial Sequence
  Synthetic peptide
THVPGGG

SEQ ID NO: 549 - protein, 10 aa, Artificial Sequence
  Synthetic peptide
LDNITHVPGG

SEQ ID NO: 550 - protein, 9 aa, Artificial Sequence
  Synthetic peptide
DNITHVPGG

SEQ ID NO: 551 - protein, 8 aa, Artificial Sequence
  Synthetic peptide
NITHVPGG

SEQ ID NO: 552 - protein, 7 aa, Artificial Sequence
  Synthetic peptide
ITHVPGG

SEQ ID NO: 553 - protein, 6 aa, Artificial Sequence
  Synthetic peptide
THVPGG

SEQ ID NO: 554 - protein, 9 aa, Artificial Sequence
  Synthetic peptide
LDNITHVPG

SEQ ID NO: 555 - protein, 8 aa, Artificial Sequence
  Synthetic peptide
DNITHVPG

SEQ ID NO: 556 - protein, 7 aa, Artificial Sequence
  Synthetic peptide
NITHVPG

SEQ ID NO: 557 - protein, 6 aa, Artificial Sequence
  Synthetic peptide
ITHVPG

SEQ ID NO: 558 - protein, 5 aa, Artificial Sequence
  Synthetic peptide
THVPG

SEQ ID NO: 559 - protein, 4 aa, Artificial Sequence
  Synthetic peptide
HVPG

SEQ ID NO: 560 - protein, 3 aa, Artificial Sequence
  Synthetic peptide
VPG

SEQ ID NO: 561 - protein, 8 aa, Artificial Sequence
  Synthetic peptide
LDNITHVP

SEQ ID NO: 562 - protein, 7 aa, Artificial Sequence
  Synthetic peptide
DNITHVP

SEQ ID NO: 563 - protein, 6 aa, Artificial Sequence
  Synthetic peptide
NITHVP

SEQ ID NO: 564 - protein, 5 aa, Artificial Sequence
  Synthetic peptide
ITHVP

SEQ ID NO: 565 - protein, 4 aa, Artificial Sequence
  Synthetic peptide
THVP

SEQ ID NO: 566 - protein, 7 aa, Artificial Sequence
  Synthetic peptide
LDNITHV

SEQ ID NO: 567 - protein, 6 aa, Artificial Sequence
  Synthetic peptide
DNITHV

SEQ ID NO: 568 - protein, 5 aa, Artificial Sequence
  Synthetic peptide
NITHV

SEQ ID NO: 569 - protein, 4 aa, Artificial Sequence
  Synthetic peptide
ITHV

SEQ ID NO: 570 - protein, 3 aa, Artificial Sequence
  Synthetic peptide
THV

SEQ ID NO: 571 - protein, 6 aa, Artificial Sequence
  Synthetic peptide
LDNITH

SEQ ID NO: 572 - protein, 5 aa, Artificial Sequence
  Synthetic peptide
DNITH

SEQ ID NO: 573 - protein, 4 aa, Artificial Sequence
  Synthetic peptide
NITH

SEQ ID NO: 574 - protein, 3 aa, Artificial Sequence
  Synthetic peptide
ITH

SEQ ID NO: 575 - protein, 5 aa, Artificial Sequence
  Synthetic peptide
LDNIT

SEQ ID NO: 576 - protein, 4 aa, Artificial Sequence
  Synthetic peptide
DNIT

SEQ ID NO: 577 - protein, 3 aa, Artificial Sequence
  Synthetic peptide
NIT

SEQ ID NO: 578 - protein, 4 aa, Artificial Sequence
  Synthetic peptide
LDNI

SEQ ID NO: 579 - protein, 3 aa, Artificial Sequence
  Synthetic peptide
LDN

SEQ ID NO: 580 - protein, 10 aa, Artificial Sequence
  Synthetic peptide
KNVKSKIGST

SEQ ID NO: 581 - protein, 9 aa, Artificial Sequence
  Synthetic peptide
NVKSKIGST

SEQ ID NO: 582 - protein, 8 aa, Artificial Sequence
  Synthetic peptide
VKSKIGST

SEQ ID NO: 583 - protein, 7 aa, Artificial Sequence
  Synthetic peptide
KSKIGST

SEQ ID NO: 584 - protein, 6 aa, Artificial Sequence
  Synthetic peptide
SKIGST

SEQ ID NO: 585 - protein, 5 aa, Artificial Sequence
  Synthetic peptide
KIGST

SEQ ID NO: 586 - protein, 4 aa, Artificial Sequence
  Synthetic peptide
IGST

SEQ ID NO: 587 - protein, 3 aa, Artificial Sequence
  Synthetic peptide
GST

SEQ ID NO: 588 - protein, 10 aa, Artificial Sequence
  Synthetic peptide
NVKSKIGSTE

SEQ ID NO: 589 - protein, 9 aa, Artificial Sequence
  Synthetic peptide
VKSKIGSTE

SEQ ID NO: 590 - protein, 8 aa, Artificial Sequence
  Synthetic peptide
KSKIGSTE

SEQ ID NO: 591 - protein, 7 aa, Artificial Sequence
  Synthetic peptide
SKIGSTE

SEQ ID NO: 592 - protein, 6 aa, Artificial Sequence
  Synthetic peptide
KIGSTE

SEQ ID NO: 593 - protein, 5 aa, Artificial Sequence
  Synthetic peptide
IGSTE

SEQ ID NO: 594 - protein, 4 aa, Artificial Sequence
  Synthetic peptide
GSTE

SEQ ID NO: 595 - protein, 3 aa, Artificial Sequence
  Synthetic peptide
STE

SEQ ID NO: 596 - protein, 10 aa, Artificial Sequence
  Synthetic peptide
VKSKIGSTEN

SEQ ID NO: 597 - protein, 9 aa, Artificial Sequence
  Synthetic peptide
KSKIGSTEN

SEQ ID NO: 598 - protein, 8 aa, Artificial Sequence
  Synthetic peptide
SKIGSTEN

SEQ ID NO: 599 - protein, 7 aa, Artificial Sequence
  Synthetic peptide
KIGSTEN

SEQ ID NO: 600 - protein, 6 aa, Artificial Sequence
  Synthetic peptide
IGSTEN

SEQ ID NO: 601 - protein, 5 aa, Artificial Sequence
  Synthetic peptide
GSTEN

SEQ ID NO: 602 - protein, 4 aa, Artificial Sequence
  Synthetic peptide
STEN

SEQ ID NO: 603 - protein, 10 aa, Artificial Sequence
  Synthetic peptide
KSKIGSTENL

SEQ ID NO: 604 - protein, 9 aa, Artificial Sequence
  Synthetic peptide
SKIGSTENL

SEQ ID NO: 605 - protein, 8 aa, Artificial Sequence
  Synthetic peptide
KIGSTENL

SEQ ID NO: 606 - protein, 7 aa, Artificial Sequence
  Synthetic peptide
IGSTENL

SEQ ID NO: 607 - protein, 6 aa, Artificial Sequence
  Synthetic peptide
GSTENL

SEQ ID NO: 608 - protein, 5 aa, Artificial Sequence
  Synthetic peptide
STENL

SEQ ID NO: 609 - protein, 10 aa, Artificial Sequence
  Synthetic peptide
SKIGSTENLK

SEQ ID NO: 610 - protein, 9 aa, Artificial Sequence
  Synthetic peptide
KIGSTENLK

SEQ ID NO: 611 - protein, 8 aa, Artificial Sequence
  Synthetic peptide
IGSTENLK

SEQ ID NO: 612 - protein, 7 aa, Artificial Sequence
  Synthetic peptide
GSTENLK

SEQ ID NO: 613 - protein, 6 aa, Artificial Sequence
  Synthetic peptide
STENLK

SEQ ID NO: 614 - protein, 10 aa, Artificial Sequence
  Synthetic peptide
KIGSTENLKH

SEQ ID NO: 615 - protein, 9 aa, Artificial Sequence
  Synthetic peptide
IGSTENLKH

SEQ ID NO: 616 - protein, 8 aa, Artificial Sequence
  Synthetic peptide
GSTENLKH

SEQ ID NO: 617 - protein, 7 aa, Artificial Sequence
  Synthetic peptide
STENLKH

SEQ ID NO: 618 - protein, 10 aa, Artificial Sequence
  Synthetic peptide
IGSTENLKHQ

SEQ ID NO: 619 - protein, 9 aa, Artificial Sequence
  Synthetic peptide
GSTENLKHQ

SEQ ID NO: 620 - protein, 8 aa, Artificial Sequence
  Synthetic peptide
STENLKHQ

SEQ ID NO: 621 - protein, 10 aa, Artificial Sequence
  Synthetic peptide
GSTENLKHQP

SEQ ID NO: 622 - protein, 9 aa, Artificial Sequence
  Synthetic peptide
STENLKHQP

SEQ ID NO: 623 - protein, 10 aa, Artificial Sequence
  Synthetic peptide
STENLKHQPG

SEQ ID NO: 624 - protein, 10 aa, Artificial Sequence
  Synthetic peptide
SNVQSKCGSK

SEQ ID NO: 625 - protein, 9 aa, Artificial Sequence
  Synthetic peptide
NVQSKCGSK

SEQ ID NO: 626 - protein, 8 aa, Artificial Sequence
  Synthetic peptide
VQSKCGSK

SEQ ID NO: 627 - protein, 7 aa, Artificial Sequence
  Synthetic peptide
QSKCGSK

SEQ ID NO: 628 - protein, 6 aa, Artificial Sequence
  Synthetic peptide
SKCGSK

SEQ ID NO: 629 - protein, 5 aa, Artificial Sequence
  Synthetic peptide
KCGSK

SEQ ID NO: 630 - protein, 4 aa, Artificial Sequence
  Synthetic peptide
CGSK

SEQ ID NO: 631 - protein, 3 aa, Artificial Sequence
  Synthetic peptide
GSK

SEQ ID NO: 632 - protein, 10 aa, Artificial Sequence
  Synthetic peptide
NVQSKCGSKD

SEQ ID NO: 633 - protein, 9 aa, Artificial Sequence
  Synthetic peptide
VQSKCGSKD

SEQ ID NO: 634 - protein, 8 aa, Artificial Sequence
  Synthetic peptide
QSKCGSKD

SEQ ID NO: 635 - protein, 7 aa, Artificial Sequence
  Synthetic peptide
SKCGSKD

SEQ ID NO: 636 - protein, 6 aa, Artificial Sequence
  Synthetic peptide
KCGSKD

SEQ ID NO: 637 - protein, 5 aa, Artificial Sequence
  Synthetic peptide
CGSKD

SEQ ID NO: 638 - protein, 4 aa, Artificial Sequence
  Synthetic peptide
GSKD

SEQ ID NO: 639 - protein, 3 aa, Artificial Sequence
  Synthetic peptide
SKD

SEQ ID NO: 640 - protein, 10 aa, Artificial Sequence
  Synthetic peptide
VQSKCGSKDN

SEQ ID NO: 641 - protein, 9 aa, Artificial Sequence
  Synthetic peptide
QSKCGSKDN

SEQ ID NO: 642 - protein, 8 aa, Artificial Sequence
  Synthetic peptide
SKCGSKDN

SEQ ID NO: 643 - protein, 7 aa, Artificial Sequence
  Synthetic peptide
KCGSKDN

SEQ ID NO: 644 - protein, 6 aa, Artificial Sequence
  Synthetic peptide
CGSKDN

SEQ ID NO: 645 - protein, 5 aa, Artificial Sequence
  Synthetic peptide
GSKDN

SEQ ID NO: 646 - protein, 4 aa, Artificial Sequence
  Synthetic peptide
SKDN

SEQ ID NO: 647 - protein, 10 aa, Artificial Sequence
  Synthetic peptide
QSKCGSKDNI

SEQ ID NO: 648 - protein, 9 aa, Artificial Sequence
  Synthetic peptide
SKCGSKDNI

SEQ ID NO: 649 - protein, 8 aa, Artificial Sequence
  Synthetic peptide
KCGSKDNI

SEQ ID NO: 650 - protein, 7 aa, Artificial Sequence
  Synthetic peptide
CGSKDNI

SEQ ID NO: 651 - protein, 6 aa, Artificial Sequence
  Synthetic peptide
GSKDNI

SEQ ID NO: 652 - protein, 5 aa, Artificial Sequence
  Synthetic peptide
SKDNI

SEQ ID NO: 653 - protein, 8 aa, Artificial Sequence
  Synthetic peptide
GSKDNIKH

SEQ ID NO: 654 - protein, 9 aa, Artificial Sequence
  Synthetic peptide
GSKDNIKHV

SEQ ID NO: 655 - protein, 10 aa, Artificial Sequence
  Synthetic peptide
GSKDNIKHVP

SEQ ID NO: 656 - protein, 10 aa, Artificial Sequence
  Synthetic peptide
SKVTSKCGSL

SEQ ID NO: 657 - protein, 9 aa, Artificial Sequence
  Synthetic peptide
KVTSKCGSL

SEQ ID NO: 658 - protein, 8 aa, Artificial Sequence
  Synthetic peptide
VTSKCGSL

SEQ ID NO: 659 - protein, 7 aa, Artificial Sequence
  Synthetic peptide
TSKCGSL

SEQ ID NO: 660 - protein, 6 aa, Artificial Sequence
  Synthetic peptide
SKCGSL

SEQ ID NO: 661 - protein, 5 aa, Artificial Sequence
  Synthetic peptide
KCGSL

SEQ ID NO: 662 - protein, 4 aa, Artificial Sequence
  Synthetic peptide
CGSL

SEQ ID NO: 663 - protein, 3 aa, Artificial Sequence
  Synthetic peptide
GSL

SEQ ID NO: 664 - protein, 10 aa, Artificial Sequence
  Synthetic peptide
KVTSKCGSLG

SEQ ID NO: 665 - protein, 9 aa, Artificial Sequence
  Synthetic peptide
VTSKCGSLG

SEQ ID NO: 666 - protein, 8 aa, Artificial Sequence
  Synthetic peptide
TSKCGSLG

SEQ ID NO: 667 - protein, 7 aa, Artificial Sequence
  Synthetic peptide
SKCGSLG

SEQ ID NO: 668 - protein, 6 aa, Artificial Sequence
  Synthetic peptide
KCGSLG

SEQ ID NO: 669 - protein, 5 aa, Artificial Sequence
  Synthetic peptide
CGSLG

SEQ ID NO: 670 - protein, 4 aa, Artificial Sequence
  Synthetic peptide
GSLG

SEQ ID NO: 671 - protein, 3 aa, Artificial Sequence
  Synthetic peptide
SLG

SEQ ID NO: 672 - protein, 10 aa, Artificial Sequence
  Synthetic peptide
VTSKCGSLGN

SEQ ID NO: 673 - protein, 9 aa, Artificial Sequence
  Synthetic peptide
TSKCGSLGN

SEQ ID NO: 674 - protein, 8 aa, Artificial Sequence
  Synthetic peptide
SKCGSLGN

SEQ ID NO: 675 - protein, 7 aa, Artificial Sequence
  Synthetic peptide
KCGSLGN

SEQ ID NO: 676 - protein, 6 aa, Artificial Sequence
  Synthetic peptide
CGSLGN

SEQ ID NO: 677 - protein, 5 aa, Artificial Sequence
  Synthetic peptide
GSLGN

SEQ ID NO: 678 - protein, 4 aa, Artificial Sequence
  Synthetic peptide
SLGN

SEQ ID NO: 679 - protein, 10 aa, Artificial Sequence
  Synthetic peptide
TSKCGSLGNI

SEQ ID NO: 680 - protein, 9 aa, Artificial Sequence
  Synthetic peptide
SKCGSLGNI

SEQ ID NO: 681 - protein, 8 aa, Artificial Sequence
  Synthetic peptide
KCGSLGNI

SEQ ID NO: 682 - protein, 7 aa, Artificial Sequence
  Synthetic peptide
CGSLGNI

SEQ ID NO: 683 - protein, 6 aa, Artificial Sequence
  Synthetic peptide
GSLGNI

SEQ ID NO: 684 - protein, 5 aa, Artificial Sequence
  Synthetic peptide
SLGNI

SEQ ID NO: 685 - protein, 10 aa, Artificial Sequence
  Synthetic peptide
SKCGSLGNIH

SEQ ID NO: 686 - protein, 9 aa, Artificial Sequence
  Synthetic peptide
KCGSLGNIH

SEQ ID NO: 687 - protein, 8 aa, Artificial Sequence
  Synthetic peptide
CGSLGNIH

SEQ ID NO: 688 - protein, 7 aa, Artificial Sequence
  Synthetic peptide
GSLGNIH

SEQ ID NO: 689 - protein, 6 aa, Artificial Sequence
  Synthetic peptide
SLGNIH

SEQ ID NO: 690 - protein, 10 aa, Artificial Sequence
  Synthetic peptide
KCGSLGNIHH

SEQ ID NO: 691 - protein, 9 aa, Artificial Sequence
  Synthetic peptide
CGSLGNIHH

SEQ ID NO: 692 - protein, 8 aa, Artificial Sequence
  Synthetic peptide
GSLGNIHH

SEQ ID NO: 693 - protein, 7 aa, Artificial Sequence
  Synthetic peptide
SLGNIHH

SEQ ID NO: 694 - protein, 10 aa, Artificial Sequence
  Synthetic peptide
CGSLGNIHHK

SEQ ID NO: 695 - protein, 9 aa, Artificial Sequence
  Synthetic peptide
GSLGNIHHK

SEQ ID NO: 696 - protein, 8 aa, Artificial Sequence
  Synthetic peptide
SLGNIHHK

SEQ ID NO: 697 - protein, 10 aa, Artificial Sequence
  Synthetic peptide
GSLGNIHHKP

SEQ ID NO: 698 - protein, 9 aa, Artificial Sequence
  Synthetic peptide
SLGNIHHKP

SEQ ID NO: 699 - protein, 10 aa, Artificial Sequence
  Synthetic peptide
SLGNIHHKPG

SEQ ID NO: 700 - protein, 10 aa, Artificial Sequence
  Synthetic peptide
DRVQSKIGSL

SEQ ID NO: 701 - protein, 9 aa, Artificial Sequence
  Synthetic peptide
RVQSKIGSL

SEQ ID NO: 702 - protein, 8 aa, Artificial Sequence
  Synthetic peptide
VQSKIGSL

SEQ ID NO: 703 - protein, 7 aa, Artificial Sequence
  Synthetic peptide
QSKIGSL

SEQ ID NO: 704 - protein, 6 aa, Artificial Sequence
  Synthetic peptide
SKIGSL

SEQ ID NO: 705 - protein, 5 aa, Artificial Sequence
  Synthetic peptide
KIGSL

SEQ ID NO: 706 - protein, 4 aa, Artificial Sequence
  Synthetic peptide
IGSL

SEQ ID NO: 707 - protein, 10 aa, Artificial Sequence
  Synthetic peptide
RVQSKIGSLD

SEQ ID NO: 708 - protein, 9 aa, Artificial Sequence
  Synthetic peptide
VQSKIGSLD

SEQ ID NO: 709 - protein, 8 aa, Artificial Sequence
  Synthetic peptide
QSKIGSLD

SEQ ID NO: 710 - protein, 7 aa, Artificial Sequence
  Synthetic peptide
SKIGSLD

SEQ ID NO: 711 - protein, 6 aa, Artificial Sequence
  Synthetic peptide
KIGSLD

SEQ ID NO: 712 - protein, 5 aa, Artificial Sequence
  Synthetic peptide
IGSLD

SEQ ID NO: 713 - protein, 4 aa, Artificial Sequence
  Synthetic peptide
GSLD

SEQ ID NO: 714 - protein, 3 aa, Artificial Sequence
  Synthetic peptide
SLD

SEQ ID NO: 715 - protein, 10 aa, Artificial Sequence
  Synthetic peptide
VQSKIGSLDN

SEQ ID NO: 716 - protein, 9 aa, Artificial Sequence
  Synthetic peptide
QSKIGSLDN

SEQ ID NO: 717 - protein, 8 aa, Artificial Sequence
  Synthetic peptide
SKIGSLDN

SEQ ID NO: 718 - protein, 7 aa, Artificial Sequence
  Synthetic peptide
KIGSLDN

SEQ ID NO: 719 - protein, 6 aa, Artificial Sequence
  Synthetic peptide
IGSLDN

SEQ ID NO: 720 - protein, 5 aa, Artificial Sequence
  Synthetic peptide
GSLDN

SEQ ID NO: 721 - protein, 4 aa, Artificial Sequence
  Synthetic peptide
SLDN

SEQ ID NO: 722 - protein, 10 aa, Artificial Sequence
  Synthetic peptide
QSKIGSLDNI

SEQ ID NO: 723 - protein, 9 aa, Artificial Sequence
  Synthetic peptide
SKIGSLDNI

SEQ ID NO: 724 - protein, 8 aa, Artificial Sequence
  Synthetic peptide
KIGSLDNI

SEQ ID NO: 725 - protein, 7 aa, Artificial Sequence
  Synthetic peptide
IGSLDNI

SEQ ID NO: 726 - protein, 6 aa, Artificial Sequence
  Synthetic peptide
GSLDNI

SEQ ID NO: 727 - protein, 10 aa, Artificial Sequence
  Synthetic peptide
SKIGSLDNIT

SEQ ID NO: 728 - protein, 9 aa, Artificial Sequence
  Synthetic peptide
KIGSLDNIT

SEQ ID NO: 729 - protein, 8 aa, Artificial Sequence
  Synthetic peptide
IGSLDNIT

SEQ ID NO: 730 - protein, 7 aa, Artificial Sequence
  Synthetic peptide
GSLDNIT

SEQ ID NO: 731 - protein, 6 aa, Artificial Sequence
  Synthetic peptide
SLDNIT

SEQ ID NO: 732 - protein, 10 aa, Artificial Sequence
  Synthetic peptide
KIGSLDNITH

SEQ ID NO: 733 - protein, 9 aa, Artificial Sequence
  Synthetic peptide
IGSLDNITH

SEQ ID NO: 734 - protein, 8 aa, Artificial Sequence
  Synthetic peptide
GSLDNITH

SEQ ID NO: 735 - protein, 7 aa, Artificial Sequence
  Synthetic peptide
SLDNITH

SEQ ID NO: 736 - protein, 10 aa, Artificial Sequence
  Synthetic peptide
IGSLDNITHV

SEQ ID NO: 737 - protein, 9 aa, Artificial Sequence
  Synthetic peptide
GSLDNITHV

SEQ ID NO: 738 - protein, 8 aa, Artificial Sequence
  Synthetic peptide
SLDNITHV

SEQ ID NO: 739 - protein, 10 aa, Artificial Sequence
  Synthetic peptide
GSLDNITHVP

SEQ ID NO: 740 - protein, 9 aa, Artificial Sequence
  Synthetic peptide
SLDNITHVP

SEQ ID NO: 741 - protein, 10 aa, Artificial Sequence
  Synthetic peptide
SLDNITHVPG

SEQ ID NO: 742 - protein, 10 aa, Artificial Sequence
  Synthetic peptide
  MISC_FEATURE (2)..(2): Xaa can be I or C
  MISC_FEATURE (3)..(3): Xaa can be G
  MISC_FEATURE (5)..(5): Xaa can be T or K or L
  MISC_FEATURE (6)..(6): Xaa can be E or D or G
  MISC_FEATURE (8)..(8): Xaa can be L or I
  MISC_FEATURE (9)..(9): Xaa can be K or H or T
KXXSXXNXXH

SEQ ID NO: 743 - protein, 4 aa, Artificial Sequence
  Synthetic peptide
RVRR

SEQ ID NO: 744 - protein, 4 aa, Artificial Sequence
  Synthetic peptide
GAGA

SEQ ID NO: 745 - protein, 4 aa, Artificial Sequence
  Synthetic peptide
AGAG

SEQ ID NO: 746 - protein, 4 aa, Artificial Sequence
  Synthetic peptide
KGKG

SEQ ID NO: 747 - protein, 10 aa, Artificial Sequence
  Synthetic peptide
  MISC_FEATURE (2)..(2): Xaa can be any amino acid
  MISC_FEATURE (3)..(3): Xaa can be any amino acid
  MISC_FEATURE (5)..(5): Xaa can be any amino acid
  MISC_FEATURE (6)..(6): Xaa can be any amino acid
  MISC_FEATURE (8)..(8): Xaa can be any amino acid
  MISC_FEATURE (9)..(9): Xaa can be K or H
KXXSXXNXXH

SEQ ID NO: 748 - protein, 6 aa, Artificial Sequence
  Synthetic peptide
  MISC_FEATURE (1)..(1): Xaa cna be Gln or Glu
  MISC_FEATURE (6)..(6): Xaa cna be Ser or Pro
XIVYKX

SEQ ID NO: 749 - protein, 5 aa, Artificial Sequence
  Synthetic peptide
  MISC_FEATURE (3)..(3): Xaa can be I or C
SKXGS

SEQ ID NO: 750 - protein, 758 aa, Artificial Sequence
  Synthetic peptide
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 751 - protein, 31 aa, Artificial Sequence
  Synthetic peptide
QTAPVPMPDLKNVKSKIGSTENLKHQPGGGK

SEQ ID NO: 752 - protein, 31 aa, Artificial Sequence
  Synthetic peptide
VQIINKKLDLSNVQSKCGSKDNIKHVPGGGS

SEQ ID NO: 753 - protein, 31 aa, Artificial Sequence
  Synthetic peptide
VQIVYKPVDLSKVTSKCGSLGNIHHKPGGGQ

SEQ ID NO: 754 - protein, 32 aa, Artificial Sequence
  Synthetic peptide
VEVKSEKLDFKDRVQSKIGSLDNITHVPGGGN

SEQ ID NO: 755 - protein, 8 aa, Artificial Sequence
  Synthetic peptide
PDLKNVKS

SEQ ID NO: 756 - protein, 8 aa, Artificial Sequence
  Synthetic peptide
DLKNVKSK

SEQ ID NO: 757 - protein, 8 aa, Artificial Sequence
  Synthetic peptide
LKNVKSKI

SEQ ID NO: 758 - protein, 8 aa, Artificial Sequence
  Synthetic peptide
KNVKSKIG

SEQ ID NO: 759 - protein, 8 aa, Artificial Sequence
  Synthetic peptide
NVKSKIGS

SEQ ID NO: 760 - protein, 8 aa, Artificial Sequence
  Synthetic peptide
LDLSNVQS

SEQ ID NO: 761 - protein, 8 aa, Artificial Sequence
  Synthetic peptide
DLSNVQSK

SEQ ID NO: 762 - protein, 8 aa, Artificial Sequence
  Synthetic peptide
LSNVQSKC

SEQ ID NO: 763 - protein, 8 aa, Artificial Sequence
  Synthetic peptide
SNVQSKCG

SEQ ID NO: 764 - protein, 8 aa, Artificial Sequence
  Synthetic peptide
NVQSKCGS

SEQ ID NO: 765 - protein, 8 aa, Artificial Sequence
  Synthetic peptide
VDLSKVTS

SEQ ID NO: 766 - protein, 8 aa, Artificial Sequence
  Synthetic peptide
DLSKVTSK

SEQ ID NO: 767 - protein, 8 aa, Artificial Sequence
  Synthetic peptide
LSKVTSKC

SEQ ID NO: 768 - protein, 8 aa, Artificial Sequence
  Synthetic peptide
SKVTSKCG

SEQ ID NO: 769 - protein, 8 aa, Artificial Sequence
  Synthetic peptide
KVTSKCGS

SEQ ID NO: 770 - protein, 8 aa, Artificial Sequence
  Synthetic peptide
VTSKCGSL

SEQ ID NO: 771 - protein, 8 aa, Artificial Sequence
  Synthetic peptide
LDFKDRVQ

SEQ ID NO: 772 - protein, 8 aa, Artificial Sequence
  Synthetic peptide
DFKDRVQS

SEQ ID NO: 773 - protein, 8 aa, Artificial Sequence
  Synthetic peptide
FKDRVQSK

SEQ ID NO: 774 - protein, 8 aa, Artificial Sequence
  Synthetic peptide
KDRVQSKI

SEQ ID NO: 775 - protein, 8 aa, Artificial Sequence
  Synthetic peptide
DRVQSKIG

SEQ ID NO: 776 - protein, 8 aa, Artificial Sequence
  Synthetic peptide
RVQSKIGS

SEQ ID NO: 777 - protein, 12 aa, Artificial Sequence
  Synthetic peptide
VKSKIGSTEGGC

SEQ ID NO: 778 - protein, 11 aa, Artificial Sequence
  Synthetic peptide
KSKIGSTEGGC

SEQ ID NO: 779 - protein, 11 aa, Artificial Sequence
  Synthetic peptide
SKIGSTENGGC

SEQ ID NO: 780 - protein, 11 aa, Artificial Sequence
  Synthetic peptide
KIGSTENLGGC

SEQ ID NO: 781 - protein, 11 aa, Artificial Sequence
  Synthetic peptide
IGSTENLKGGC

SEQ ID NO: 782 - protein, 11 aa, Artificial Sequence
  Synthetic peptide
GSTENLKHGGC

SEQ ID NO: 783 - protein, 11 aa, Artificial Sequence
  Synthetic peptide
STENLKHQGGC

SEQ ID NO: 784 - protein, 11 aa, Artificial Sequence
  Synthetic peptide
TENLKHQPGGC

SEQ ID NO: 785 - protein, 11 aa, Artificial Sequence
  Synthetic peptide
ENLKHQPGGGC

SEQ ID NO: 786 - protein, 11 aa, Artificial Sequence
  Synthetic peptide
VKSKIGSTGGC

SEQ ID NO: 787 - protein, 11 aa, Artificial Sequence
  Synthetic peptide
PDLKNVKSGGC

SEQ ID NO: 788 - protein, 11 aa, Artificial Sequence
  Synthetic peptide
DLKNVKSKGGC

SEQ ID NO: 789 - protein, 11 aa, Artificial Sequence
  Synthetic peptide
LKNVKSKIGGC

SEQ ID NO: 790 - protein, 11 aa, Artificial Sequence
  Synthetic peptide
KNVKSKIGGGC

SEQ ID NO: 791 - protein, 11 aa, Artificial Sequence
  Synthetic peptide
NVKSKIGSGGC

SEQ ID NO: 792 - protein, 11 aa, Artificial Sequence
  Synthetic peptide
NLKHQPGGGGC

SEQ ID NO: 793 - protein, 11 aa, Artificial Sequence
  Synthetic peptide
LKHQPGGGGGC

SEQ ID NO: 794 - protein, 11 aa, Artificial Sequence
  Synthetic peptide
LDLSNVQSGGC

SEQ ID NO: 795 - protein, 11 aa, Artificial Sequence
  Synthetic peptide
DLSNVQSKGGC

SEQ ID NO: 796 - protein, 11 aa, Artificial Sequence
  Synthetic peptide
LSNVQSKCGGC

SEQ ID NO: 797 - protein, 11 aa, Artificial Sequence
  Synthetic peptide
SNVQSKCGGGC

SEQ ID NO: 798 - protein, 11 aa, Artificial Sequence
  Synthetic peptide
NVQSKCGSGGC

SEQ ID NO: 799 - protein, 11 aa, Artificial Sequence
  Synthetic peptide
VQSKCGSKGGC

SEQ ID NO: 800 - protein, 11 aa, Artificial Sequence
  Synthetic peptide
QSKCGSKDGGC

SEQ ID NO: 801 - protein, 11 aa, Artificial Sequence
  Synthetic peptide
SKCGSKDNGGC

SEQ ID NO: 802 - protein, 11 aa, Artificial Sequence
  Synthetic peptide
KCGSKDNIGGC

SEQ ID NO: 803 - protein, 11 aa, Artificial Sequence
  Synthetic peptide
CGSKDNIKGGC

SEQ ID NO: 804 - protein, 11 aa, Artificial Sequence
  Synthetic peptide
GSKDNIKHGGC

SEQ ID NO: 805 - protein, 11 aa, Artificial Sequence
  Synthetic peptide
SKDNIKHVGGC

SEQ ID NO: 806 - protein, 11 aa, Artificial Sequence
  Synthetic peptide
KDNIKHVPGGC

SEQ ID NO: 807 - protein, 11 aa, Artificial Sequence
  Synthetic peptide
DNIKHVPGGGC

SEQ ID NO: 808 - protein, 11 aa, Artificial Sequence
  Synthetic peptide
NIKHVPGGGGC

SEQ ID NO: 809 - protein, 11 aa, Artificial Sequence
  Synthetic peptide
IKHVPGGGGGC

SEQ ID NO: 810 - protein, 11 aa, Artificial Sequence
  Synthetic peptide
VDLSKVTSGGC

SEQ ID NO: 811 - protein, 11 aa, Artificial Sequence
  Synthetic peptide
DLSKVTSKGGC

SEQ ID NO: 812 - protein, 11 aa, Artificial Sequence
  Synthetic peptide
LSKVTSKCGGC

SEQ ID NO: 813 - protein, 11 aa, Artificial Sequence
  Synthetic peptide
SKVTSKCGGGC

SEQ ID NO: 814 - protein, 11 aa, Artificial Sequence
  Synthetic peptide
KVTSKCGSGGC

SEQ ID NO: 815 - protein, 11 aa, Artificial Sequence
  Synthetic peptide
VTSKCGSLGGC

SEQ ID NO: 816 - protein, 11 aa, Artificial Sequence
  Synthetic peptide
TSKCGSLGGGC

SEQ ID NO: 817 - protein, 11 aa, Artificial Sequence
  Synthetic peptide
SKCGSLGNGGC

SEQ ID NO: 818 - protein, 11 aa, Artificial Sequence
  Synthetic peptide
KCGSLGNIGGC

SEQ ID NO: 819 - protein, 11 aa, Artificial Sequence
  Synthetic peptide
CGSLGNIHGGC

SEQ ID NO: 820 - protein, 11 aa, Artificial Sequence
  Synthetic peptide
GSLGNIHHGGC

SEQ ID NO: 821 - protein, 11 aa, Artificial Sequence
  Synthetic peptide
SLGNIHHKGGC

SEQ ID NO: 822 - protein, 11 aa, Artificial Sequence
  Synthetic peptide
LGNIHHKPGGC

SEQ ID NO: 823 - protein, 11 aa, Artificial Sequence
  Synthetic peptide
GNIHHKPGGGC

SEQ ID NO: 824 - protein, 11 aa, Artificial Sequence
  Synthetic peptide
NIHHKPGGGGC

SEQ ID NO: 825 - protein, 11 aa, Artificial Sequence
  Synthetic peptide
IHHKPGGGGGC

SEQ ID NO: 826 - protein, 11 aa, Artificial Sequence
  Synthetic peptide
LDFKDRVQGGC

SEQ ID NO: 827 - protein, 11 aa, Artificial Sequence
  Synthetic peptide
DFKDRVQSGGC

SEQ ID NO: 828 - protein, 11 aa, Artificial Sequence
  Synthetic peptide
FKDRVQSKGGC

SEQ ID NO: 829 - protein, 11 aa, Artificial Sequence
  Synthetic peptide
KDRVQSKIGGC

SEQ ID NO: 830 - protein, 11 aa, Artificial Sequence
  Synthetic peptide
DRVQSKIGGGC

SEQ ID NO: 831 - protein, 11 aa, Artificial Sequence
  Synthetic peptide
RVQSKIGSGGC

SEQ ID NO: 832 - protein, 11 aa, Artificial Sequence
  Synthetic peptide
VQSKIGSLGGC

SEQ ID NO: 833 - protein, 11 aa, Artificial Sequence
  Synthetic peptide
QSKIGSLDGGC

SEQ ID NO: 834 - protein, 11 aa, Artificial Sequence
  Synthetic peptide
SKIGSLDNGGC

SEQ ID NO: 835 - protein, 11 aa, Artificial Sequence
  Synthetic peptide
KIGSLDNIGGC

SEQ ID NO: 836 - protein, 11 aa, Artificial Sequence
  Synthetic peptide
IGSLDNITGGC

SEQ ID NO: 837 - protein, 11 aa, Artificial Sequence
  Synthetic peptide
GSLDNITHGGC

SEQ ID NO: 838 - protein, 11 aa, Artificial Sequence
  Synthetic peptide
SLDNITHVGGC

SEQ ID NO: 839 - protein, 11 aa, Artificial Sequence
  Synthetic peptide
LDNITHVPGGC

SEQ ID NO: 840 - protein, 11 aa, Artificial Sequence
  Synthetic peptide
DNITHVPGGGC

SEQ ID NO: 841 - protein, 11 aa, Artificial Sequence
  Synthetic peptide
NITHVPGGGGC

SEQ ID NO: 842 - protein, 11 aa, Artificial Sequence
  Synthetic peptide
ITHVPGGGGGC

SEQ ID NO: 843 - protein, 13 aa, Artificial Sequence
  Synthetic peptide
VKSKIGSTEGGGC

SEQ ID NO: 844 - protein, 12 aa, Artificial Sequence
  Synthetic peptide
KSKIGSTEGGGC

SEQ ID NO: 845 - protein, 12 aa, Artificial Sequence
  Synthetic peptide
SKIGSTENGGGC

SEQ ID NO: 846 - protein, 12 aa, Artificial Sequence
  Synthetic peptide
KIGSTENLGGGC

SEQ ID NO: 847 - protein, 12 aa, Artificial Sequence
  Synthetic peptide
IGSTENLKGGGC

SEQ ID NO: 848 - protein, 12 aa, Artificial Sequence
  Synthetic peptide
GSTENLKHGGGC

SEQ ID NO: 849 - protein, 12 aa, Artificial Sequence
  Synthetic peptide
STENLKHQGGGC

SEQ ID NO: 850 - protein, 12 aa, Artificial Sequence
  Synthetic peptide
TENLKHQPGGGC

SEQ ID NO: 851 - protein, 12 aa, Artificial Sequence
  Synthetic peptide
VKSKIGSTGGGC

SEQ ID NO: 852 - protein, 12 aa, Artificial Sequence
  Synthetic peptide
PDLKNVKSGGGC

SEQ ID NO: 853 - protein, 12 aa, Artificial Sequence
  Synthetic peptide
DLKNVKSKGGGC

SEQ ID NO: 854 - protein, 12 aa, Artificial Sequence
  Synthetic peptide
LKNVKSKIGGGC

SEQ ID NO: 855 - protein, 12 aa, Artificial Sequence
  Synthetic peptide
KNVKSKIGGGGC

SEQ ID NO: 856 - protein, 12 aa, Artificial Sequence
  Synthetic peptide
NVKSKIGSGGGC

SEQ ID NO: 857 - protein, 12 aa, Artificial Sequence
  Synthetic peptide
ENLKHQPGGGGC

SEQ ID NO: 858 - protein, 12 aa, Artificial Sequence
  Synthetic peptide
NLKHQPGGGGGC

SEQ ID NO: 859 - protein, 12 aa, Artificial Sequence
  Synthetic peptide
LKHQPGGGGGGC

SEQ ID NO: 860 - protein, 12 aa, Artificial Sequence
  Synthetic peptide
LDLSNVQSGGGC

SEQ ID NO: 861 - protein, 12 aa, Artificial Sequence
  Synthetic peptide
DLSNVQSKGGGC

SEQ ID NO: 862 - protein, 12 aa, Artificial Sequence
  Synthetic peptide
LSNVQSKCGGGC

SEQ ID NO: 863 - protein, 12 aa, Artificial Sequence
  Synthetic peptide
SNVQSKCGGGGC

SEQ ID NO: 864 - protein, 12 aa, Artificial Sequence
  Synthetic peptide
NVQSKCGSGGGC

SEQ ID NO: 865 - protein, 12 aa, Artificial Sequence
  Synthetic peptide
VQSKCGSKGGGC

SEQ ID NO: 866 - protein, 12 aa, Artificial Sequence
  Synthetic peptide
QSKCGSKDGGGC

SEQ ID NO: 867 - protein, 12 aa, Artificial Sequence
  Synthetic peptide
SKCGSKDNGGGC

SEQ ID NO: 868 - protein, 12 aa, Artificial Sequence
  Synthetic peptide
KCGSKDNIGGGC

SEQ ID NO: 869 - protein, 12 aa, Artificial Sequence
  Synthetic peptide
CGSKDNIKGGGC

SEQ ID NO: 870 - protein, 12 aa, Artificial Sequence
  Synthetic peptide
GSKDNIKHGGGC

SEQ ID NO: 871 - protein, 12 aa, Artificial Sequence
  Synthetic peptide
SKDNIKHVGGGC

SEQ ID NO: 872 - protein, 12 aa, Artificial Sequence
  Synthetic peptide
KDNIKHVPGGGC

SEQ ID NO: 873 - protein, 12 aa, Artificial Sequence
  Synthetic peptide
DNIKHVPGGGGC

SEQ ID NO: 874 - protein, 12 aa, Artificial Sequence
  Synthetic peptide
NIKHVPGGGGGC

SEQ ID NO: 875 - protein, 12 aa, Artificial Sequence
  Synthetic peptide
IKHVPGGGGGGC

SEQ ID NO: 876 - protein, 12 aa, Artificial Sequence
  Synthetic peptide
VDLSKVTSGGGC

SEQ ID NO: 877 - protein, 12 aa, Artificial Sequence
  Synthetic peptide
DLSKVTSKGGGC

SEQ ID NO: 878 - protein, 12 aa, Artificial Sequence
  Synthetic peptide
LSKVTSKCGGGC

SEQ ID NO: 879 - protein, 12 aa, Artificial Sequence
  Synthetic peptide
SKVTSKCGGGGC

SEQ ID NO: 880 - protein, 12 aa, Artificial Sequence
  Synthetic peptide
KVTSKCGSGGGC

SEQ ID NO: 881 - protein, 12 aa, Artificial Sequence
  Synthetic peptide
VTSKCGSLGGGC

SEQ ID NO: 882 - protein, 12 aa, Artificial Sequence
  Synthetic peptide
TSKCGSLGGGGC

SEQ ID NO: 883 - protein, 12 aa, Artificial Sequence
  Synthetic peptide
SKCGSLGNGGGC

SEQ ID NO: 884 - protein, 12 aa, Artificial Sequence
  Synthetic peptide
KCGSLGNIGGGC

SEQ ID NO: 885 - protein, 12 aa, Artificial Sequence
  Synthetic peptide
CGSLGNIHGGGC

SEQ ID NO: 886 - protein, 12 aa, Artificial Sequence
  Synthetic peptide
GSLGNIHHGGGC

SEQ ID NO: 887 - protein, 12 aa, Artificial Sequence
  Synthetic peptide
SLGNIHHKGGGC

SEQ ID NO: 888 - protein, 12 aa, Artificial Sequence
  Synthetic peptide
LGNIHHKPGGGC

SEQ ID NO: 889 - protein, 12 aa, Artificial Sequence
  Synthetic peptide
GNIHHKPGGGGC

SEQ ID NO: 890 - protein, 12 aa, Artificial Sequence
  Synthetic peptide
NIHHKPGGGGGC

SEQ ID NO: 891 - protein, 12 aa, Artificial Sequence
  Synthetic peptide
IHHKPGGGGGGC

SEQ ID NO: 892 - protein, 12 aa, Artificial Sequence
  Synthetic peptide
LDFKDRVQGGGC

SEQ ID NO: 893 - protein, 12 aa, Artificial Sequence
  Synthetic peptide
DFKDRVQSGGGC

SEQ ID NO: 894 - protein, 12 aa, Artificial Sequence
  Synthetic peptide
FKDRVQSKGGGC

SEQ ID NO: 895 - protein, 12 aa, Artificial Sequence
  Synthetic peptide
KDRVQSKIGGGC

SEQ ID NO: 896 - protein, 12 aa, Artificial Sequence
  Synthetic peptide
DRVQSKIGGGGC

SEQ ID NO: 897 - protein, 12 aa, Artificial Sequence
  Synthetic peptide
RVQSKIGSGGGC

SEQ ID NO: 898 - protein, 12 aa, Artificial Sequence
  Synthetic peptide
VQSKIGSLGGGC

SEQ ID NO: 899 - protein, 12 aa, Artificial Sequence
  Synthetic peptide
QSKIGSLDGGGC

SEQ ID NO: 900 - protein, 12 aa, Artificial Sequence
  Synthetic peptide
SKIGSLDNGGGC

SEQ ID NO: 901 - protein, 12 aa, Artificial Sequence
  Synthetic peptide
KIGSLDNIGGGC

SEQ ID NO: 902 - protein, 12 aa, Artificial Sequence
  Synthetic peptide
IGSLDNITGGGC

SEQ ID NO: 903 - protein, 12 aa, Artificial Sequence
  Synthetic peptide
GSLDNITHGGGC

SEQ ID NO: 904 - protein, 12 aa, Artificial Sequence
  Synthetic peptide
SLDNITHVGGGC

SEQ ID NO: 905 - protein, 12 aa, Artificial Sequence
  Synthetic peptide
LDNITHVPGGGC

SEQ ID NO: 906 - protein, 12 aa, Artificial Sequence
  Synthetic peptide
DNITHVPGGGGC

SEQ ID NO: 907 - protein, 12 aa, Artificial Sequence
  Synthetic peptide
NITHVPGGGGGC

SEQ ID NO: 908 - protein, 12 aa, Artificial Sequence
  Synthetic peptide
ITHVPGGGGGGC

SEQ ID NO: 909 - protein, 11 aa, Artificial Sequence
  Synthetic peptide
SKIGSTENLKH

SEQ ID NO: 910 - protein, 11 aa, Artificial Sequence
  Synthetic peptide
SKIGSTENIKH

SEQ ID NO: 911 - protein, 11 aa, Artificial Sequence
  Synthetic peptide
SKIGSKDNLKH

SEQ ID NO: 912 - protein, 11 aa, Artificial Sequence
  Synthetic peptide
SKIGSKENIKH

SEQ ID NO: 913 - protein, 11 aa, Artificial Sequence
  Synthetic peptide
SKIGSLENLKH

SEQ ID NO: 914 - protein, 11 aa, Artificial Sequence
  Synthetic peptide
SKIGSLENIKH

SEQ ID NO: 915 - protein, 11 aa, Artificial Sequence
  Synthetic peptide
SKIGSTDNLKH

SEQ ID NO: 916 - protein, 11 aa, Artificial Sequence
  Synthetic peptide
SKIGSTDNIKH

SEQ ID NO: 917 - protein, 11 aa, Artificial Sequence
  Synthetic peptide
SKIGSKDNLKH

SEQ ID NO: 918 - protein, 11 aa, Artificial Sequence
  Synthetic peptide
SKIGSKDNIKH

SEQ ID NO: 919 - protein, 11 aa, Artificial Sequence
  Synthetic peptide
SKIGSLDNLKH

SEQ ID NO: 920 - protein, 11 aa, Artificial Sequence
  Synthetic peptide
SKIGSLDNIKH

SEQ ID NO: 921 - protein, 11 aa, Artificial Sequence
  Synthetic peptide
SKIGSTGNLKH

SEQ ID NO: 922 - protein, 11 aa, Artificial Sequence
  Synthetic peptide
SKIGSTGNIKH

SEQ ID NO: 923 - protein, 11 aa, Artificial Sequence
  Synthetic peptide
SKIGSKGNLKH

SEQ ID NO: 924 - protein, 11 aa, Artificial Sequence
  Synthetic peptide
SKIGSKGNIKH

SEQ ID NO: 925 - protein, 11 aa, Artificial Sequence
  Synthetic peptide
SKIGSLGNLKH

SEQ ID NO: 926 - protein, 11 aa, Artificial Sequence
  Synthetic peptide
SKIGSLGNIKH

SEQ ID NO: 927 - protein, 14 aa, Artificial Sequence
  Synthetic peptide
SKIGSTENLKHGGC

SEQ ID NO: 928 - protein, 14 aa, Artificial Sequence
  Synthetic peptide
SKIGSTENIKHGGC

SEQ ID NO: 929 - protein, 14 aa, Artificial Sequence
  Synthetic peptide
SKIGSKDNLKHGGC

SEQ ID NO: 930 - protein, 14 aa, Artificial Sequence
  Synthetic peptide
SKIGSKENIKHGGC

SEQ ID NO: 931 - protein, 14 aa, Artificial Sequence
  Synthetic peptide
SKIGSLENLKHGGC

SEQ ID NO: 932 - protein, 14 aa, Artificial Sequence
  Synthetic peptide
SKIGSLENIKHGGC

SEQ ID NO: 933 - protein, 14 aa, Artificial Sequence
  Synthetic peptide
SKIGSTDNLKHGGC

SEQ ID NO: 934 - protein, 14 aa, Artificial Sequence
  Synthetic peptide
SKIGSTDNIKHGGC

SEQ ID NO: 935 - protein, 14 aa, Artificial Sequence
  Synthetic peptide
SKIGSKDNLKHGGC

SEQ ID NO: 936 - protein, 14 aa, Artificial Sequence
  Synthetic peptide
SKIGSKDNIKHGGC

SEQ ID NO: 937 - protein, 14 aa, Artificial Sequence
  Synthetic peptide
SKIGSLDNLKHGGC

SEQ ID NO: 938 - protein, 14 aa, Artificial Sequence
  Synthetic peptide
SKIGSLDNIKHGGC

SEQ ID NO: 939 - protein, 14 aa, Artificial Sequence
  Synthetic peptide
SKIGSTGNLKHGGC

SEQ ID NO: 940 - protein, 14 aa, Artificial Sequence
  Synthetic peptide
SKIGSTGNIKHGGC

SEQ ID NO: 941 - protein, 14 aa, Artificial Sequence
  Synthetic peptide
SKIGSKGNLKHGGC

SEQ ID NO: 942 - protein, 14 aa, Artificial Sequence
  Synthetic peptide
SKIGSKGNIKHGGC

SEQ ID NO: 943 - protein, 14 aa, Artificial Sequence
  Synthetic peptide
SKIGSLGNLKHGGC

SEQ ID NO: 944 - protein, 14 aa, Artificial Sequence
  Synthetic peptide
SKIGSLGNIKHGGC

SEQ ID NO: 945 - protein, 15 aa, Artificial Sequence
  Synthetic peptide
SKIGSTENLKHGGGC

SEQ ID NO: 946 - protein, 15 aa, Artificial Sequence
  Synthetic peptide
SKIGSTENIKHGGGC

SEQ ID NO: 947 - protein, 15 aa, Artificial Sequence
  Synthetic peptide
SKIGSKDNLKHGGGC

SEQ ID NO: 948 - protein, 15 aa, Artificial Sequence
  Synthetic peptide
SKIGSKENIKHGGGC

SEQ ID NO: 949 - protein, 15 aa, Artificial Sequence
  Synthetic peptide
SKIGSLENLKHGGGC

SEQ ID NO: 950 - protein, 15 aa, Artificial Sequence
  Synthetic peptide
SKIGSLENIKHGGGC

SEQ ID NO: 951 - protein, 15 aa, Artificial Sequence
  Synthetic peptide
SKIGSTDNLKHGGGC

SEQ ID NO: 952 - protein, 15 aa, Artificial Sequence
  Synthetic peptide
SKIGSTDNIKHGGGC

SEQ ID NO: 953 - protein, 15 aa, Artificial Sequence
  Synthetic peptide
SKIGSKDNLKHGGGC

SEQ ID NO: 954 - protein, 15 aa, Artificial Sequence
  Synthetic peptide
SKIGSKDNIKHGGGC

SEQ ID NO: 955 - protein, 15 aa, Artificial Sequence
  Synthetic peptide
SKIGSLDNLKHGGGC

SEQ ID NO: 956 - protein, 15 aa, Artificial Sequence
  Synthetic peptide
SKIGSLDNIKHGGGC

SEQ ID NO: 957 - protein, 15 aa, Artificial Sequence
  Synthetic peptide
SKIGSTGNLKHGGGC

SEQ ID NO: 958 - protein, 15 aa, Artificial Sequence
  Synthetic peptide
SKIGSTGNIKHGGGC

SEQ ID NO: 959 - protein, 15 aa, Artificial Sequence
  Synthetic peptide
SKIGSKGNLKHGGGC

SEQ ID NO: 960 - protein, 15 aa, Artificial Sequence
  Synthetic peptide
SKIGSKGNIKHGGGC

SEQ ID NO: 961 - protein, 15 aa, Artificial Sequence
  Synthetic peptide
SKIGSLGNLKHGGGC

SEQ ID NO: 962 - protein, 15 aa, Artificial Sequence
  Synthetic peptide
SKIGSLGNIKHGGGC

SEQ ID NO: 963 - protein, 14 aa, Artificial Sequence
  Synthetic peptide
CGGSKIGSTDNIKH

SEQ ID NO: 964 - protein, 14 aa, Artificial Sequence
  Synthetic peptide
CGGSKIGSKDNIKH

SEQ ID NO: 965 - protein, 14 aa, Artificial Sequence
  Synthetic peptide
CGGSKIGSLDNIKH

SEQ ID NO: 966 - protein, 15 aa, Artificial Sequence
  Synthetic peptide
CGGGSKIGSTDNIKH

SEQ ID NO: 967 - protein, 15 aa, Artificial Sequence
  Synthetic peptide
CGGGSKIGSKDNIKH

SEQ ID NO: 968 - protein, 15 aa, Artificial Sequence
  Synthetic peptide
CGGGSKIGSLDNIKH

SEQ ID NO: 969 - protein, 4 aa, Artificial Sequence
  Synthetic peptide
GGGS

SEQ ID NO: 970 - protein, 5 aa, Artificial Sequence
  Synthetic peptide
GGGGS

(19) United States
(12) Patent Application Publication
(10) Pub. No.: US 2023/0302127 A1
(43) Pub. Date: Sep. 28, 2023

(54) TAU VACCINE FOR THE TREATMENT OF ALZHEIMER'S DISEASE

(71) Applicant: OTHAIR PROTHENA LIMITED, Dublin 2 (IE)

(72) Inventors: Robin Barbour, Walnut Creek, CA (US); Gene Kinney, Boca Raton, FL (US); Wagner Zago, San Carlos, CA (US); Tarlochan S. Nijjar, Orinda, CA (US)

(21) Appl. No.: 18/020,037

(22) PCT Filed: May 19, 2021
(86) PCT No.: PCT/US2021/033189

§ 371 Date: Feb. 6, 2023

Related U.S. Application Data
(60) Provisional application No. 63/062,971, filed on Aug. 7, 2020.

(51) Int. Cl. A61K 39/395; A61P 25/28

(52) U.S. Cl. CPC A61K 39/3955; A61P 25/28; A61K 2039/6037

(57) ABSTRACT

The disclosure provides peptides, peptide compositions, immunotherapy compositions, pharmaceutical compositions and nucleic acids comprising one or more tau peptides. The disclosure also provides methods of treating or effecting prophylaxis of Alzheimer's disease or other diseases characterized at least in part by aberrant tau pathology (e.g., aggregation in neurofibrillary tangles) in a subject, including methods of clearing deposits, inhibiting or reducing aggregation of tau, blocking the uptake by neurons, clearing tau, and inhibiting propagation of tau seeds in a subject having or at risk of developing Alzheimer's disease or other diseases containing tau accumulations. The methods include administering to such patients the compositions comprising one or more tau peptides.

### RELATED APPLICATIONS

This application claims the benefit of U.S. Provisional Patent Application No. 63/062,971, filed Aug. 7, 2020, which is incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on May 19, 2021, having the file name “20-1088-WO_Sequence-Listing_ST25.txt” and is 188 kb in size.

### FIELD

The disclosure relates to the technical fields of immunology and medicine, and in particular to the treatment of Alzheimer's disease and other diseases of protein misfolding.

### BACKGROUND

Alzheimer's disease (AD) is a progressive disease resulting in senile dementia. Broadly speaking, the disease falls into two categories: late onset, which occurs in old age (65+years) and early onset, which develops well before the senile period, i.e., between 35 and 60 years. In both types of disease, the pathology is the same but the abnormalities tend to be more severe and widespread in cases beginning at an earlier age. The disease is characterized by at least two types of lesions in the brain, neurofibrillary tangles and senile plaques. Neurofibrillary tangles are intracellular deposits of microtubule associated tau protein consisting of two filaments twisted about each other in pairs. Senile plaques (i.e., amyloid plaques) are areas of disorganized neuropil up to 150 μm across with extracellular amyloid deposits at the center which are visible by microscopic analysis of sections of brain tissue.

Tau tangles constitute abnormal fibrils measuring 10 nm in diameter occurring in pairs wound in a helical fashion with a regular periodicity of 80 nm. The tau within neurofibrillary tangles is abnormally phosphorylated (hyperphosphorylated) with phosphate groups attached to specific sites on the molecule. Severe involvement of neurofibrillary tangles is seen in the layer II neurons of the entorhinal cortex, the CA1 and subicular regions of the hippocampus, the amygdala, and the deeper layers (layers III, V, and superficial VI) of the neocortex in Alzheimer's disease. Tau pathologies are known to correlate to cognitive decline.

Accordingly, there exists the need for new therapies and reagents for the prevention or treatment of Alzheimer's disease, in particular, therapies and reagents capable of causing an immune response to the tau present in patients.

### SUMMARY

In some embodiments, disclosure is directed to a peptide comprising 3-13 amino acids from residues 244-400 of SEQ ID NO:01 or from residues 1-150 of SEQ ID NO:750. For example, the peptide may comprise an amino acid sequence of one of SEQ ID NO:02 to SEQ ID NO:19, SEQ ID NO:25 to SEQ ID NO:320, SEQ ID NO:411, SEQ ID NO:454, SEQ ID NO:456, SEQ ID NO:458 to SEQ ID NO:742, SEQ ID NO:747 to SEQ ID NO:749, or SEQ ID NO:755 to SEQ ID NO:776. In some embodiments, the peptide is from the microtubule binding region (MTBR) of tau (residues 244-372 of SEQ ID NO:01) and, as an example, comprise any one of SEQ ID NO:02 to SEQ ID NO:19, SEQ ID NO:28 to SEQ ID NO:102, SEQ ID NO:185 to SEQ ID NO:320 or SEQ ID NO:458 to SEQ ID NO:742, each optionally further comprising a C-terminal cysteine.

In some embodiments, the disclosure is directed to a peptide comprising, e.g., 3-13, 7-13, 7-10 or 8 amino acids from residues 244-400 of SEQ ID NO:01 or from residues 1-150 of SEQ ID NO:750, further comprising a C-terminal -GGC or -GGGC or an N-terminal CGG- or CGGG-. For example, the peptide can comprise an amino acid sequence of SEQ ID NO:777 to SEQ ID NO:785 or SEQ ID NO:786 to SEQ ID NO:908.

In some embodiments, the peptide may include a linker to a carrier at a C-terminal portion of the peptide or at a N-terminal portion of the peptide, which may include an amino acid sequence of, for example, AA, AAA, KK, KKK, SS, SSS AGAG, GG, GGG, GAGA, and KGKG, In addition, the linker to the carrier, if present, may include a terminal cysteine (C). As an example of a C-terminal linker, the polypeptide may include the amino acid sequence of NIKHVPG-XXC (SEQ ID NO:05), wherein XX and C are independently optional and, if present, XX can be, for example, AA, KK, SS, AGAG, GG, GAGA, or KGKG. In some embodiments, the peptide further comprises a blocked amine at the N-terminus.

In other embodiments, the disclosure is directed to an immunotherapy composition including the polypeptides of the disclosure, wherein the polypeptide may be linked to a carrier. The carrier may include serum albumins. immunoglobulin molecules, thyroglobulin, ovalbumin, tetanus toxoid (TI), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

Still further, embodiments of the disclosure are directed to a pharmaceutical compositions comprising the peptides and/or the immunotherapy compositions of the disclosure. and including at least one adjuvant. The adjuvant may be aluminum hydroxide, aluminum phosphate, aluminum sulfate, 3 De-O-acylated monophosphoryl lipid A (MPL) and synthetic analogs thereof, QS-21, QS-18, QS-17, QS-7, TQL-1055, Complete Freund's Adjuvant (CFA), Incomplete Freund's Adjuvant (IFA), oil in water emulsions (such as squalene or peanut oil), CpG, polyglutamic acid, polylysine, AddaVax™, MF59®, and combinations thereof. In addition, the formulation may include one or more of a liposomal formulation, a diluent, or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

In addition, the immunotherapy composition may include at least one pharmaceutically acceptable diluent and/or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Embodiments of the disclosure are also directed to nucleic acid sequences encoding the polypeptides and the immunotherapy compositions of the disclosure. The nucleic acids may be included in a nucleic acid immunotherapy composition including the nucleic acid and at least one adjuvant.

Still further, embodiments of the disclosure are directed to methods for treating or effecting prophylaxis of Alzheimer's disease in a subject, and methods for inhibiting or reducing aggregation of tau in a subject having or at risk of developing Alzheimer's disease. The methods include administrating to the subject an immunotherapy composition, a nucleic acids immunotherapy composition, or a pharmaceutical formulation of the disclosure.

The methods of the disclosure may include repeating the administering at least a second time, at least a third time, at least a fourth time, at least a fifth time, or at least a sixth time, and may include repeating the administering at an interval of about bimonthly, of about 21 to about 28 days, of about quarterly, of about biannually, or of about annually.

Still further, methods of the disclosure are directed to inducing an immune response in an animal. The methods include administering to the animal a polypeptide, an immunotherapy composition, a pharmaceutical formulation or a nucleic acid immunotherapy composition of the disclosure in a regimen effective to generate an immune response including antibodies that specifically bind to tau. The immune response may include antibodies that specifically bind to the microtubule region of tau.

In other embodiments, the disclosure is directed to an immunization kit including an immunotherapy composition of the disclosure and may include an adjuvant, wherein the immunotherapy composition may be in a first container and the adjuvant may be a second container.

Still further, the disclosure is directed to a kit including a nucleic acid immunotherapy composition of the disclosure and may include an adjuvant. The nucleic acid may be in a first container and the adjuvant may be in a second container.

In each of the embodiments of the peptide described herein, the peptide may comprise, consist, or consist essentially of the recited sequences.

### BRIEF DESCRIPTION OF THE FIGURES

FIG. 1 shows the results of an experiment comparing the titers of Guinea pig serum for tau single peptide immunogens AGHVTQAR (SEQ ID NO:453), GYTMHQD (SEQ ID NO:454), QIVYKPV (SEQ ID NO:02) and EIVYKSPV (SEQ ID NO:141). All immunogens further comprised a C-terminal linker of GG and a cysteine for coupling to maleimide activated CRM197 carrier. QS21 was utilized as an adjuvant in AddaVax squalene-based oil-in-water nano-emulsion.

FIG. 2 shows the results of an experiment measuring the titer of murine serum for tau single peptide immunogen CNIKHVPG (SEQ ID NO:24). The peptide was coupled to maleimide activated CRM197 carrier through the N-terminal cysteine. QS21 was used as an adjuvant.

FIG. 3 shows the results of an experiment measuring the titer of murine serum for tau single peptide immunogens described by SEQ ID NO:777 to SEQ ID NO:785 and SEQ ID NO:963 to SEQ ID NO:965.

FIG. 4 shows the results of an experiment measuring the titer of murine serum for tau single peptide immunogens described by SEQ ID NO:963, SEQ ID NO:964 and SEQ ID NO:965.

FIG. 5(A)-(H) shows the results of an experiment measuring the binding of various murine sera from animals vaccinated with immunogenic compositions of the disclosure against MTBR1, MTBR2, MTBR3 and MTBR4.

FIG. 6 shows the results of an experiment measuring the ability of mouse serum with antibodies raised against VKSKIGSTEGGC (SEQ ID NO:777) to block tau binding to heparin as a potential surrogate marker for the ability of the serum to block uptake of tau into cells. For FIGS. 6-12, filled circles (“neg”) are a negative control. Samples labels, e.g., “1.1”, “1.2”, “1.3” and “1.4” in FIG. 6, refer to the peptide construct number (“1”), followed by a period, and a second number, which represents an animal. Thus, FIG. 6 illustrates the results of experiments on four mice using construct 1, which corresponds to SEQ ID NO:777.

FIG. 7 shows the results of an experiment measuring the ability of mouse serum with antibodies raised against KSKIGSTEGGC (SEQ ID NO:778) to block tau binding to heparin as a potential surrogate marker for the ability of the serum to block uptake of tau into cells.

FIG. 8 shows the results of an experiment measuring the ability of mouse serum with antibodies raised against SKIGSTENGGC (SEQ ID NO:779) to block tau binding to heparin as a potential surrogate marker for the ability of the serum to block uptake of tau into cells.

FIG. 9 shows the results of an experiment measuring the ability of mouse serum with antibodies raised against STENLKHQGGC (SEQ ID NO:783) to block tau binding to heparin as a potential surrogate marker for the ability of the serum to block uptake of tau into cells.

FIG. 10 shows the results of an experiment measuring the ability of mouse serum with antibodies raised against TENLKHQPGGC (SEQ ID NO:784) to block tau binding to heparin as a potential surrogate marker for the ability of the serum to block uptake of tau into cells.

FIG. 11 shows the results of an experiment measuring the ability of mouse serum with antibodies raised against ENLKHQPGGGC (SEQ ID NO:785) to block tau binding to heparin as a potential surrogate marker for the ability of the serum to block uptake of tau into cells.

FIG. 12 shows the results of an experiment measuring the ability of mouse serum with antibodies raised against CGGSKIGSKDNIKH (SEQ ID NO:964) to block tau binding to heparin as a potential surrogate marker for the ability of the serum to block uptake of tau into cells.

FIG. 13 shows the results of an experiment measuring the ability of mouse serum with antibodies raised against CGGSKIGSLDNIKH (SEQ ID NO:965) to block tau binding to heparin as a potential surrogate marker for the ability of the serum to block uptake of tau into cells.

FIG. 14 shows staining of Tau pathology in fresh frozen human AD brain tissue (versus normal tissue in the right side panel) using a 1:500 dilution of serum from mice vaccinated with SEQ ID NO:778.

FIG. 15 shows staining of Tau pathology in fresh frozen human AD brain tissue (versus normal tissue in the right side panel) using a 1:500 dilution of serum from mice vaccinated with (SEQ ID NO:779).

FIG. 16 shows staining of Tau pathology in fresh frozen human AD brain tissue (versus normal tissue in the right side panel) using a 1:500 dilution of serum from mice vaccinated with (SEQ ID NO:784).

FIG. 17 shows staining of Tau pathology in fresh frozen human AD brain tissue (versus normal tissue in the right side panel) using a 1:500 dilution of serum from mice vaccinated with (SEQ ID NO:785).

FIG. 18 shows staining of Tau pathology in fresh frozen human AD brain tissue (versus normal tissue in the right side panel) using a 1:500 dilution of serum from mice vaccinated with (SEQ ID NO:918).

### DESCRIPTION

The disclosure provides peptide compositions and immunotherapy compositions comprising one or more tau peptides. The disclosure also provides methods of treating or effecting prophylaxis of Alzheimer's disease or other diseases characterized at least in part by aberrant tau pathology (e.g., aggregation in neurofibrillary tangles) in a subject, including methods of clearing and preventing formation of deposits and aggregates, inhibiting or reducing aggregation of tau, blocking the binding and/or uptake of tau by neurons, inhibiting transmission of tau species between cells, and inhibiting propagation of pathology between brain regions in a subject having or at risk of developing Alzheimer's disease or other diseases containing tau accumulations. The methods include administering to such patients the compositions comprising an one or more tau peptides.

A number of terms are defined below. As used herein, the singular forms “a,” “an”, and “the” include plural referents unless the context clearly dictates otherwise. For example, the term “a compound” or “at least one compound” can include a plurality of compounds, including mixtures thereof.

Unless otherwise apparent from the context, the term “about” encompasses insubstantial variations, such as values within a standard margin of error of measurement (e.g., SEM) of a stated value. For example, the term “about” as used herein when referring to a measurable value such as a parameter, an amount, a temporal duration, can encompass variations of +/−10% or less, +/−5% or less, or +/−1% or less or less of and from the specified value. Designation of a range of values includes all integers within or defining the range, and all subranges defined by integers within the range. As used herein, statistical significance means p≤0.05.

Compositions or methods “comprising” or “including” one or more recited elements may include other elements not specifically recited. For example, a composition that “comprises” or “includes” a polypeptide sequence may contain the sequence alone or in combination with other sequences or ingredients.

An individual is at increased risk of a disease if the subject has at least one known risk-factor (e.g., age, genetic, biochemical, family history, and situational exposure) placing individuals with that risk factor at a statistically significant greater risk of developing the disease than individuals without the risk factor.

The term “patient” includes human and other mammalian subjects that receive either prophylactic or therapeutic treatment, including treatment naïve subjects. As used herein, the terms “subject” or “patient” refer to any single subject for which treatment is desired, including other mammalian subjects such as, humans, cattle, dogs, guinea pigs, rabbits, and so on. Also intended to be included as a subject are any subjects involved in clinical research trials not showing any clinical sign of disease, or subjects involved in epidemiological studies, or subjects used as controls.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, such as altered gait, as perceived by the subject. A “sign” refers to objective evidence of a disease as observed by a physician.

As used herein, the terms “treat” and “treatment” refer to the alleviation or amelioration of one or more symptoms or effects associated with the disease, prevention, inhibition or delay of the onset of one or more symptoms or effects of the disease, lessening of the severity or frequency of one or more symptoms or effects of the disease, and/or increasing or trending toward desired outcomes as described herein.

The terms “prevention”, “prevent”, or “preventing” as used herein refer to contacting (for example, administering) the peptide(s) or immunotherapy compositions of the present disclosure with a subject before the onset of a disease, with or without tau pathology already present (primary and secondary prevention), thereby delaying the onset of clinical symptoms and/or alleviating symptoms of the disease after the onset of the disease, compared to when the subject is not contacted with the peptide or immunotherapy compositions, and does not refer to completely suppressing the onset of the disease. In some cases, prevention may occur for limited time after administration of the peptide or immunotherapy compositions of the present disclosure. In other cases, prevention may occur for the duration of a treatment regimen comprising administering the peptide or immunotherapy compositions of the present disclosure.

The terms “reduction”, “reduce”, or “reducing” as used herein refer to decreasing the amount of tau present in a subject or in tissue of the subject, or suppressing an increase in the amount of tau present in a subject or in tissue of a subject, which encompasses decreasing or suppressing an increase in (e.g., decreasing the rate of increase) the amount of tau present, accumulated, aggregated, or deposited in the subject or tissue in the subject. In certain embodiments, the decrease in or suppression of an increase in (e.g., decreasing the rate of increase) the amount of tau present, accumulated, aggregated, or deposited in the subject refers to an amount of tau present, accumulated, aggregated, or deposited in the central nervous system (CNS) of the subject. In certain embodiments, the decrease in or suppression of an increase in (e.g., decreasing the rate of increase) the amount of tau present, accumulated, aggregated, or deposited in the subject refers to an amount of tau present, accumulated, aggregated, or deposited in the periphery (e.g., peripheral circulatory system) of the subject. In certain embodiments, the decrease in or suppression of an increase in (e.g., decreasing the rate of increase) the amount of tau present, accumulated, aggregated, or deposited in the subject refers to an amount of tau present, accumulated, aggregated, or deposited in the brain of the subject. In some embodiments, the tau reduced is the pathological form(s) of tau (e.g., neurofibrillary tangles of tau, dystrophic neurites). In yet other embodiment, pathological indicators of neurodegenerative disease and/or tauopathies are decreased.

The terms “epitope” or “antigenic determinant” refers to a site on an antigen to which B and/or T cells respond, or to a site on an antigen to which an antibody binds. Epitopes can be formed both from contiguous amino acids or from noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, at least 4, at least 5, at least 6, at least 7, at least 8, at least 9, at least 10, at least 11, at least 12, or at least 13 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996).

An “immunogenic agent” or “immunogen” or “antigen” is capable of inducing an immunological response against itself or modified/processed versions of itself upon administration to an animal, optionally in conjunction with an adjuvant. The terms “immunogenic agent” or “immunogen” or “antigen” refer to a compound or composition comprising a peptide, polypeptide or protein which is “antigenic” or “immunogenic” when administered in an appropriate amount (an “immunogenically effective amount”), i.e., capable of inducing, eliciting, augmenting or boosting a cellular and/or humoral immune response and of being recognized by the products of that response (T cells, antibodies). An immunogen can be a peptide, or a combination of two or more same or different peptides, that includes at least 3, at least 4, at least 5, at least 6, at least 7, at least 8, at least 9, at least 10, at least 11, at least 12, or at least 13 amino acids in a liner or spatial conformation.

An immunogen may be effective when given alone or in combination, or linked to, or fused to, another substance (which can be administered at one time or over several intervals). An immunogenic agent or immunogen may include an antigenic peptide or polypeptide that is linked to a carrier as described herein.

A nucleic acid such as DNA or RNA that encodes an antigenic peptide or polypeptide is referred to as a “DNA [or RNA] immunogen,” as the encoded peptide or polypeptide is expressed in vivo after administration of the DNA or RNA. The peptide or polypeptide can be recombinantly expressed from a vaccine vector, which can be naked DNA or RNA that comprises the peptide or polypeptide coding sequence operably linked to a promoter, e.g., an expression vector or cassette as described herein.

The term “adjuvant” refers to a compound that, when administered in conjunction with an antigen, augments the immune response to the antigen, but when administered alone does not generate an immune response to the antigen. Adjuvants can augment an immune response by several mechanisms including lymphocyte recruitment, stimulation of B and/or T cells, and stimulation of macrophages. An adjuvant may be a natural compound, a modified version of or derivative of a natural compound, or a synthetic compound.

The terms “peptide” and “polypeptide” are used interchangeably herein and refer to a chain of two or more consecutive amino acids. If and when a distinction is made, context makes the meaning clear. For example, if two or more peptides described herein are joined to make a dimeric or multimeric peptide, polypeptide may be used to indicate “poly” or “more than one” peptide.

The term “pharmaceutically acceptable” means that the carrier, diluent, excipient, adjuvant, or auxiliary is compatible with the other ingredients of a pharmaceutical formulation and not substantially deleterious to the recipient thereof.

The terms “immunotherapy” or “immune response” refer to the development of a beneficial humoral (antibody mediated) and/or a cellular (mediated by antigen-specific T cells or their secretion products) response directed against a tau peptide in a recipient. Such a response can be an active response induced by administration of immunogen (e.g. tau peptide(s)). A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

Tau

Tau is a protein with a molecular weight of about 50,000 that is normally present in nerve axons, or the like, and contributes to microtubular stability. The tau proteins (or t proteins) are a group of six highly-soluble protein isoforms produced by alternative splicing from the gene MAPT (microtubule-associated protein tau). They have roles primarily in maintaining the stability of microtubules in axons and are abundant in the neurons of the central nervous system (CNS). They are less common elsewhere but are also expressed at very low levels in CNS astrocytes and oligodendrocytes. Pathologies and dementias of the nervous system such as Alzheimer's disease and Parkinson's disease are associated with tau proteins that have become hyperphosphorylated insoluble aggregates called neurofibrillary tangles. Pathogenic tau species causes toxic effects through direct binding to cells and/or accumulation inside cells and/or initiation of misfolding processes (seeding) and can be propagated from one cell to another via cell-to-cell transmission. Toxicity could also happen by neurofibrillary tangles (NFTs), which leads to cell death and cognitive decline. Other tauopathies include, for example, progressive supranuclear palsy, corticobasal syndrome, some frontotemporal dementias, and chronic traumatic encephalopathy.

Peptide Immunogens

An agent used for active immunization can induce in a patient an immune response and can serve as an immunotherapy. Agents used for active immunization can be, for example, the same types of immunogens used for generating monoclonal antibodies in laboratory animals, and may include 3, 4, 5, 6, 7, 8, 9, 10, 11, 12, 13 or more contiguous amino acids from a region of tau peptide.

In some embodiments of the disclosure, the immunogen can comprise, consists of, or consists essentially of, a tau peptide comprising 3-13 (e.g., 7-13, 5-10, 7-11, 8) amino acids from residues 244-400 of the long form of tau (SEQ ID NO:01).

residues 1-150 of full-length tau (SEQ ID NO:750). In some embodiments, the fragment is unphosphorylated. In some embodiments, the fragment is phosphorylated at serine (S), threonine (T), and/or tyrosine (Y) phosphorylation sites.

In some embodiments, the immunogen comprises, consists of, or consists essentially of, an amino acid sequence represented by the consensus motif (Q/E)IVYK(S/P) (SEQ ID NO:748). In some embodiments, the immunogen comprises an amino acid sequence represented by the consensus motif KXXSXXNX(K/H)H (SEQ ID NO:747) where X is any amino acid. In some embodiments, the immunogen comprises an amino acid sequence represented by the consensus motif SK(I/C)GS (SEQ ID NO:749).

In some embodiments, the tau peptide comprises, consists of, or consists essentially of, an amino acid sequence selected from the group consisting of any one of SEQ ID NO:02 to SEQ ID NO:19, SEQ ID NO:25 to SEQ ID NO:320, SEQ ID NO:128, SEQ ID NO:411, SEQ ID NO:454, SEQ ID NO:456, SEQ ID NO:458 to SEQ ID NO:742, SEQ ID NO:747 to SEQ ID NO:749, or SEQ ID NO:755 to SEQ ID NO:776. In some embodiments, the immunogen comprises a tau peptide from the microtubule binding region (MTBR) of tau (residues 244-372 of SEQ ID NO:01). In some embodiment, the peptide comprises an amino acid sequence selected from the group consisting of any one of SEQ ID NO:02 to SEQ ID NO:19. SEQ ID NO:28 to SEQ ID NO:102, SEQ ID NO:185 to SEQ ID NO:320, SEQ ID NO:458 to SEQ ID NO:742, or SEQ ID NO:747 to SEQ ID NO:749. In some embodiments, the immunogen can comprise, consists of, or consists essentially of, an amino acid sequence selected from the group consisting of

(SEQ ID NO: 02)
QIVYKPV,
(SEQ ID NO: 03)
QIVYKP,
(SEQ ID NO: 04)
NIKHVP,
(SEQ ID NO: 05)
NIKHVPG,
(SEQ ID NO: 06)
HVPGGG,
(SEQ ID NO: 07)
HVPGG,
(SEQ ID NO: 08)
HKPGGG,
(SEQ ID NO: 09)
HKPGG,
(SEQ ID NO: 10)
KHVPGGG,
(SEQ ID NO: 11)
KHVPGG,
(SEQ ID NO: 12)
HQPGGG,
(SEQ ID NO: 13)
HQPGG,
(SEQ ID NO: 14)
VQIINK,
(SEQ ID NO: 15)
VQIINKK,
(SEQ ID NO: 16)
VQIINKKL,
(SEQ ID NO: 17)
QIINK,
(SEQ ID NO: 18)
QIINKK,
(SEQ ID NO: 19)
QIINKKL,
(SEQ ID NO: 25)
EIVYKSP,
(SEQ ID NO: 26)
IVYKSPV,
(SEQ ID NO: 27)
IVYK,
(SEQ ID NO: 325)
QIVYKS
(SEQ ID NO: 128)
EIVYKS
(SEQ ID NO: 411)
EIVYKP
(SEQ ID NO: 454)
GYTMHQD,
(SEQ ID NO: 456)
QGGYTMHQD,
(SEQ ID NO: 589)
VKSKIGSTE,
(SEQ ID NO: 590)
KSKIGSTE,
(SEQ ID NO: 598)
SKIGSTEN,
(SEQ ID NO: 605)
KIGSTENL,
(SEQ ID NO: 611)
IGSTENLK,
(SEQ ID NO: 616)
GSTENLKH,
(SEQ ID NO: 620)
STENLKHQ,
(SEQ ID NO: 476)
TENLKHQP,
(SEQ ID NO: 470)
ENLKHQPG,
(SEQ ID NO: 582)
VKSKIGST,
(SEQ ID NO: 755)
PDLKNVKS,
(SEQ ID NO: 756)
DLKNVKSK,
(SEQ ID NO: 757)
LKNVKSKI,
(SEQ ID NO: 758)
KNVKSKIG,
(SEQ ID NO: 759)
NVKSKIGS,
(SEQ ID NO: 465)
NLKHQPGG,
(SEQ ID NO: 460)
LKHQPGGG,
(SEQ ID NO: 760)
LDLSNVQS,
(SEQ ID NO: 761)
DLSNVQSK,
(SEQ ID NO: 762)
LSNVQSKC,
(SEQ ID NO: 763)
SNVQSKCG,
(SEQ ID NO: 764)
NVQSKCGS,
(SEQ ID NO: 626)
VQSKCGSK,
(SEQ ID NO: 634)
QSKCGSKD,
(SEQ ID NO: 642)
SKCGSKDN,
(SEQ ID NO: 649)
KCGSKDNI,
(SEQ ID NO: 258)
CGSKDNIK,
(SEQ ID NO: 653)
GSKDNIKH,
(SEQ ID NO: 271)
SKDNIKHV,
(SEQ ID NO: 278)
KDNIKHVP,
(SEQ ID NO: 285)
DNIKHVPG,
(SEQ ID NO: 292)
NIKHVPGG,
(SEQ ID NO: 297)
IKHVPGGG,
(SEQ ID NO: 765)
VDLSKVTS,
(SEQ ID NO: 766)
DLSKVTSK,
(SEQ ID NO: 767)
LSKVTSKC,
(SEQ ID NO: 768)
SKVTSKCG,
(SEQ ID NO: 769)
KVISKCGS,
(SEQ ID NO: 770)
VTSKCGSL,
(SEQ ID NO: 666)
TSKCGSLG,
(SEQ ID NO: 674)
SKCGSLGN,
(SEQ ID NO: 681)
KCGSLGNI,
(SEQ ID NO: 687)
CGSLGNIH,
(SEQ ID NO: 692)
GSLGNIHH,
(SEQ ID NO: 696)
SLGNIHHK,
(SEQ ID NO: 524)
LGNIHHKP,
(SEQ ID NO: 518)
GNIHHKPG,
(SEQ ID NO: 513)
NIHHKPGG,
(SEQ ID NO: 508)
IHHKPGGG,
(SEQ ID NO: 771)
LDFKDRVQ,
(SEQ ID NO: 772)
DFKDRVQS,
(SEQ ID NO: 773)
FKDRVQSK,
(SEQ ID NO: 774)
KDRVQSKI,
(SEQ ID NO: 775)
DRVQSKIG,
(SEQ ID NO: 776)
RVQSKIGS,
(SEQ ID NO: 702)
VQSKIGSL,
(SEQ ID NO: 709)
QSKIGSLD,
(SEQ ID NO: 717)
SKIGSLDN,
(SEQ ID NO: 724)
KIGSLDNI,
(SEQ ID NO: 729)
IGSLDNIT,
(SEQ ID NO: 734)
GSLDNITH,
(SEQ ID NO: 738)
SLDNITHV,
(SEQ ID NO: 561)
LDNITHVP,
(SEQ ID NO: 555)
DNITHVPG,
(SEQ ID NO: 551)
NITHVPGG,
and
(SEQ ID NO: 547)
ITHVPGGG.

In some embodiments, the immunogen comprises a tau peptide comprising, consisting of, or consisting essentially of, an amino acid sequence selected from the group consisting of any one of SEQ ID NO:20 to SEQ ID NO:24, SEQ ID NO:312 to SEQ ID NO:457. Each tau sequence optionally further comprising a C-terminal cysteine. In some embodiments, the immunogenic peptide comprises and amino acid sequence selected from the group consisting of

(SEQ ID NO: 20)
QIVYKSV,
(SEQ ID NO: 21)
EIVYKSV,
(SEQ ID NO: 22)
EIVYKPV,
(SEQ ID NO: 23)
CNIKHVP,
(SEQ ID NO: 24)
CNIKHVPG,
(SEQ ID NO: 449)
EAAGHVTQC,
(SEQ ID NO: 450)
EAAGHVTQAR,
(SEQ ID NO: 451)
AAGHVTQAC,
(SEQ ID NO: 452)
AGHVTQARC,
(SEQ ID NO: 453)
AGHVTQAR,
(SEQ ID NO: 455)
QGGYTMHC,
and
(SEQ ID NO: 457)
GGYTMHQC.

In some embodiments, the immunogenic peptide comprises, consists of, or consists essentially of, an amino acid sequence from the MTBR1 region (SEQ ID NO:751) of the long form of tau (SEQ ID NO:01), each with a C-terminal cysteine, -GGC, a C-terminal -GGGC, a N-terminal cysteine, CGG- or a N-terminal CGGG-. Examples include SEQ ID NO:777 to SEQ ID NO:785, SEQ ID NO:786 to SEQ ID NO:793, SEQ ID NO:843 to SEQ ID NO:850, and SEQ ID NO:851 to SEQ ID NO:859. In some embodiments, the peptide includes:

(SEQ ID NO: 777)
VKSKIGSTEGGC,
(SEQ ID NO: 778)
KSKIGSTEGGC,
(SEQ ID NO: 779)
SKIGSTENGGC,
(SEQ ID NO: 780)
KIGSTENLGGC,
(SEQ ID NO: 781)
IGSTENLKGGC,
(SEQ ID NO: 782)
GSTENLKHGGC,
(SEQ ID NO: 783)
STENLKHQGGC,
(SEQ ID NO: 784)
TENLKHQPGGC,
and
(SEQ ID NO: 785)
ENLKHQPGGGC.

In some embodiments, the immunogenic peptide comprises, consists of, or consists essentially of, an amino acid sequence from the MTBR2 region (SEQ ID NO:752) of the long form of tau (SEQ ID NO:01), each with a C-terminal cysteine, -GGC, a C-terminal -GGGC, a N-terminal cysteine, CGG- or a N-terminal CGGG-. Examples include SEQ ID NO:794 to SEQ ID NO:809 and SEQ ID NO:860 to SEQ ID NO:875. In some embodiments, the peptide includes:

(SEQ ID NO: 799)
VQSKCGSKGGC,
(SEQ ID NO: 800)
QSKCGSKDGGC,
(SEQ ID NO: 801)
SKCGSKDNGGC,
(SEQ ID NO: 802)
KCGSKDNIGGC,
(SEQ ID NO: 803)
CGSKDNIKGGC,
(SEQ ID NO: 804)
GSKDNIKHGGC,
(SEQ ID NO: 805)
SKDNIKHVGGC,
(SEQ ID NO: 806)
KDNIKHVPGGC,
and
(SEQ ID NO: 807)
DNIKHVPGGGC.

In some embodiments, the immunogenic peptide comprises, consists of, or consists essentially of, an amino acid sequence from the MTBR3 region (SEQ ID NO:753) of the long form of tau (SEQ ID NO:01), each with a C-terminal cysteine, -GGC, a C-terminal -GGGC, a N-terminal cysteine, CGG- or a N-terminal CGGG-. Examples include SEQ ID NO:810 to SEQ ID NO:825 and SEQ ID NO:876 to SEQ ID NO:891. In some embodiments, the peptide includes:

(SEQ ID NO: 815)
VTSKCGSLGGC,
(SEQ ID NO: 816)
TSKCGSLGGGC,
(SEQ ID NO: 817)
SKCGSLGNGGC,
(SEQ ID NO: 818)
KCGSLGNIGGC,
(SEQ ID NO: 819)
CGSLGNIHGGC,
(SEQ ID NO: 820)
GSLGNIHHGGC,
(SEQ ID NO: 821)
SLGNIHHKGGC,
(SEQ ID NO: 822)
LGNIHHKPGGC,
and
(SEQ ID NO: 823)
GNIHHKPGGGC.

In some embodiments, the immunogenic peptide comprises, consists of, or consists essentially of, an amino acid sequence from the MTBR4 region (SEQ ID NO:754) of the long form of tau (SEQ ID NO:01), each with a C-terminal cysteine, -GGC, a C-terminal -GGGC, a N-terminal cysteine, CGG- or a N-terminal CGGG-. Examples include SEQ ID NO:826 to SEQ ID NO:842 and SEQ ID NO:892 to SEQ ID NO:908. In some embodiments, the peptide includes:

(SEQ ID NO: 831)
RVQSKIGSGGC,
(SEQ ID NO: 832)
VQSKIGSLGGC,
(SEQ ID NO: 833)
QSKIGSLDGGC,
(SEQ ID NO: 834)
SKIGSLDNGGC,
(SEQ ID NO: 835)
KIGSLDNIGGC,
(SEQ ID NO: 836)
IGSLDNITGGC,
(SEQ ID NO: 837)
GSLDNITHGGC,
(SEQ ID NO: 838)
SLDNITHVGGC,
(SEQ ID NO: 839)
LDNITHVPGGC,
and
(SEQ ID NO: 840)
DNITHVPGGGC.

In some embodiments, the immunogenic peptide comprises, consists of, or consists essentially of, 5-13 amino acids from residues 244-400 of SEQ ID NO:01 or from residues 1-150 of SEQ ID NO:750, comprising at least one amino acid substitution. In one embodiment, the peptide comprises an amino acid sequence from within the Tau MTBR1 sequence (SEQ ID NO:751) that comprises SKIGSTENLKH (SEQ ID NO:909), and variants thereof. In some embodiments, the at least one amino acid substitutions comprises an isoleucine substitution for a lysine at position 10. In some embodiments, the at least one amino acid substitutions comprises a lysine or leucine substitution for a tyrosine at position 6, and in some embodiments, the at least one amino acid substitutions comprises an aspartic acid or glycine substitution for a glutamic acid at position 7.

In some embodiments, the peptide comprises an amino acid sequence selected from the group consisting of

(SEQ ID NO: 909)
SKIGSTENLKH,
(SEQ ID NO: 910)
SKIGSTENIKH,
(SEQ ID NO: 911)
SKIGSKDNLKH,
(SEQ ID NO: 912)
SKIGSKENIKH,
(SEQ ID NO: 913)
SKIGSLENLKH,
(SEQ ID NO: 914)
SKIGSLENIKH,
(SEQ ID NO: 915)
SKIGSTDNLKH,
(SEQ ID NO: 916)
SKIGSTDNIKH,
(SEQ ID NO: 917)
SKIGSKDNLKH,
(SEQ ID NO: 918)
SKIGSKDNIKH,
(SEQ ID NO: 919)
SKIGSLDNLKH,
(SEQ ID NO: 920)
SKIGSLDNIKH,
(SEQ ID NO: 921)
SKIGSTGNLKH,
(SEQ ID NO: 922)
SKIGSTGNIKH,
(SEQ ID NO: 923)
SKIGSKGNLKH,
(SEQ ID NO: 924)
SKIGSKGNIKH,
(SEQ ID NO: 925)
SKIGSLGNLKH,
(SEQ ID NO: 926)
SKIGSLGNIKH,

In some embodiments, the peptide comprises, consists essentially of, or consists of an amino acid sequence selected from the group consisting of SKIGSTDNIKH (SEQ ID NO:916), SKIGSKDNIKH (SEQ ID NO:918), or SKIGSLDNIKH (SEQ ID NO:920).

In some embodiments, the peptides further comprises, consists essentially of, or consists of, a C-terminal cysteine (—C), -GGC or -GGGC or an N-terminal cysteine (C—), CGG- or CGGG-.

In some embodiments, the peptide comprises an amino acid sequence selected from the group consisting of

(SEQ ID NO: 927)
SKIGSTENLKHGGC,
(SEQ ID NO: 928)
SKIGSTENIKHGGC,
(SEQ ID NO: 929)
SKIGSKDNLKHGGC,
(SEQ ID NO: 930)
SKIGSKENIKHGGC,
(SEQ ID NO: 931)
SKIGSLENLKHGGC,
(SEQ ID NO: 932)
SKIGSLENIKHGGC,
(SEQ ID NO: 933)
SKIGSTDNLKHGGC,
(SEQ ID NO: 934)
SKIGSTDNIKHGGC,
(SEQ ID NO: 935)
SKIGSKDNLKHGGC,
(SEQ ID NO: 936)
SKIGSKDNIKHGGC,
(SEQ ID NO: 937)
SKIGSLDNLKHGGC,
(SEQ ID NO: 938)
SKIGSLDNIKHGGC,
(SEQ ID NO: 939)
SKIGSTGNLKHGGC,
(SEQ ID NO: 940)
SKIGSTGNIKHGGC,
(SEQ ID NO: 941)
SKIGSKGNLKHGGC,
(SEQ ID NO: 942)
SKIGSKGNIKHGGC,
(SEQ ID NO: 943)
SKIGSLGNLKHGGC,
(SEQ ID NO: 944)
SKIGSLGNIKHGGC,
(SEQ ID NO: 945)
SKIGSTENLKHGGGC,
(SEQ ID NO: 946)
SKIGSTENIKHGGGC,
(SEQ ID NO: 947)
SKIGSKDNLKHGGGC,
(SEQ ID NO: 948)
SKIGSKENIKHGGGC,
(SEQ ID NO: 949)
SKIGSLENLKHGGGC,
(SEQ ID NO: 950)
SKIGSLENIKHGGGC,
(SEQ ID NO: 951)
SKIGSTDNLKHGGGC,
(SEQ ID NO: 952)
SKIGSTDNIKHGGGC,
(SEQ ID NO: 953)
SKIGSKDNLKHGGGC,
(SEQ ID NO: 954)
SKIGSKDNIKHGGGC,
(SEQ ID NO: 955)
SKIGSLDNLKHGGGC,
(SEQ ID NO: 956)
SKIGSLDNIKHGGGC,
(SEQ ID NO: 957)
SKIGSTGNLKHGGGC,
(SEQ ID NO: 958)
SKIGSTGNIKHGGGC,
(SEQ ID NO: 959)
SKIGSKGNLKHGGGC,
(SEQ ID NO: 960)
SKIGSKGNIKHGGGC,
(SEQ ID NO: 961)
SKIGSLGNLKHGGGC,
(SEQ ID NO: 962)
SKIGSLGNIKHGGGC,
(SEQ ID NO: 963)
CGGSKIGSTDNIKH,
(SEQ ID NO: 964)
CGGSKIGSKDNIKH,
(SEQ ID NO: 965)
CGGSKIGSLDNIKH,
(SEQ ID NO: 966)
CGGGSKIGSTDNIKH,
(SEQ ID NO: 967)
CGGGSKIGSKDNIKH,
and
(SEQ ID NO: 968)
CGGGSKIGSLDNIKH.

In some embodiments, the peptide or linker to the carrier, if present, further comprises a C-terminal cysteine (C). In some embodiments, the peptide further comprises a blocked amine at the N-terminus.

In some embodiments, the immunogen as described herein further comprises a linker to a carrier at a C-terminal portion of the polypeptide. In some embodiments, the immunogen as described herein further comprises a linker to a carrier at a N-terminal portion of the polypeptide. In some embodiments, where the C-terminal residues in the immunogen are either IVYKPV, VYKPV, YKPV, KPV, or PV, the linker is an amino acid linker that does not have a N-terminal glycine (e.g., GG, GAGA (SEQ ID NO:744)).

In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids or one (1) amino acid. In some embodiments, the linker is one amino acid, two amino acids, three amino acids, four amino acids, five amino acids, six amino acids, seven amino acids, eight amino acids, nine amino acids, or ten amino acids.

In some embodiments, the amino acid composition of a linker can mimic the composition of linkers found in natural multidomain proteins, where certain amino acids are overrepresented, underrepresented or equi-represented in natural linkers as compared to their abundance in whole protein. For example, threonine (Thr), serine (Ser), proline (Pro), glycine (Gly), aspartic acid (Asp), lysine (Lys), glutamine (Gln), asparagine (Asn), arginine (Arg), phenylalanine (Phe), glutamic acid (Glu) and alanine (Ala) are overrepresented in natural linkers. In contrast, isoleucine (Ile), tyrosine (Tyr), tryptophan (Trp), and cysteine (Cys) are underrepresented. In general, overrepresented amino acids were polar uncharged or charged residues, which constitute approximately 50% of naturally encoded amino acids, and Pro, Thr, and Gln were the most preferable amino acids for natural linkers. See, e.g., Chen, X. et al., “Fusion Protein Linkers: Property, Design and Functionality” Adv Drug Deliv Rev., 15; 65(10): 1357-1369 (2013).

In some embodiments, the amino acid composition of a linker can mimic the composition of linkers commonly found in recombinant proteins, which can generally by classified as flexible or rigid linkers. For example, flexible linkers found in recombinant proteins are generally composed of small, non-polar (e.g. Gly) or polar (e.g. Ser or Thr) amino acids whose small size provides flexibility and allows for mobility of the connecting functional domains. The incorporation of, e.g., Ser or Thr can maintain the stability of the linker in aqueous solutions by forming hydrogen bonds with the water molecules, and therefore can reduce interactions between the linker and the immunogens. In some embodiments, a linker comprises stretches of Gly and Ser residues (“GS” linker). An example of a widely used flexible linker is (Gly-Gly-Ser)n, (Gly-Gly-Gly-Ser)n (SEQ ID NO:969), or (Gly-Gly-Gly-Gly-Ser)n (SEQ ID NO:970), where n=1-3. Adjusting the copy number “n” can optimize a linker to achieve sufficient separation of the functional immunogen domains to, e.g., maximize an immunogenic response. Many other flexible linkers have been designed for recombinant fusion proteins that can be used herein. In some embodiments, linkers can be rich in small or polar amino acids such as Gly and Ser but also contain additional amino acids such as Thr and Ala to maintain flexibility, as well as polar amino acids such as Lys and Glu to improve solubility. See, e.g., Chen, X. et al., Adv Drug Deliv Rev., 15; 65(10): 1357-1369 (2013).

In some embodiments, a linker to a carrier may be included at the N-terminus of the peptide or polypeptide immunogen.

In some embodiments, the linker comprises an amino acid sequence of one of AA, AAA, KK, KKK, SS, SSS AGAG, GG, GGG, GAGA, KGKG, (GGS)n, (GGGS)n (SEQ ID NO:969), and (GGGGS)n (SEQ ID NO:970), where n=1-3. In some embodiments, the peptide further comprises a N- or C-terminal cysteine (regardless whether the peptide has a N- or C-terminal cysteine in the sequence identification number, e.g., SEQ ID NO:778 (KSKIGSTEGGC) can have a further C-terminal cysteine to yield KSKIGSTEGGC-C), and some embodiments that comprise a C- or N-terminal linker further comprise a C- or N-terminal cysteine on the C- or N-terminal end of the linker. In some embodiments, the immunogen peptides further comprise a blocked amine at the N-terminus.

In some embodiments, the two or more tau peptides are linked to form a tau polypeptide. The one or more tau peptides can be linked by an intra-peptide linker, which linker is as described above and herein. For example, a polypeptide linker located between the C-terminal of the first peptide and the N terminal of the second peptide. With or without the intra-peptide linker, the tau polypeptide may be arranged in any order. For example, a specific tau peptide (“tau A”) may be positioned at the N-terminal portion of a dual tau polypeptide and the same or a different tau peptide (for this example, a different tau, “tau B”) may be positioned at the C-terminal portion of the dual polypeptide. Or, the tau peptides in this example could be arranged in the opposite orientation (tau B N-terminal to tau A). Reference to a first peptide or a second peptide herein is not intended to suggest an order of the tau peptides in embodiments that comprise more than one tau peptide of the immunogens.

In addition, the C-terminal portion of the tau peptide or tau polypeptide can include a linker for conjugating the peptides or the polypeptide to a carrier, which linker is as described above and herein. In some embodiments, the tau peptide or polypeptide that comprise a linker further comprise a C-terminal cysteine on the C-terminal end of the linker. In some embodiments, the immunogen peptides further comprise a blocked amine at the N-terminus. In some embodiments, any of the tau peptides or polypeptides may include a C-terminal cysteine or a N-terminal cysteine without a linker.

When the tau peptides are linked to form a tau polypeptide, the linker may be a cleavable linker. As used herein, the term “cleavable linker” refers to any linker between the antigenic peptides that promotes or otherwise renders the tau polypeptide more susceptible to separation from each other by cleavage (for example, by endopeptidases, proteases, low pH or any other means that may occur within or around the antigen-presenting cell) and, thereby, processing by the antigen-presenting cell, than equivalent peptides lacking such a cleavable linker. In some embodiments, the cleavable linker is a protease-sensitive dipeptide or oligopeptide cleavable linker. In certain embodiments, the cleavable linker is sensitive to cleavage by a protease of the trypsin family of proteases. In some embodiments, where the C-terminal residues in the immunogen are either IVYKPV (SEQ ID NO:61), VYKPV (SEQ ID NO:62), YKPV (SEQ ID NO:63), KPV, or PV the cleavable linker is an amino acid linker that does not have a N-terminal glycine (e.g., GAGA). In some embodiments, the cleavable linker comprises an amino acid sequence including arginine-arginine (Arg-Arg), arginine-arginine-valine-arginine (Arg-Val-Arg-Arg; SEQ ID NO:743), Gly-Ala-Gly-Ala (SEQ ID NO:744), Ala-Gly-Ala-Gly (SEQ ID NO:745), Lys-Gly-Lys-Gly (SEQ ID NO:746), valine-citrulline (Val-Cit), valine-arginine (Val-Arg), valine-lysine (Val-Lys), valine-alanine (Val-Ala), and phenylalanine-lysine (Phe-Lys). In some embodiments, the cleavable linker is arginine-arginine (Arg-Arg).

In some embodiments of the disclosure, the tau polypeptide comprises an amino acid sequence selected from QIVYKPV (SEQ ID NO:02), or NIKHVP (SEQ ID NO:04), or NIKHVPG (SEQ ID NO:05), or EIVYKSV (SEQ ID NO:21), wherein XX is optionally appended to the C-terminal end of SEQ ID NOS:02, 04, 05, or 21, and a cysteine is optionally appended to the C-terminal end of SEQ ID NOS:02, 04, 05 or 21, or if XX is present, to the C-terminal end of the XX. XX can be AA, KK, SS, AGAG (SEQ ID NO:745), and KGKG (SEQ ID NO:746), and in some embodiments GG or GAGA (SEQ ID NO:744).

In some embodiments, the dual tau polypeptide is as follows:

[first peptide]-[linker 1]-[second peptide]-[linker 2]-[Cys],

wherein, the first peptide is a tau peptide and the second peptide is the same or different tau peptide, each of linker 1, linker 2 and [Cys] is optional, and linker 1 and linker 2 may be the same or different.

Examples of the tau peptide include any one of SEQ ID NO:02 to SEQ ID NO:742, SEQ ID NO:747 to SEQ ID NO:749, and SEQ ID NO:755 to SEQ ID NO:968.

[Linker 1] is optional, and when present, may be a linker or a cleavable linker, both as described above and herein. [Linker 2] is optional, and, when present, comprises a linker as described above and herein. Cys is optional and can be used to conjugate the polypeptide to a carrier.

In some embodiments, the dual tau polypeptide is as follows:

[Cys]-[linker 1]-[first peptide]-[linker 2]-[second peptide],

wherein, the first peptide is a tau peptide and the second peptide is the same or different tau peptide, each of linker 1, linker 2 and [Cys] is optional, and linker 1 and linker 2 may be the same or different.

[Linker 1] is optional, and when present, may be a linker or a cleavable linker, both as described above and herein. [Linker 2] is optional, and, when present, comprises a linker as described above and herein. Cys is optional and can be used to conjugate the polypeptide to a carrier.

Peptide-Carrier Immunogens

Tau peptides (and polypeptides thereof) are immunogens in accordance with the disclosure. In some embodiments, the peptides described herein can be linked to a suitable carrier to help elicit an immune response. Accordingly, one or more the peptides of the disclosure can be linked to a carrier. For example, the tau peptide may be linked to the carrier with or without a linker as described above and herein and, optionally, a C-terminal cysteine at C-terminal end of the linker or N-terminal cysteine at the N-terminal end of the linker and, if a linker is absent, at the C-terminal end or N-terminal end of the peptide, respectively. For example, each tau peptide may be linked to the carrier with or without spacer amino acids (e.g., Gly-Gly, Ala-Ala, Lys-Lys, Ser-Ser, Gly-Ala-Gly-Ala, Ala-Gly-Ala-Gly, or Lys-Gly-Lys-Gly and, optionally, a C-terminal or N-terminal cysteine to provide a linker between the peptide(s) and the carrier.

Suitable carriers include, but are not limited to serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking peptide immunogens of the invention to an immunostimulatory polymer molecule (e.g., tripalmitoyl-S-glycerine cysteine (Pam3Cys), mannan (a mannose polymer), or glucan (a β1-2 polymer)), cytokines (e.g., IL-1, IL-1 alpha and β peptides, IL-2, γ-INF, IL-10, GM-CSF), and chemokines (e.g., MIP1-α and β, and RANTES). Additional carriers include virus-like particles. In some compositions, immunogenic peptides can also be linked to carriers by chemical crosslinking. Techniques for linking an immunogen to a carrier include the formation of disulfide linkages using N-succinimidyl-3-(2-pyridyl-thio)propionate (SPDP), and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine resides on one protein and an amide linkage through the epsilon-amino on a lysine, or other free amino group in other amino acids. In some embodiments, chemical crosslinking can comprise use of SBAP (succinimidyl 3-(bromoacetamido)propionate), which is a short (6.2 angstrom) cross-linker for amine-to-sulfhydryl conjugation via N-hydroxysuccinimide (NHS) ester and bromoacetyl reactive groups. A variety of such disulfide/amide-forming agents are described by Jansen et al., “Immunotoxins: Hybrid Molecules Combining High Specificity and Potent Cytotoxicity” Immunological Reviews 62:185-216 (February 1982). Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimido-methyl)cy-clohexane-1-carboxylic acid. The carboxyl groups can be activated by combining them with succinimide or 1-hydroxyl-2-nitro-4-sulfonic acid, sodium salt. Virus-like particles (VLPs), also called pseudovirions or virus-derived particles, represent subunit structures composed of multiple copies of a viral capsid and/or envelope protein capable of self-assembly into VLPs of defined spherical symmetry in vivo. (Powilleit, et al., (2007) PLoS ONE 2(5):e415.) Alternatively, peptide immunogens can be linked to at least one artificial T-cell epitope capable of binding a large proportion of MHC Class II molecules, such as the pan DR epitope (“PADRE”). Pan DR-binding peptides (PADRE) are described in U.S. Pat. No. 5,736,142, WO 95/07707, and Alexander J et al. Immunity, 1:751-761 (1994).

Active immunogens can be presented in multimeric form in which multiple copies of an immunogen are presented on a carrier as a single covalent molecule. In some embodiments, the carrier includes various forms of the tau peptide. For instance, the tau peptide of the immunogen can include peptides that have different tau antigens in different orders, or may be present with or without an intrapeptide linker and/or a linker to a carrier.

In some compositions, the immunogenic peptides can also be expressed as fusion proteins with carriers. In certain compositions, the immunogenic peptides can be linked at the amino terminus, the carboxyl terminus, or internally to the carrier. In some compositions, the carrier is CRM197. In some compositions, the carrier is diphtheria toxoid.

Nucleic Acids

The disclosure further provides nucleic acids encoding any of the tau peptides as disclosed herein. The nucleic acid immunotherapy compositions as disclosed herein, comprise, consist of or consisting essentially of a nucleic acid sequence encoding one or more tau peptides as disclosed herein. For example, the tau peptide can comprise a sequence of 3-13 (e.g., 7-13, 5-10, 7-11, 8) amino acids in length and from residues 244-400 of SEQ ID NO:01 or 1-150 of SEQ ID NO:750. Accordingly, and as a non-limiting example, one or more nucleic acids encoding any of SEQ ID NO:02 to SEQ ID NO:742, SEQ ID NO:747 to SEQ ID NO:749, or SEQ ID NO:755 to SEQ ID NO:968 provide an immunogen and pharmaceutical composition of the disclosure. In certain embodiments, the peptide sequences may be encoded by the same or separate nucleic acid sequences. In some embodiments, the nucleic acid sequences may also encode a linker to a carrier and/or a N- or C-terminal cysteine as described herein. In addition, when a single nucleic acid sequence encodes more than one tau peptide, the sequence may also encode a linker as described herein. The nucleic acid compositions described herein (pharmaceutical compositions) can be used in methods for treating or effecting prophylaxis and/or prevention of Alzheimer's disease. In another embodiment, the nucleic acid immunotherapy compositions as disclosed herein provide compositions for reducing brain tau.

A nucleic acid such as DNA that encodes an immunogen and is used as a vaccine can be referred to as a “DNA immunogen” or “DNA vaccine” as the encoded polypeptides ae expressed in vivo after administration of the DNA. DNA vaccines are intended to induce antibodies against the proteins of interest they encode in a subject by: integrating DNA encoding the proteins of interest into a vector (a plasmid or virus); administering the vector to the subject; and expressing the proteins of interest in the subject in which the vector has been administered to stimulate the immune system of the subject. A DNA vaccine remains in the body of the subject long after its administration and continues to slowly produce the encoded proteins. Thus, excessive immune responses can be avoided. DNA vaccines can also be modified using genetic engineering techniques. Optionally, such nucleic acids further encode a signal peptide and can be expressed with the signal peptide linked to peptide. Coding sequences of nucleic acids can be operably linked with regulatory sequences to ensure expression of the coding sequences, such as a promoter, enhancer, ribosome binding site, transcription termination signal, and the like. The nucleic acids encoding tau can occur in isolated form or can be cloned into one or more vectors. The nucleic acids can be synthesized by, for example, solid state synthesis or PCR of overlapping oligonucleotides. Nucleic acids encoding tau peptide and tau polypeptides with and without linkers and/or cleavable linkers and with or without protein-based carriers can be joined as one contiguous nucleic acid, e.g., within an expression vector.

DNA is more stable than RNA, but DNA involves some potential safety risks such as induction of anti-DNA antibodies, thus in some embodiments, the nucleic acid can be RNA. RNA nucleic acid that encodes an immunogen and is used as a vaccine can be referred to as a “RNA immunogen” or “RNA vaccine” or “mRNA vaccine” as the encoded polypeptides are expressed in vivo after administration of the RNA. Ribonucleic acid (RNA) vaccines can safely direct a subject's cellular machinery to produce one or more a polypeptide(s) of interest. In some embodiments, a RNA vaccine can be a non-replicating mRNA (messenger-RNA) or a virally derived, self-amplifying RNA. mRNA-based vaccines encode the antigens of interest and contain 5′ and 3′ untranslated regions (UTRs), whereas self-amplifying RNAs encode not only the antigens, but also the viral replication machinery that enables intracellular RNA amplification and abundant protein expression. In vitro transcribed mRNA can be produced from a linear DNA template using a T7, a T3 or an Sp6 phage RNA polymerase. The resulting product can contain an open reading frame that encodes the peptides of interest as disclosed herein, flanking 5′- and 3-UTR sequences, a 5′ cap and a poly(A) tail. In some embodiments, a RNA vaccine can comprise trans-amplifying RNA (for example, see Beissert el al., Molecular Therapy January 2020 28(1):119-128). In certain embodiments, RNA vaccines encode a tau peptide as disclosed herein, and are capable of expressing the tau peptides, in particular if transferred into a cell such as an immature antigen presenting cell. RNA may also contain sequences which encode other polypeptide sequences such as immune stimulating elements. In some embodiments, the RNA of a RNA vaccine can be modified RNA. The term “modified” in the context of the RNA can include any modification of RNA which is not naturally present in RNA. For example, modified RNA can refer to RNA with a 5′-cap; however, RNA may comprise further modifications. A 5′-cap can be modified to possess the ability to stabilize RNA when attached thereto. In certain embodiments, a further modification may be an extension or truncation of the naturally occurring poly(A) tail or an alteration of the 5′- or 3′-untranslated regions (UTR). In some embodiments, the RNA (e.g., mRNA) vaccine is formulated in an effective amount to produce an antigen specific immune response in a subject. For example, the RNA vaccine formulation is administered to a subject in order to stimulate the humoral and/or cellular immune system of the subject against the tau antigens, and thus may further comprise one or more adjuvant(s), diluents, carriers, and/or excipients, and is applied to the subject in any suitable route in order to elicit a protective and/or therapeutic immune reaction against the tau antigens.

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology. Vol. 2, Wiley-Interscience, New York, (current edition); Kriegler, Gene Transfer and Expression: A Laboratory Manual (1990); Glover, D M, ed, DNA Cloning: A Practical Approach, vol. 1 & 11, IRL Press, 1985; Albers, B. et al., Molecular Biology of the Cell, 2^{nd} Ed., Garland Publishing, Inc., New York, N.Y. (1989); Watson, J D et al., Recombinant DNA, 2′ Ed., Scientific American Books, New York, 1992; and Old, R W et al., Principles of Gene Manipulation: An Introduction to Genetic Engineering, 2^{nd} Ed., University of California Press, Berkeley, Calif. (1981).

Techniques for the manipulation of nucleic acids, such as, e.g., generating mutations in sequences, sub-cloning, labeling probes, sequencing, hybridization and the like are well described in the scientific and patent literature. See, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immunofluorescence assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

Pharmaceutical Compositions

Each of the peptides and immunogens described herein can be presented in a pharmaceutical composition that is often administered with pharmaceutically acceptable adjuvants and pharmaceutically acceptable excipients. The adjuvant increases the titer of induced antibodies and/or the binding affinity of induced antibodies relative to the situation if the peptide were used alone. A variety of adjuvants can be used in combination with an immunogen of the disclosure to elicit an immune response. Some adjuvants augment the intrinsic response to an immunogen without causing conformational changes in the immunogen that affect the qualitative form of the response. An adjuvant may be a natural compound, a modified version of or derivative of a natural compound, or a synthetic compound.

Some adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). As used herein, MPL refers to natural and synthetic versions of MPL. Examples of synthetic versions include PHAD®, 3D-PHAD® and 3D(6A)-PHAD® (Avanti Polar Lipids (Croda), Alabaster, Alabama).

QS-21 is a triterpene glycoside or saponin isolated from the bark of the Quillaja Saponaria molina tree found in South America (see Kensil et al., in Vaccine Design: The Subunit and Adjuvant Approach (eds. Powell & Newman, Plenum Press, N Y, 1995)) QS-21 products include Stimulon® (Antigenics, Inc., New York, NY; now Agenus, Inc. Lexington, MA) and QS-21 Vaccine Adjuvant (Desert King, San Diego, CA). QS-21 has been disclosed, characterized, and evaluated in U.S. Pat. Nos. 5,057,540, and 8,034,348 the disclosures of which are herein incorporated by reference. Additionally. QS-21 has been evaluated in numerous clinical trials in various dosages. See, NCT00960531 (clinicaltrials.gov/ct2/show/study/NCT00960531), Hall et al., Curr Alzheimer Res. 2017 July; 14(7): 696-708 (evaluated 50 mcg of QS-21 in with various doses of vaccine ACC-001); Gilman S, et al., “Clinical effects of Abeta immunization (AN1792) in patients with AD in an interrupted trial”, AN1792(QS-21)-201 Study Team. Neurology. 2005 May 10; 64(9):1553-62; Wald A, el al., “Safety and immunogenicity of long HSV-2 peptides complexed with rhHsc70 in HSV-2 seropositive persons” Vaccine 2011. Nov. 3; 29(47):8520-8529; and Cunningham et al., Efficacy of the Herpes Zoster Subunit Vaccine in Adults 70 Years of Age or Older. NEJM. 2016 Sep. 15; 375(11):1019-32. Vaccine 2011. Nov. 3; 29(47):8520-8529. QS-21 is used in FDA approved vaccines including SHINGRIX. SHINGRIX contains 50 mcg of QS-21. In certain embodiments, the amount of QS-21 is from about 10 μg to about 500 μg.

TQL1055 is an analogue of QS-21 (Adjuvance Technologies, Lincoln, NE). The semi-synthetic TQL1055 has been characterized in comparison to QS-21 as having high purity, increased stability, decreased local tolerability, decreased systemic tolerability. TQL1055 has been disclosed, characterized, and evaluated in US20180327436A1, WO2018191598A1, WO2018200656A, and WO2019079160A1, the disclosures of which are herein incorporated by reference. US20180327436A1 teaches that 2.5 fold more TQ1055 was superior to 20 μg QS-21 but there was not an improvement over 50 μg TQ1055. However, unlike QS-21 there was no increase in either weight loss or hemolysis of RBC as the TQL1055 dose increased. WO2018200656A1 teaches that with an optimal amount of TQ1055, one can lower the amount of antigen and achieve superior titers. In certain embodiments, the amount of TQL1055 is from about 10 μg to about 500 μg.

Other adjuvants are oil in water emulsions (such as squalene or peanut oil), optionally in combination with immune stimulants, such as monophosphoryl lipid A (see Stoute el al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween 80. Ribi also contains refined mycobacterial products which act as immunostimulants and bacterial monophosphoryl lipid A. Other adjuvants can be CpG oligonucleotides (see WO 98/40100), cytokines (e.g., IL-1, IL-1 alpha and β peptides, IL-2, γ-INF, IL-10, GM-CSF), chemokines (e.g., MIP1-α and β, and RANTES), saponins, RNA, and/or TLR agonists (for example, TLR4 agonists such as MPL and synthetic MPL molecules), aminoalkyl glucosaminide phosphate and other TLR4 agonists. Adjuvants can be administered as a component of a therapeutic composition with an active agent or can be administered separately, before, concurrently with, or after administration of the therapeutic agent.

In various embodiments of the disclosure, the adjuvant is QS-21 (Stimulon™). In some compositions, the adjuvant is MPL. In certain embodiments, the amount of MPL is from about 10 μg to about 500 μg. In some compositions, the adjuvant is TQL1055. In certain embodiments, the amount of TQL1055 is from about 10 μg to about 500 μg. In some compositions, the adjuvant is QS21. In certain embodiments, the amount of QS21 is from about 10 μg to about 500 μg. In some compositions, the adjuvant is a combination of MPL and QS-21. In some compositions, the adjuvant is a combination of MPL and TQL1055. In some compositions, the adjuvant can be in a liposomal formulation.

In addition, some embodiments of the disclosure can comprise a multiple antigen presenting system (MAP). Multiple antigen-presenting peptide vaccine systems have been developed to avoid the adverse effects associated with conventional vaccines (i.e., live-attenuated, killed or inactivated pathogens), carrier proteins and cytotoxic adjuvants. Two main approaches have been used to develop multiple antigen presenting peptide vaccine systems: (1) the addition of functional components, e.g., T-cell epitopes, cell-penetrating peptides, and lipophilic moieties; and (2) synthetic approaches using size-defined nanomaterials, e.g., self-assembling peptides, non-peptidic dendrimers, and gold nanoparticles, as antigen-displaying platforms. Use of a multiple antigenic peptide (MAP) system can improve the sometimes poor immunogenicity of subunit peptide vaccines. In a MAP system, multiple copies of antigenic peptides are simultaneously bound to the α- and ε-amino groups of a non-immunogenic Lys-based dendritic scaffold, helping to confer stability from degradation, thus enhancing molecular recognition by immune cells, and induction of stronger immune responses compared with small antigenic peptides alone. In some compositions, the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the peptides of the disclosure can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, peptide compositions can be in lyophilized form for constitution with a suitable vehicle, e.g., sterile pyrogen-free water, before use.

Peptides (and optionally a carrier fused to the peptide(s)) can also be administered in the form of a nucleic acid encoding the peptide(s) and expressed in situ in a subject. A nucleic acid segment encoding an immunogen is typically linked to regulatory elements, such as a promoter and enhancer that allow expression of the DNA segment in the intended target cells of a subject. For expression in blood cells, as is desirable for induction of an immune response, promoter and enhancer elements from, for example, light or heavy chain immunoglobulin genes or the CMV major intermediate early promoter and enhancer are suitable to direct expression. The linked regulatory elements and coding sequences are often cloned into a vector.

DNA and RNA can be delivered in naked form (i.e., without colloidal or encapsulating materials). Alternatively, a number of viral vector systems can be used including retroviral systems (see, e.g., Boris-Lawrie and Temin, Cur. Opin. Genet. Develop. 3(1), 102-109 (1993)); adenoviral vectors (see, e.g., Bett et al, J. Virol. 67(10), 5911-21 (1993)); adeno-associated virus vectors (see, e.g., Zhou et al., J. Exp. Med. 179(6), 1867-75 (1994)), viral vectors from the pox family including vaccinia virus and the avian pox viruses, viral vectors from the alpha virus genus such as those derived from Sindbis and Semliki Forest Viruses (see, e.g., Dubensky el al., J. Virol. 70(1), 508-519 (1996)), Venezuelan equine encephalitis virus (see U.S. Pat. No. 5,643,576) and rhabdoviruses, such as vesicular stomatitis virus (see WO 96/34625) and papillomaviruses (WO 94/12629; Ohe et al., Human Gene Therapy 6(3), 325-333 (1995); and Xiao & Brandsma, Nucleic Acids. Res. 24(13):2620-2622 (1996)).

DNA and RNA encoding an immunogen, or a vector containing the same, can be packaged into liposomes, nanoparticles or lipoproteins complexes. Other suitable polymers include, for example, protamine liposomes, polysaccharide particles, cationic nanoemulsion, cationic polymer, cationic polymer liposome, cationic lipid nanoparticles, cationic lipid, cholesterol nanoparticles, cationic lipid-cholesterol, PEG nanoparticle, or dendrimer nanoparticles. Additional suitable lipids and related analogs are described by U.S. Pat. Nos. 5,208,036, 5,264,618, 5,279,833, and 5,283,185, each of which is herein incorporated by reference in its entirety. Vectors and DNA encoding an immunogen can also be adsorbed to or associated with particulate carriers, examples of which include polymethyl methacrylate polymers and polylactides and poly(lactide-co-glycolides), (see, e.g., McGee et al., J. Micro Encap, March-April 1997; 14(2):197-210).

Pharmaceutically acceptable carrier compositions can also include additives, including water, pharmaceutically acceptable organic solvents, collagen, polyvinyl alcohol, polyvinylpyrrolidone, carboxyvinyl polymers, carboxymethylcellulose sodium, sodium polyacrylate, sodium alginate, water-soluble dextran, carboxymethyl starch sodium, pectin, methylcellulose, ethylcellulose, xanthan gum, gum arabic, casein, agar, polyethylene glycol, diglycerine, glycerine, propylene glycol, petrolatum, paraffin, stearyl alcohol, stearic acid, human serum albumin, mannitol, sorbitol, lactose, and surfactants acceptable as pharmaceutical additives.

Subjects Amenable to Treatment

The presence of neurofibrillary tangles has been found in several diseases and tauopathies including Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, ganglioglioma and gangliocytoma, meningioangiomatosis, amyotrophic lateral sclerosis/parkinsonism dementia complex of Guam, subacute sclerosing panencephalitis, corticobasal degeneration (CBD), dementia with Lewy bodies, Lewy body variant of Alzheimer's disease (LBVAD), chronic traumatic encephalopathy (CTE), globular glial tauopathy (GGT), Parkinson's disease, progressive supranuclear palsy (PSP), dry age-related macular degeneration (AMD), and inclusion-body myositis.

The compositions and methods of the disclosure can be used in treatment or prophylaxis of any of these diseases. Because of the widespread association between neurological diseases and tau, the compositions and methods of the disclosure can be used in treatment or prophylaxis of any subject showing elevated levels of tau (e.g., in the CSF) compared with a mean value in individuals without neurological disease. The compositions and methods of the disclosure can also be used in treatment or prophylaxis of neurological disease in individuals having a mutation in tau associated with neurological disease. The methods are particularly suitable for treatment or prophylaxis of Alzheimer's disease.

Subjects amenable to treatment include individuals at risk of disease but not showing symptoms, as well as patients presently showing symptoms, including treatment naïve subjects that have not been previous treated for disease. Subjects at risk of disease include those in an aging population, asymptomatic subjects with tau pathologies and having a known genetic risk of disease. Such individuals include those having relatives who have experienced this disease, and those whose risk is determined by analysis of genetic or biochemical markers. Genetic markers of risk include mutations in tau, as well as mutations in other genes associated with neurological disease. For example, the ApoE4 allele in heterozygous and even more so in homozygous form is associated with risk of Alzheimer's disease (AD). Other markers of risk of Alzheimer's disease include mutations in the APP gene, particularly mutations at position 717 and positions 670 and 671 referred to as the Hardy and Swedish mutations respectively, mutations in the presenilin genes, PS1 and PS2, a family history of AD, hypercholesterolemia or atherosclerosis. Individuals presently suffering from Alzheimer's disease can be recognized by PET imaging, from characteristic dementia, as well as the presence of risk factors described above. In addition, a number of diagnostic tests are available for identifying individuals who have AD. These include measurement of CSF or blood tau or phospho-tau levels. Elevated tau or phospho-tau levels signify the presence of AD. Some mutations associated with Parkinson's disease, for example, Ala30Pro or Ala53Thr, or mutations in other genes associated with Parkinson's disease such as leucine-rich repeat kinase (LRRK2 or PARK8) appear to be associated with some AD. Subjects can also be diagnosed with any of the neurological diseases mentioned above by the criteria of the DSM IV TR.

In asymptomatic subjects, treatment can begin at any age (e.g., 10, 20, 30, or more). Usually, however, it is not necessary to begin treatment until a subject reaches 20, 30, 40, 50, 60, 70, 80 or 90 years of age. Treatment typically entails multiple dosages over a period of time. Treatment can be monitored by assaying antibody levels over time. If the response falls, a booster dosage is indicated. In the case of potential Down's syndrome patients, treatment can begin antenatally by administering therapeutic agent to the mother or shortly after birth.

Methods of Treatments and Uses

The disclosure provides methods of inhibiting or reducing aggregation of or tau in a subject having or at risk of developing Alzheimer's disease. The methods include administering to the subject the compositions as disclosed herein. A therapeutically effective amount is a dosage that, when given for an effective period of time, achieves the desired immunological or clinical effect. Dosage regimens may be adjusted to provide the optimum therapeutic response. For example, several divided doses may be administered at set intervals (e.g., weekly, monthly) or the dose may be proportionally reduced as indicated by the exigencies of the therapeutic situation.

In prophylactic applications, the compositions described herein can be administered to a subject susceptible to, or otherwise at risk of a disease (e.g., Alzheimer's disease) in a regimen (dose, frequency and route of administration) effective to reduce the risk, lessen the severity, or delay the onset of at least one sign or symptom of the disease. In particular, the regimen is effective to inhibit or delay tau or phospho-tau and paired filaments, tangles, and/or aggregates formed from them in the brain, and/or inhibit or delay its toxic effects and/or inhibit/or delay development of behavioral deficits. In therapeutic applications, the compositions described herein are administered to a subject suspected of, or a patient already suffering from a disease (e.g., Alzheimer's disease) in a regimen (dose, frequency and route of administration) effective to ameliorate or at least inhibit further deterioration of at least one sign or symptom of the disease. In particular, the regimen is preferably effective to reduce or at least inhibit further increase of levels of tau or phospho-tau and paired filaments, tangles, and/or aggregates formed from them, associated toxicities and/or behavioral deficits.

A regimen is considered therapeutically or prophylactically effective if an individual treated achieves an outcome more favorable than the mean outcome in a control population of comparable subjects not treated by methods of the invention, or if a more favorable outcome is demonstrated in treated subjects versus control subjects in a controlled clinical trial (e.g., a phase II, phase II/III or phase III trial) at the p<0.05 or 0.01 or even 0.001 level.

Effective doses of vary depending on many different factors, such as means of administration, target site, physiological state of the patient, whether the patient is an ApoE carrier, whether the patient is human or an animal, other medications administered, and whether treatment is prophylactic or therapeutic.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times. In some embodiments, a RNA (e.g., mRNA) vaccine is administered to a subject by intradermal, intramuscular injection, or by intranasal administration.

In some embodiments, the amount of an agent for active immunotherapy varies from 1 to 1,000 micrograms (μg), or from 0.1-500 μg, or from 10 to 500 μg, or from 50 to 250 μg per patient and can be from 1-100 or 1-10 μg per injection for human administration. The timing of injections can vary significantly from once a day, to once a week, to once a month, to once a year, to once a decade. A typical regimen consists of an immunization followed by booster injections at time intervals, such as 6 week intervals or two months. Another regimen consists of an immunization followed by one or more booster injections 1, 2, 3, 4, 5, 6, or 12 months later. Another regimen entails an injection every two months for life. Alternatively, booster injections can be on an irregular basis as indicated by monitoring of immune response. The frequency of administration may be once or more as long as the side effects are within a clinically acceptable range.

In some embodiments, the compositions or methods as disclosed herein comprise administering to a subject a nucleic acid vaccine comprising one or more DNA or RNA polynucleotides having an open reading frame encoding a peptide and, optionally, a second peptide, wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320400 μg, 40-380 μg, 40-100 μg, 100-400 μg, 200-400 μg, or 300-400 μg per dose. In some embodiments, the nucleic acid is administered to the subject by intradermal or intramuscular injection. In some embodiments, the nucleic acid is administered to the subject on day zero. In some embodiments, a second dose of the nucleic acid is administered to the subject on day seven, or fourteen, or twenty one.

The compositions described herein are preferably administered via a peripheral route (i.e., one in which the administered composition results in a robust immune response and/or the induced antibody population crosses the blood brain barrier to reach an intended site in the brain, spinal cord, or eye. For peripheral diseases, the induced antibodies leave the vasculature to reach the intended peripheral organs. Routes of administration include oral, subcutaneous, intranasal, intradermal, or intramuscular. Some routes for active immunization are subcutaneous and intramuscular. Intramuscular administration and subcutaneous administration can be made at a single site or multiple sites. Intramuscular injection is most typically performed in the arm or leg muscles. In some methods, agents are injected directly into a particular tissue where deposits have accumulated.

The number of dosages administered can be adjusted to result in a more robust immune response (for example, higher titers). For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation.

An effective amount of a DNA or RNA encoded immunogen can be between about 1 nanogram and about 1 gram per kilogram of body weight of the recipient, or about between about 0.1 μg/kg and about 10 mg/kg, or about between about 1 μg/kg and about 1 mg/kg. Dosage forms suitable for internal administration preferably contain (for the latter dose range) from about 0.1 μg to 100 μg of active ingredient per unit. The active ingredient may vary from 0.5 to 95% by weight based on the total weight of the composition. Alternatively, an effective dose of dendritic cells loaded with the antigen is between about 10⁴ and 10⁸ cells. Those skilled in the art of immunotherapy will be able to adjust these doses without undue experimentation.

The nucleic acid compositions may be administered in a convenient manner, e.g., injection by a convenient and effective route. Routes can include, but are not limited to, intradermal “gene gun” delivery or intramuscular injection. The modified dendritic cells are administered by subcutaneous, intravenous or intramuscular routes. Other possible routes include oral administration, intrathecal, inhalation, transdermal application, or rectal administration.

Depending on the route of administration, the composition may be coated in a material to protect the compound from the action of enzymes, acids and other natural conditions which may inactivate the compound. Thus, it may be necessary to coat the composition with, or co-administer the composition with, a material to prevent its inactivation. For example, an enzyme inhibitors of nucleases or proteases (e.g., pancreatic trypsin inhibitor, diisopropylluorophosphate and trasylol) or in an appropriate carrier such as liposomes (including water-in-oil-in-water emulsions as well as conventional liposomes (Strejan et al., J. Neuroimmunol 7(1):27-41, 1984).

The immunotherapeutic compositions disclosed herein may also be used in combination with other treatments for diseases associated with the accumulation of tau, for example, anti-tau antibodies such as antibodies that specifically bind to any of the tau epitopes disclosed herein, ABBV-8E12, gosuranemab, zagotenemab, RG-6100, BIIB076 or any of the antibodies disclosed in WO2014/165271, U.S. Pat. No. 10,501,531, WO2017/191559, WO2017/191560, WO2017/191561, US2019/0330314, US2019/0330316, and WO2018/204546. In some combination therapy methods, the patient receives passive immunotherapy prior to the active immunotherapy methods disclosed herein. In other methods, the patient receives passive and active immunotherapy during the same period of treatment. Alternatively, patients may receive active immunotherapy prior to passive immunotherapy. Combinations may also include small molecule therapies and non-immunogenic therapies such as RAZADYNE® (galantamine), EXELON® (rivastigmine), and ARICEPT® (donepezil) and other compositions that improve the function of nerve cells in the brain.

The compositions of the disclosure may be used in the manufacture of medicaments for the treatment regimens described herein.

Treatment Regimens

Desired outcomes of the methods of treatment as disclosed herein vary according to the disease and patient profile and are determinable to those skilled in the art. Desired outcomes include an improvement in the patient's health status. Generally, desired outcomes include measurable indices such as reduction or clearance of pathologic tau tangles and/or aggregates, we well as other associated pathologies such as amyloid fibrils, decreased or inhibited amyloid aggregation and/or deposition of amyloid fibrils, and increased immune response to pathologic species, e.g., tau-containing tangles and/or tau-containing aggregates. Desired outcomes also include amelioration of tau disease-specific symptoms. As used herein, relative terms such as “improve,” “increase,” or “reduce” indicate values relative to a control, such as a measurement in the same individual prior to initiation of treatment described herein, or a measurement in a control individual or group. A control individual is an individual afflicted with the same disease or tauopathy as the individual being treated, who is about the same age as the individual being treated (to ensure that the stages of the disease in the treated individual and the control individual are comparable), but who has not received treatment using the disclosed immunogens. Alternatively, a control individual is a healthy individual, who is about the same age as the individual being treated. Changes or improvements in response to therapy are generally statistically significant and described by a p-value less than or equal to 0.1, less than 0.05, less than 0.01, less than 0.005, or less than 0.001 may be regarded as significant.

Effective doses of the compositions as disclosed herein, for the treatment of a subject vary depending upon many different factors, including means of administration, target site, physiological state of the patient, whether the patient is human or an animal, other medications administered, if any, and whether treatment is prophylactic or therapeutic. Treatment dosages can be titrated to optimize safety and efficacy. The amount of immunogen can also depend on whether adjuvant is also administered, with higher dosages being required in the absence of adjuvant. The amount of an immunogen for administration sometimes varies from 1-500 μg per patient and more usually from 5-500 μg per injection for human administration. Occasionally, a higher dose of 1-2 mg per dosage is used. Typically, about 10, 20, 50 or 100 μg is used for each human dosage. The timing of dosages can vary significantly from once a day, to once a year, to once a decade. On any given day that a dosage of immunogen is given, the dosage is greater than 1 μg/patient and usually greater than 10 μg/patient if adjuvant is also administered, and greater than 10 μg/patient and usually greater than 100 μg/patient in the absence of adjuvant. A typical regimen consists of an immunization followed by booster dosage(s) at 6-week intervals. Another regimen consists of an immunization followed by booster dosage(s) 1, 2, 3, 4, 5, 6, or 12 months later. Another regimen entails dosage(s) every two months for life. Alternatively, booster dosage(s) can be on an irregular basis as indicated by monitoring of immune response.

When administered in combination with a second treatment for Alzheimer's disease, such as, Razadyne® (galantamine), Exelon® (rivastigmine), and Aricept® (donepezil), the second treatment can be administered according the product label or as necessary in view of the treatment with the compositions of the disclosure.

Kits

The disclosure further provides kits (e.g., containers) comprising the compositions disclosed herein and related materials, such as instructions for use (e.g., package insert). The instructions for use may contain, for example, instructions for administration of the compositions and optionally one or more additional agents. The containers of peptide and/or nucleic acid compositions may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products. Kits can also include a second container comprising a pharmaceutically acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

The following are provided for exemplification purposes only and are not intended to limit the scope of the invention described in broad terms above. All references cited in this disclosure are incorporated herein by reference.

Uses

Each of the peptides, polypeptides, immunogens, and pharmaceutical compositions described herein may be for use in treating one or more of the diseases as described herein. In addition, each of the peptides, polypeptides, immunogens, and pharmaceutical compositions described herein may be for use in methods for treating one or more of the diseases as described herein. Each of the peptides, polypeptides, immunogens, and pharmaceutical compositions described herein may be used in a method for manufacturing a medicament for treating or use in treating one or more of the diseases as described herein.

All U.S. and international patent applications identified herein are incorporated by reference in their entirety.

### EXAMPLES

### Example 1: Immunogens

Immunogens were selected for evaluation in vaccine peptide constructs. Some immunogens comprise a tau peptide comprising 3-10 amino acids from tau. Other immunogens comprise an engineered tau immunogen.

Engineered Tau Immunogens

Certain immunogenic peptides were designed and selected to (i) raise antibodies that bind within the within microtubule binding repeats (MTBRs) of human Tau protein, (ii) be less likely to generate an unwanted T cell-mediated autoimmune response, and (iii) be less likely to raise antibodies that would cross-react with other human proteins.

First, sequence analysis and 3D modeling of Tau MTBRs was conducted to identify amino acid residues that may be important for raising antibodies that bind MTBR. The results of these analyses were used to design synthetic Tau immunogenic peptides with conserved residues and shuffled interspersed residues. Resulting engineered synthetic peptides are listed in Table 1.

TABLE 1
Engineered Tau immunogenic peptides

| Engineered tau immunogenic | SEQ |
|---|---|
| peptides sequence | ID NO: |
| SKIGSTENLKH | 909 |
| SKIGSTENIKH | 910 |
| SKIGSKDNLKH | 911 |
| SKIGSKENIKH | 912 |
| SKIGSLENLKH | 913 |
| SKIGSLENIKH | 914 |
| SKIGSTDNLKH | 915 |
| SKIGSTDNIKH | 916 |
| SKIGSKDNLKH | 917 |
| SKIGSKDNIKH | 918 |
| SKIGSLDNLKH | 919 |
| SKIGSLDNIKH | 920 |
| SKIGSTGNLKH | 921 |
| SKIGSTGNIKH | 922 |
| SKIGSKGNLKH | 923 |
| SKIGSKGNIKH | 924 |
| SKIGSLGNLKH | 925 |
| SKIGSLGNIKH | 926 |

Next, to assess the potential of an unwanted T cell-mediated autoimmune response, the engineered peptides were subjected to in silico analysis to predict MHC II binding using the IEDB (Immune Epitope Database) from National Institute of Allergy and Infectious Diseases/La Jolla Immunology Institute. MHC class II binding is considered a good indicator of a sequence containing a T-cell epitope. A panel of alleles were used for MHC 11 binding prediction. Engineered peptides with a predicted half maximal inhibitory concentration (IC50) above a specified cutoff were considered to have a low probability of MHC 11 binding and were selected for further analysis.

Finally, the engineered peptides with low predicted MHC II binding were evaluated to predict if the anti-Tau MTBR antibodies that would be raised by the peptides could have unwanted cross-reactivity with other human proteins. Sequences of the engineered peptides were subjected to bioinformatic analysis against a non-redundant human proteome database to determine homology with human proteins. Engineered peptide sequences with low homology to secreted or cell-surface proteins were selected as top candidates to be used as antigens. Top candidate engineered Tau immunogenic peptides are listed in Table 2.

TABLE 2
Top candidate engineered Tau immunogenic peptides

| Engineered tau immunogenic | SEQ |
|---|---|
| peptides sequence | ID NO: |
| SKIGSTDNIKH | 916 |
| SKIGSKDNIKH | 918 |
| SKIGSLDNIKH | 920 |

Conjugation. The peptides described in the examples (Biopeptide, San Diego, CA) were coupled to CRM (CRM-bromoacetate. Fina Biosolutions, Rockville, MD) as follows:

### Example 2: Animal Immunizations

Conjugation. The peptides described in the examples (Biopeptide, San Diego, CA) were coupled to CRM (CRM-bromoacetate, Fina Biosolutions, Rockville, MD) as follows:

1M Tris HCL (pH 8.0), MilliQ DI water, 50 mM borate, 100 mM NaCl, and 5 mM EDTA pH 8.5 were sterile filtered and degassed. 1 mg of each peptide was dissolved in 0.2 mL of degassed water, then 0.1 mL, degassed Tris HCL was added, followed by 0.2 mL of the stock CRM-Bromoacetate (1 mg total) and 0.5 mL of the borate buffer. The peptide mixtures were incubated for 24 hours at 4 degrees on a nutator to provide mixing. Samples were desalted into PBS, and 5 μL was run on a 10% Tris gel to confirm conjugation.

In certain experiments, female Swiss Webster mice were injected subcutaneously at two sites with 100 μl of test article on day 0, 14, 42 and 70. Test article was prepared by combining 25 μg of test immunogen and 25 μg of QS21 adjuvant in 200 μl phosphate buffered saline (PBS). Mice were bled on day 21, 49 and 77 by nicking tails and collecting 50 μl of blood, followed by processing to serum. The peptides tested included AGHVTQAR (SEQ ID NO:453), GYTMHQD (SEQ ID NO:454), QIVYKPV (SEQ ID NO:02) and EIVYKSPV (SEQ ID NO: 141). Immunogens contained one tau peptide, a C-terminal linker and a C-terminal cysteine (i.e., -Gly-Gly-Cys-) and were coupled through the C-terminal cysteine to CRM-197 with a maleimide linkage.

In certain experiments, immunogen preparation comprised 25 μg of peptide immunogen, 25 μg QS21 and 150 μl of 0.02% Tween 80/PBS. The peptides tested included VKSKIGSTEGGC (SEQ ID NO:777), KSKIGSTEGGC (SEQ ID NO:778), SKIGSTENGGC (SEQ ID NO:779), KIGSTENLGGC (SEQ ID NO:780), IGSTENLKGGC (SEQ ID NO:781), GSTENLKHGGC (SEQ ID NO:782), STENLKHQGGC (SEQ ID NO:783), TENLKHQPGGC (SEQ ID NO:784), and ENLKHQPGGGC (SEQ ID NO:785). Female Swiss webster mice received 200 μL subcutaneously (four mice per group, each group receiving one immunogen). Mice in these experiments were injected at 0, 4 weeks and 8 weeks with bleeds taken for titer at 5 weeks. Animals were sacrificed and a terminal bleed collected at 9 weeks.

Guinea pigs were injected intramuscularly with 50 μg of a test immunogen, 25 μg QS21 in 200 μl of Addavax on day 0, 21, 49 and 77. Bleeds were done 7 days post immunization. The peptides tested included AGHVTQAR (SEQ ID NO:453), GYTMHQD (SEQ ID NO:454), QIVYKPV (SEQ ID NO:02) and EIVYKSPV (SEQ ID NO:141). Immunogens contained one tau peptide, a C-terminal linker and a C-terminal cysteine (i.e., -Gly-Gly-Cys-) and were coupled through the C-terminal cysteine to CRM-197 with a maleimide linkage.

Female Guinea Pigs were at least 5 weeks old at the start of the study having an approximate body weight of 350-500 g. Appropriate animal housing and research procedures for animal husbandry and care were conducted in an accredited facility in accordance with the guidelines of the U.S. Department of Agriculture's (USDA) and the Assessment and Accreditation of Laboratory Animal Care (AAALAC) International.

The immunogen concentration was 0.5 mg/ml. Prior to each administration of the test immunogen, approximately a 3 cm² area on each hind limb was shaved and wiped with ethanol for visualization of the injection site. Each animal received a test immunogen dose of 200 microliters (0.25 micrograms/microliter) divided into two separate sites each of 100 microliter per injection (i.e., animals received 50 μg of immunogen in 100 μl PBS+25 μg of QS-21 in 100 μl Addavax). A 25G-27G needle was inserted intramuscularly into the hind limb, approximately 0.25-0.5 cm deep, and injected at 100 microliters per site. Injection sites were rotated each administration between four separate sites per hind limb and separated by at least 2 cm.

### Example 3: Measurement of Antibody Titers

Whole blood samples were collected into clot activator tubes via jugular vein at 250-350 microliters per collection at weeks 1, 4, 8 and 12 for Guinea pigs and 50 microliters per collection at weeks 1, 3, 7 and 11 by nicking tails for mice. The maximum volume of whole blood was collected into clot activator tubes via cardiac puncture at termination on the final collection week. All blood samples were allowed to clot at room temperature for greater than 30 minutes, centrifuged ambient (approximately 20-25° C.) at 3,000 RPM for 10-15 minutes, and serum supernatant was transferred individually into clean cryovials. Serum supernatant was stored frozen at −80° C. (±12° C.).

Titer on Tau Guinea Pigs

2 μg/mi recombinant WT Tau 4R2N was coated on to the plate 100 μl per well in PBS and incubated overnight at room temperature. Plates were blocked for 1 hour with 1% BSA in PBS. Plates were aspirated and to row A 200 μl of 0.1% BSA in PBS Tween was added. In column 1, negative Guinnea pig serum was added at a 1/100 dilution while the rest of the row contained 1/100 test serums. Rows were serially diluted by 50% per step down the plate giving dilution of 1/100 to 1/12800. Wells were incubated 2 hours at room temperature and then were washed. A 1/5000 dilution of anti-Guinea Pig IgG HRP in 0.1% BSA in PBS Tween was prepared and then 100 μl added to the washed well. This incubated for 1 hour and was washed. OPD substrate was prepared using ThermoFisher OPD tablets at 1 tablet per 10 mls. ThermoFisher substrate buffer was added at 1/10 and each well had 100 μl added and was incubated for 15 minutes. 50 μl of 2N H₂SO₄ was added to stop the reaction and plates were read at 490 nm on a Molecular Devices Spectromax. Titer defined as the dilution giving 50% maximum OD and was extrapolated if it fell between dilutions.

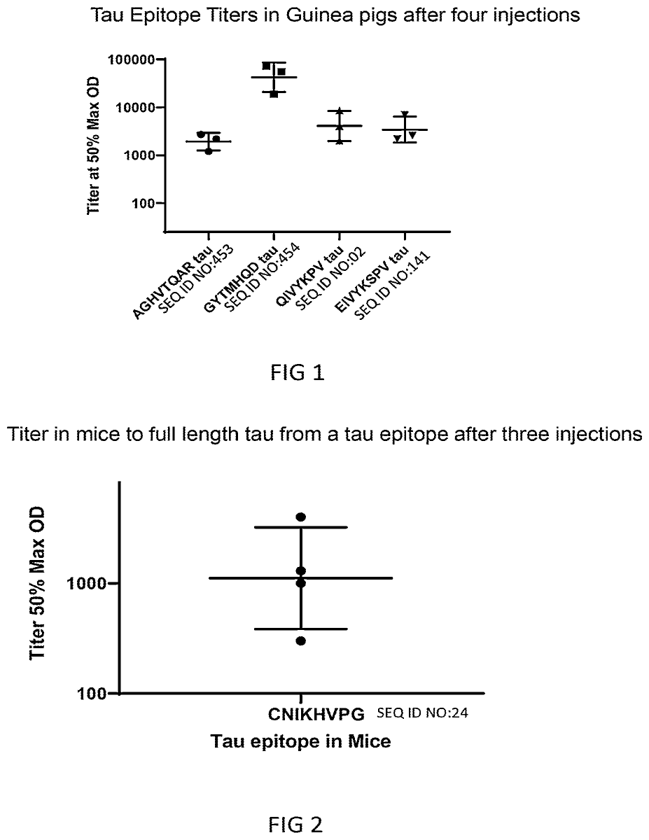

Antibody titers observed in Guinea pigs immunized as described above are shown in Table 3. Immunizations were conducted with QS21 in Addavax. The titers reported are for the bleed after the fourth injection. These results are represented in FIG. 1.

TABLE 3
Antibody titers in Guinea pigs (GP)
immunized with tau epitopes.

| Tan Epitope | SEQ | GP 1 | GP 2 | GP 2 |
|---|---|---|---|---|
| in immunogen | ID | Titer | Titer | Titer |
| AGHVTQAR | 453 | 1600 | 3200 | 1600 |
| GYTMHQD | 454 | 55000 | 73000 | 19000 |
| QIVYKPV | 02 | 4000 | 8500 | 2000 |
| EIVYSPV | 141 | 7000 | 2200 | 2600 |

Titers on Tau Mouse

Mouse serum was titered by enzyme-linked immunosorbent assay (ELISA). Plates were coated overnight at 2 μg/mL with recombinant tau (4R2N) in phosphate-buffered saline (PBS) and then blocked for 1 hour with 1% bovine serum albumin (BSA) in PBS. Plates were blocked for 1 hour with 1% BSA in PBS. Plates were aspirated and to row A 200 μl of 0.1% BSA in PBS with 0.1% Tween 20 (PBS/BSA/T) was added. Normal mouse serum was used as a negative control while known positive anti-serum from previous mouse studies was used as a positive control at the same dilutions as test serum. In column 1, negative mouse serum and positive mouse serum was added at 1/100 while the rest of the row contained 1/100 test sera. Rows were serially diluted by 50% per step down the plate giving dilution of 1/100 to 1/12800. When needed, due to high titers, 1/3 dilutions were used giving 1/100 to 1/218000 dilutions. Wells were incubated 2 hours at room temperature then were washed with TBS/Tween 20. A 1/5000 dilution of anti-mouse IgG HRP in 0.1% BSA in PBS Tween was prepared and then 100 μl added to the washed well. The reaction mixture was incubated for 1 hour and then washed with TBS/Tween 20. Antibody binding was detected with o-phenylenediamine dihydrochloride (OPD) substrate (Thermo Fisher Scientific, Waltham, MA) following manufacturer's instructions. OPD substrate was prepared using ThermoFisher OPD tablets at 1 tablet per 10 mls. ThermoFisher substrate buffer was added at 1/10 dilution and each well received 100 μl and was incubated for 15 minutes. 50 μl of 2N H₂SO₄ was added to stop the reaction and plates were read at 490 nm on a Molecular Devices Spectromax. Titer was defined as the dilution giving 50% maximum OD measurement and was extrapolated if it fell between dilutions in certain experiments. In other experiments, titer was defined as the dilution giving 4× background (defined in graphs and tables); extrapolation was used if it fell in between dilutions.

Titer Results

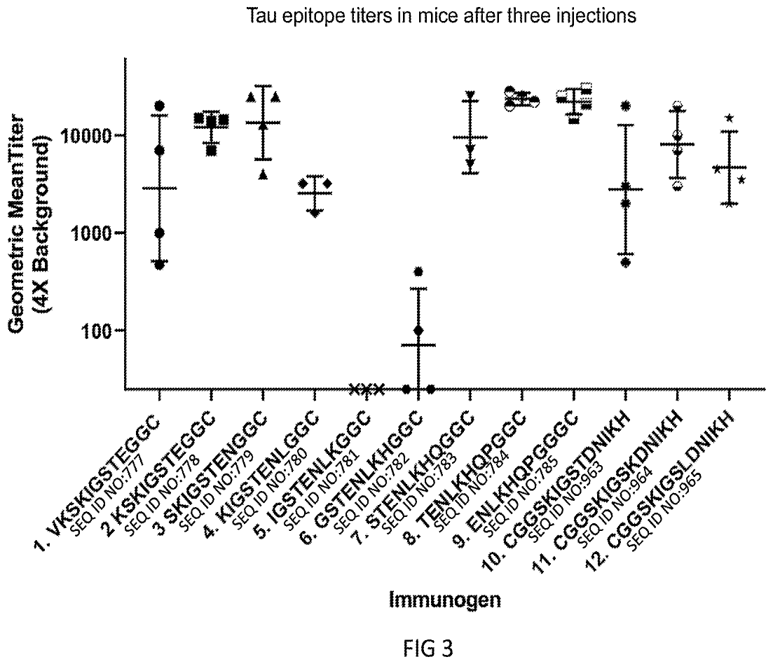

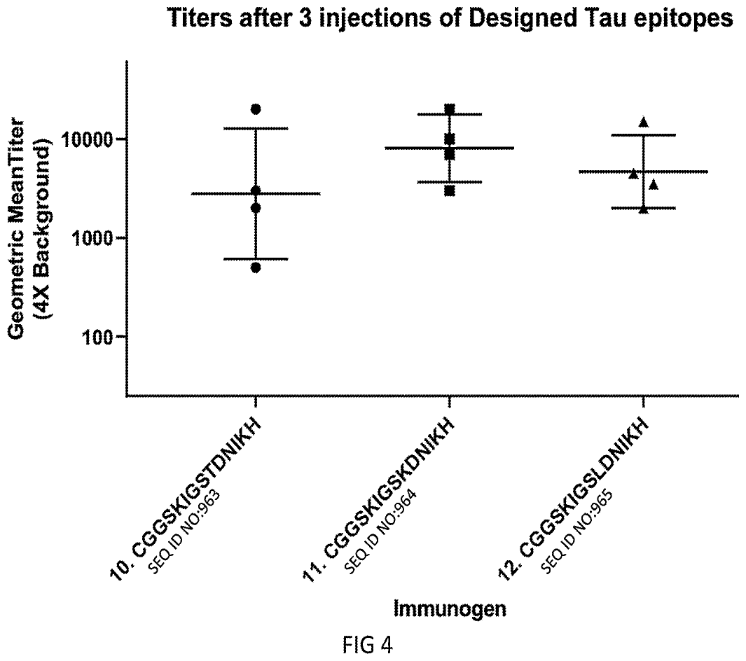

Antibody titers observed in mice immunized as described above are shown in Table 4. Immunizations were conducted with QS21. The titers reported are for the bleed after the third injection. These results are represented in FIG. 2 (sample 13), FIG. 3 (samples 1-12) and FIG. 4 (samples 10-12).

TABLE 4
Antibody titers in mice immunized with tau epitopes.

| Tau Epitope in immunogen | | mouse 1 | mouse 2 | mouse 3 | mouse 4 |
|---|---|---|---|---|---|
| 1. VKSKIGSTEGGC | QS21 0.02% | | | | |
| (SEQ ID NO: 777) | PS80 | | | | |
| | Bleed 1 | 7000 | 55000 | 35000 | 20000 |
| | Bleed 2 | 475 | 20000 | 7000 | 1000 |
| 2 KSKIGSTEGGC | QS21 0.02% | | | | |
| (SEQ ID NO: 778) | PS80 | | | | |
| | Bleed 1 | 30000 | 20000 | 10000 | 15000 |
| | Bleed 2 | 15000 | 14000 | 7000 | 14500 |
| 3 SKIGSTENGGC | QS21 0.02% | | | | |
| (SEQ ID NO: 779) | PS80 | | | | |
| | Bleed 1 | 40000 | 35000 | 15000 | 35000 |
| | Bleed 2 | 25000 | 13000 | 4000 | 25000 |
| 4. KIGSTENLGGC | QS21 0.02% | | | | |
| (SEQ ID NO: 780) | PS80 | | | | |
| | Bleed 1 | 6000 | 3000 | 1000 | dead |
| | Bleed 2 | 3200 | 1600 | 3200 | dead |
| 5. IGSTENLKGGC | QS21 0.02% | | | | |
| (SEQ ID NO: 781) | PS80 | | | | |
| | Bleed 1 | 25 | 25 | 25 | dead |
| | Bleed 2 | 25 | 25 | 25 | dead |
| 6. GSTENLKHGGC | QS21 0.02% | | | | |
| (SEQ ID NO: 782) | PS80 | | | | |
| | Bleed 1 | 200 | 25 | 25 | 12000 |
| | Bleed 2 | 100 | 25 | 25 | 400 |
| 7. STENLKHQGGC | QS21 0.02% | | | | |
| (SEQ ID NO: 783) | PS80 | | | | |
| | Bleed 1 | 20000 | 18000 | 10000 | dead |
| | Bleed 2 | 25000 | 5000 | 7000 | dead |
| 8. TENLKHQPGGC | QS21 0.02% | | | | |
| (SEQ ID NO: 784) | PS80 | | | | |
| | Bleed 1 | 40000 | 40000 | 35000 | 30000 |
| | Bleed 2 | 22000 | 28000 | 20000 | 25000 |
| 9. ENLKHQPGGGC | QS21 0.02% | | | | |
| (SEQ ID NO: 785) | PS80 | | | | |
| | Bleed 1 | 45000 | 60000 | 20000 | 45000 |
| | Bleed 2 | 21000 | 30000 | 15000 | 25000 |
| 10. CGGSKIGSTDNIKH | QS21 0.02% | | | | |
| (SEQ ID NO: 963) | PS80 | | | | |
| | Bleed 1 | 1000 | 6400 | 10000 | 15000 |
| | Bleed 2 | 500 | 2000 | 3000 | 20000 |
| 11. CGGSKIGSKDNIKH | QS21 0.02% | | | | |
| (SEQ ID NO: 964) | PS80 | | | | |
| | Bleed 1 | 10000 | 20000 | 10000 | 20000 |
| | Bleed 2 | 10000 | 7000 | 3000 | 20000 |
| 12. CGGSKIGSLDNIKH | QS21 0.02% | | | | |
| (SEQ ID NO: 965) | PS80 | | | | |
| | Bleed 1 | 7000 | 10000 | 10000 | 20000 |
| | Bleed 2 | 3500 | 2000 | 4500 | 15000 |
| 13. CNIKHVPG | | 1000 | 1300 | 300 | 4000 |
| (SEQ ID NO: 24) | | | | | |

FIG. 3 shows the results with SEQ ID NO:777 through SEQ. ID NO:785, and SEQ ID NO:963 to SEQ ID NO:965. Central peptides 4-6 (FIG. 3) did not generate high titers to tau and, thus, were not run in the heparin blocking assay of Example 4.

### Example 4: Binding of Antibody to MTBR1-MTBR4

Certain antibodies that bind to MTBR have been shown to bind to more than one MTBR region due to the homology of the various MTBR regions. Antiserum was titered on all four MTBR regions using peptides of MTBR 1-4 purchased from Anaspec (San Jose, CA).

MTBR peptide 1
(SEQ ID NO: 751)
QTAPVPMPDLKNVKSKIGSTENLKHQPGGGK
MTBR peptide 2
(SEQ ID NO: 752)
VQIINKKLDLSNVQSKCGSKDNIKHVPGGGS
MTBR peptide 3
(SEQ ID NO: 753)
VQIVYKPVDLSKVTSKCGSLGNIHHKPGGGQ
MTBR peptide 4
(SEQ ID NO: 754)
VEVKSEKLDFKDRVQSKIGSLDNITHVPGGGN

Mouse serum was again titered by enzyme-linked immunosorbent assay (ELISA). Plates were coated overnight at 2 μg/mL with each the various MTBR peptides in phosphate-buffered saline (PBS) and then blocked 1 hour with 1% bovine serum albumin (BSA) in PBS. Normal mouse serum was used as a negative control. Bleeds were diluted in PBS/0.1% BSA/0.1% Tween 20 (PBS/BSA/T) starting at 1/100 and serially diluted 1:2 down the plate. Plates were washed with TBS/Tween 20, and goat anti-mouse immunoglobulin G (IgG) (heavy+light chains) horseradish peroxidase (HRP) (from ThermoFisher) was added at a 1/5000 dilution and incubated for 1 hour at room temperature. Plates were washed in TBS/Tween 20, and antibody binding was detected with o-phenylenediamine dihydrochloride (OPD) substrate (Thermo Fisher Scientific, Waltham, MA) following manufacturer's instructions. Plates were read at 490 nM on a Molecular Devices Spectromax, and titer was defined as the dilution giving 4× background (defined in graphs and tables); extrapolation was used if it fell in between dilutions.

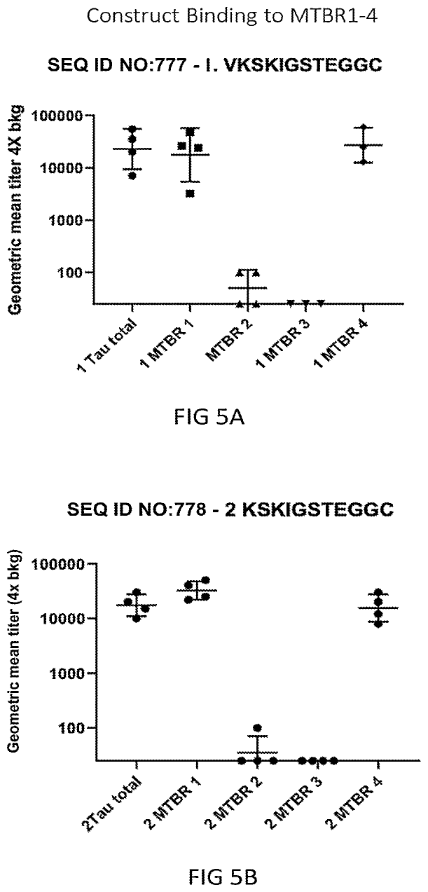

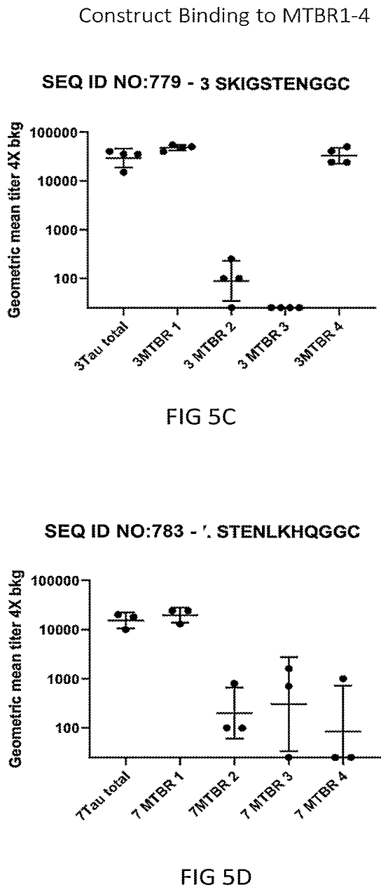

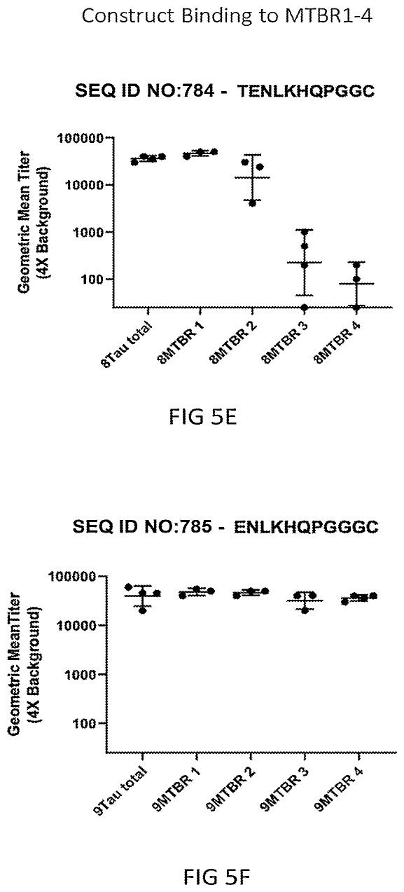

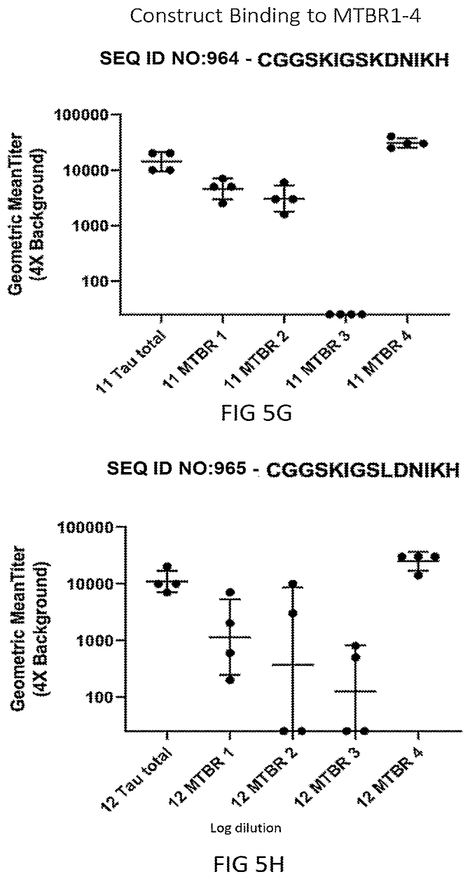

Peptide binding is showing in FIG. 5(A)-5(H). Overall, VKSKIGSTEGGC (SEQ ID NO:777; FIG. 5(A)). KSKIGSTEGGC (SEQ ID NO:778; FIG. 5(B)), SKIGSTENGGC (SEQ ID NO:779; FIG. 5(C)) bound strongly to MTBR 1 and 4. STENLKHQGGC (SEQ ID NO:783; FIG. 5(D)) bound strongly to only MTBR 1. TENLKHQPGGC (SEQ ID NO:784; FIG. 5(E)) bound strongly to MTBR 1 and 2, while ENLKHQPGGGC (SEQ ID NO:785; FIG. 5(F)) bound to all four MTBR.

### Example 5: Blocking of Tau Binding to Heparin

As a potential surrogate marker for the ability of the serum to block uptake of tau into cells, an ELISA measuring the blocking of tau binding to heparin plates was developed. Recombinant tau was biotinylated in-house. Heparin coated plates (Bioworld, Dublin, OH) were blocked with 2% BSA/PBS for 1 hour. In a separate deep-well polypropylene 96 well plate (ThermoFisher), serum was diluted from 1/50 to 1/6400 in 2% BSA/PBS, in 60 μl total volume. To this, 60 μl of 200 ng/ml biotinylated tau in 2% BSA/PBS was added for a final concentration of serum 1/100-1/12800, with tau at 100 ng/ml. The mixture of serum and tau was incubated for 2 hours, then 100 μl/well was transferred to the blocked heparin plates and incubated 1 hour. Plates were washed in 0.1% Tween 20/TBS and goat anti-mouse immunoglobulin G (IgG) (heavy+light chains) horseradish peroxidase (HRP) (ThermoFisher) was added at a 1/5000 dilution and incubated for 1 hour at room temperature. Plates were washed in TBS/Tween 20, and 100 μl ThennoFisher TMB was added and incubated for 8 minutes and then stopped with H₂SO₄ and read at 450 nm.

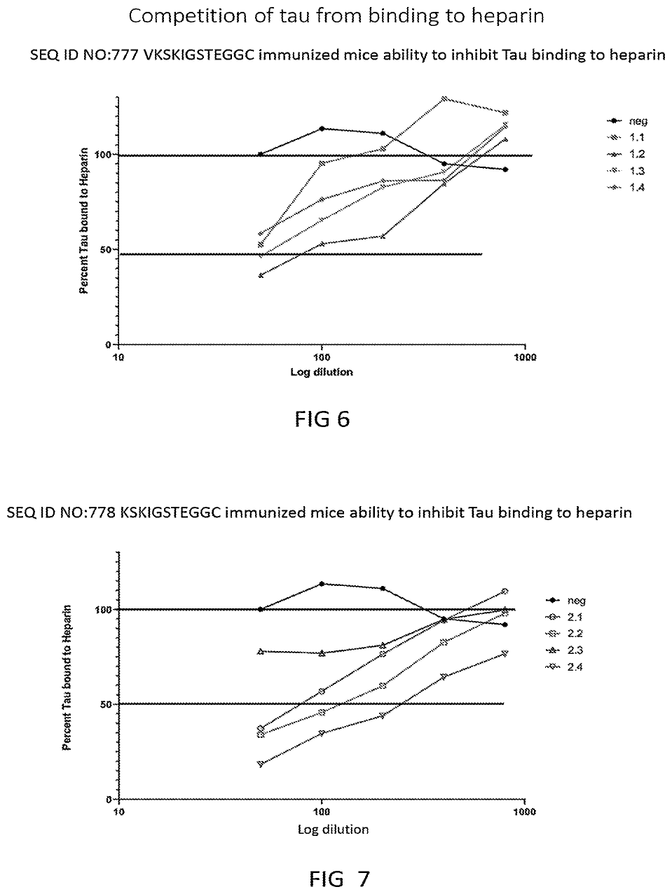

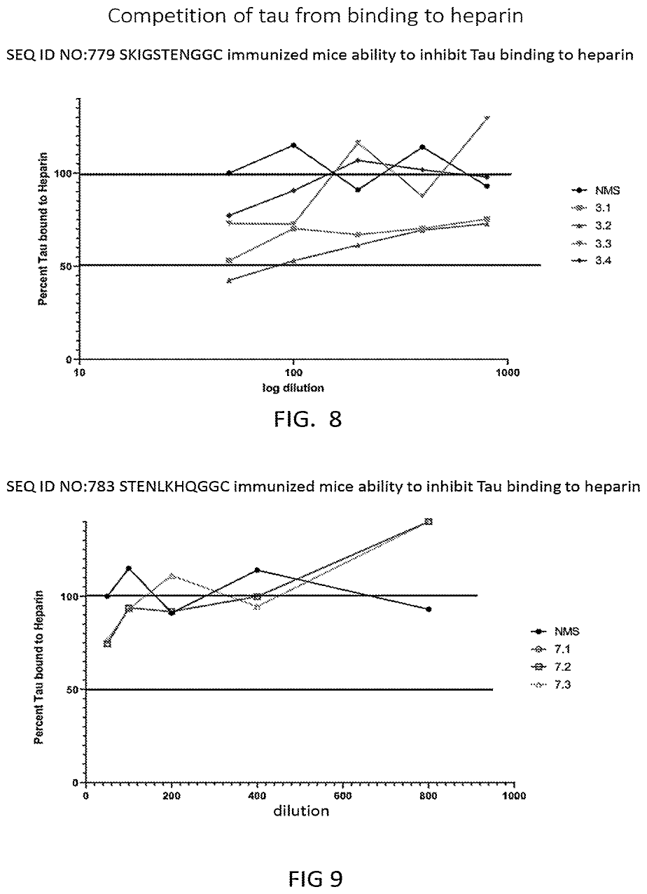

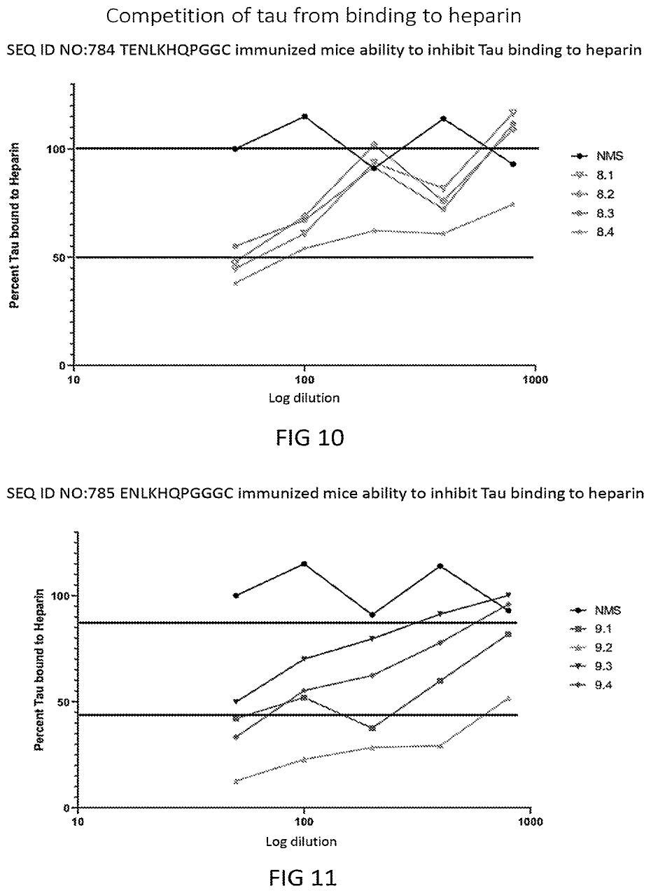

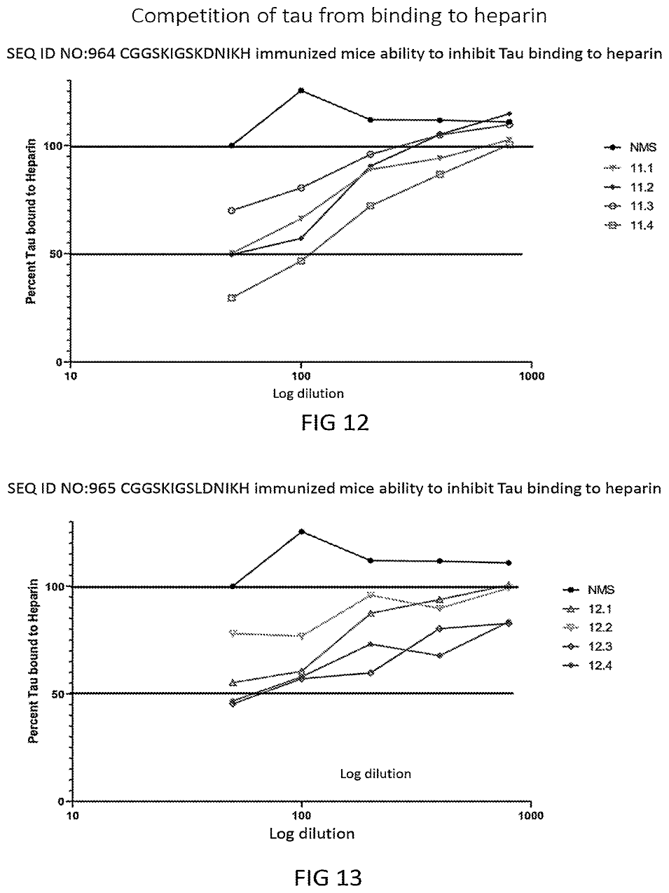

FIGS. 6-13 show blocking of tau binding to heparin. FIG. 6 shows results for VKSKIGSTEGGC (SEQ ID NO:777); FIG. 7 shows results for KSKIGSTEGGC (SEQ ID NO:778); FIG. 8 shows results for SKIGSTENGGC (SEQ ID NO:779); FIG. 9 shows results for STENLKHQGGC (SEQ ID NO:783); FIG. 10 shows results for TENLKHQPGGC (SEQ ID NO:784); FIG. 11 shows results for ENLKHQPGGGC (SEQ ID NO:785): FIG. 12 shows results for CGGSKIGSKDNIKH (SEQ ID NO:964); and FIG. 13 shows results for CGGSKIGSLDNIKH (SEQ ID NO:965).

Quantitative measurement of the inhibition of tau binding to heparin is shown in Table 5 below.

TABLE 5

| | |
|---|---|
| | neg/NMS |
| dilution | control |

(1) VKSKIGSTEGGC (SEQ ID NO: 777), animals 1-4

| | | 1.1 | 1.2 | 1.3 | 1.4 |
|---|---|---|---|---|---|
| 100 | 100.00 | 52.52 | 36.48 | 46.34 | 58.40 |
| 200 | 113.45 | 95.26 | 52.94 | 65.31 | 76.23 |
| 400 | 111.02 | 102.94 | 57.01 | 82.73 | 86.02 |
| 800 | 95.01 | 129.16 | 84.63 | 90.68 | 86.36 |
| 1600 | 91.98 | 121.81 | 108.05 | 115.51 | 114.49 |

(2) KSKIGSTEGGC (SEQ ID NO: 778), animals 1-4

| | | 2.1 | 2.2 | 2.3 | 2.4 |
|---|---|---|---|---|---|
| 100 | 100.00 | 37.28 | 34.00 | 77.89 | 18.32 |
| 200 | 113.45 | 56.86 | 45.67 | 76.90 | 34.63 |
| 400 | 111.02 | 76.47 | 59.74 | 81.08 | 43.94 |
| 800 | 95.01 | 94.25 | 82.62 | 94.75 | 64.34 |
| 1600 | 91.98 | 109.52 | 97.98 | 99.67 | 76.62 |

(3) SKIGSTENGGC (SEQ ID NO: 779), animals 1-4

| | | 3.1 | 3.2 | 3.3 | 3.4 |
|---|---|---|---|---|---|
| 100 | 100.000 | 52.99 | 42.46 | 72.94 | 77.24 |
| 200 | 115.000 | 70.24 | 52.95 | 72.71 | 90.63 |
| 400 | 91.000 | 67.02 | 61.32 | 116.24 | 106.92 |
| 800 | 114.000 | 70.32 | 69.47 | 87.73 | 101.75 |
| 1600 | 93.000 | 75.35 | 72.74 | 129.27 | 97.90 |

(4) STENLKHQGGC (SEQ ID NO: 783), animals 1-3

| | | 7.1 | 7.2 | 7.3 |
|---|---|---|---|---|
| 100 | 100.000 | 74.48 | 74.48 | 76.38 |
| 200 | 115.000 | 93.77 | 93.77 | 93.15 |
| 400 | 91.000 | 91.77 | 91.77 | 111.09 |
| 800 | 114.000 | 99.83 | 99.83 | 94.48 |
| 1600 | 93.000 | 140.00 | 140.00 | 140.00 |

(8) TENLKHQPGGC (SEQ ID NO: 784), animals 1-4 and
(9) ENLKHQPGGGC (SEQ ID NO: 785), animals 1-2

| | | 8.1 | 8.2 | 8.3 | 8.4 | 9.1 | 9.2 |
|---|---|---|---|---|---|---|---|
| 100 | 100.000 | 44.74 | 47.87 | 55.01 | 38.00 | 42.14 | 12.67 |
| 200 | 115.000 | 60.90 | 68.96 | 67.15 | 54.05 | 52.02 | 22.85 |
| 400 | 91.000 | 93.69 | 101.92 | 91.70 | 62.21 | 37.51 | 28.48 |
| 800 | 114.000 | 81.74 | 75.99 | 72.19 | 60.93 | 59.88 | 29.26 |
| 1600 | 93.000 | 116.63 | 109.21 | 111.64 | 74.37 | 81.91 | 51.70 |

(9) ENLKHQPGGGC (SEQ ID NO: 785), animals 3-4 and
(11) CGGSKIGSKDNIKH (SEQ ID NO: 964), animals 1-4

| | | 9.3 | 9.4 | 11.1 | 11.2 | 11.3 | 11.4 |
|---|---|---|---|---|---|---|---|
| 100 | 100.00 | 49.87 | 33.33 | 50.19 | 49.57 | 69.96 | 29.61 |
| 200 | 125.44 | 70.11 | 55.25 | 66.19 | 57.12 | 80.40 | 46.60 |
| 400 | 111.94 | 79.63 | 62.35 | 88.92 | 90.49 | 95.91 | 72.05 |
| 800 | 111.72 | 91.34 | 77.86 | 94.14 | 105.11 | 104.90 | 86.62 |
| 1600 | 110.91 | 100.14 | 95.96 | 102.60 | 114.72 | 109.67 | 100.38 |

(12) CGGSKIGSLDNIKH (SEQ ID NO: 965), animals 1-4

| | | 12.1 | 12.2 | 12.3 | 12.4 |
|---|---|---|---|---|---|
| 100 | 100.00 | 55.15 | 78.01 | 45.33 | 46.65 |
| 200 | 125.44 | 60.51 | 76.83 | 57.06 | 57.93 |
| 400 | 111.94 | 87.44 | 95.86 | 59.71 | 73.17 |
| 800 | 111.72 | 93.85 | 89.73 | 80.34 | 67.75 |
| 1600 | 110.91 | 100.58 | 99.20 | 82.68 | 83.47 |

### Example 6: Staining of Alzheimer's Brain Tissue with Sera from Mice and Guinea Pigs Immunized with Vaccines as Disclosed Herein

Autopsy blocks of fresh frozen human brain tissue (˜0.5 g) were embedded in optimal cutting temperature compound (OCT compound) and cut using a cryostat to generate 10 μm sections. The sections are placed into a solution of glucose oxidase and beta D-glucose, in the presence of sodium azide, to block endogenous peroxidase.

Once tissue sections were prepared, the staining with the specified mouse immune sera was carried out at 1:500 in 5% goat serum with 0.25% triton for 1 hour at RT. To image the binding to plagues and tangles, Biotin-SP-Conjugated Goat anti mouse IgG from Jackson (Lot #115-065-166) at 1:200 dilution was incubated with the sections. A DAKO DAB Detection Kit was used according to the manufacturer's instructions, and staining was processed using an automated Leica Bond Stainer. The results indicate that sera from mice immunized with a vaccine as disclosed herein comprise antibodies specific to tau in human brain tissue of Alzheimer's patients.

For Guinea pigs immunized with a vaccine as disclosed herein, once tissue sections were prepared, the staining with the specified Guinea pig sera was carried out at two dilutions (1:300 and 1:1500), using a rabbit anti-guinea pig secondary antibody and a DAKO DAB Detection Kit as per the manufacturer's instructions. The staining was processed using an automated Leica Bond Stainer. The results indicated whether sera from Guinea pigs immunized with a vaccine as disclosed herein comprise antibodies specific to tau in human brain tissue of Alzheimer's patients.

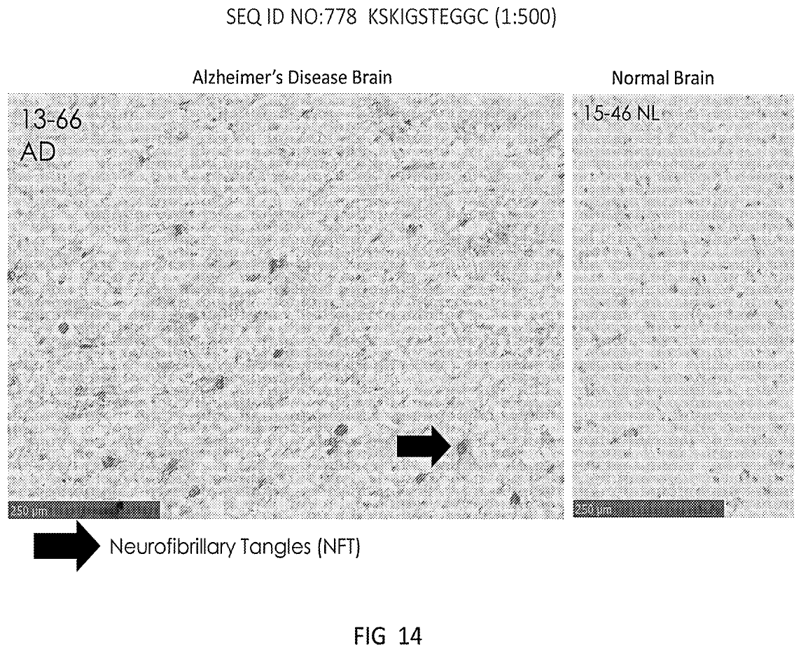

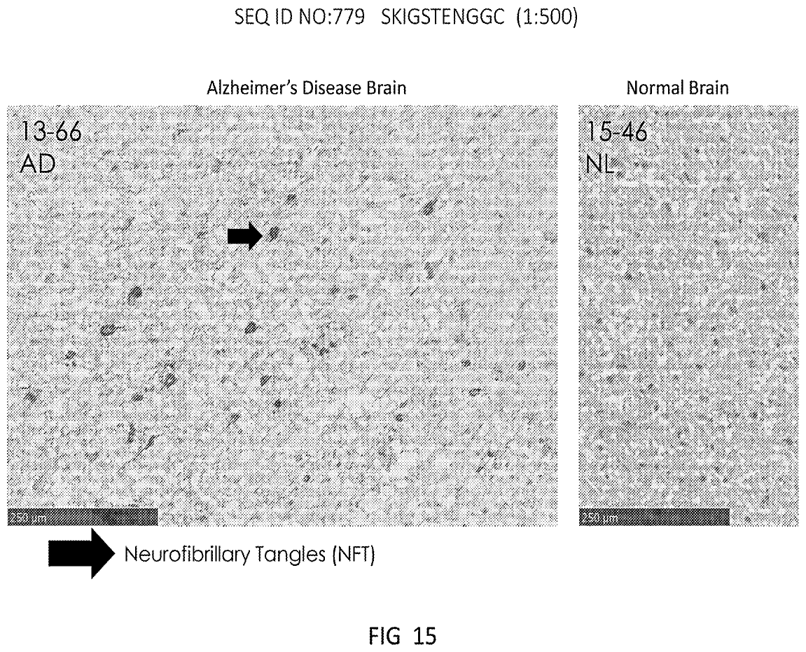

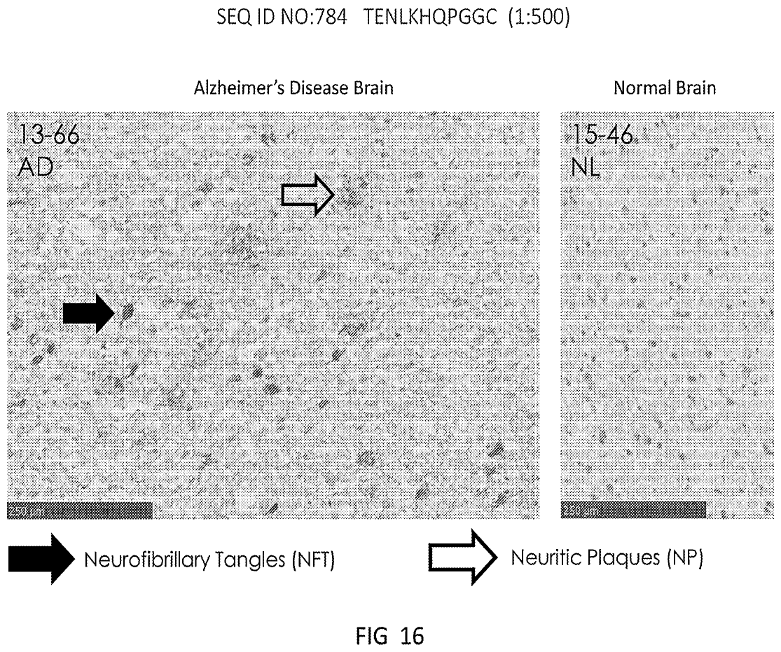

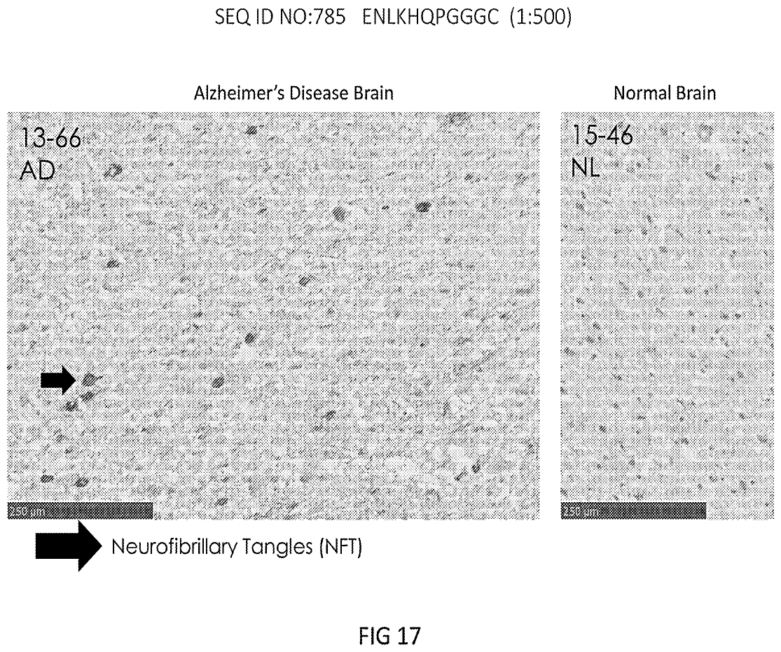

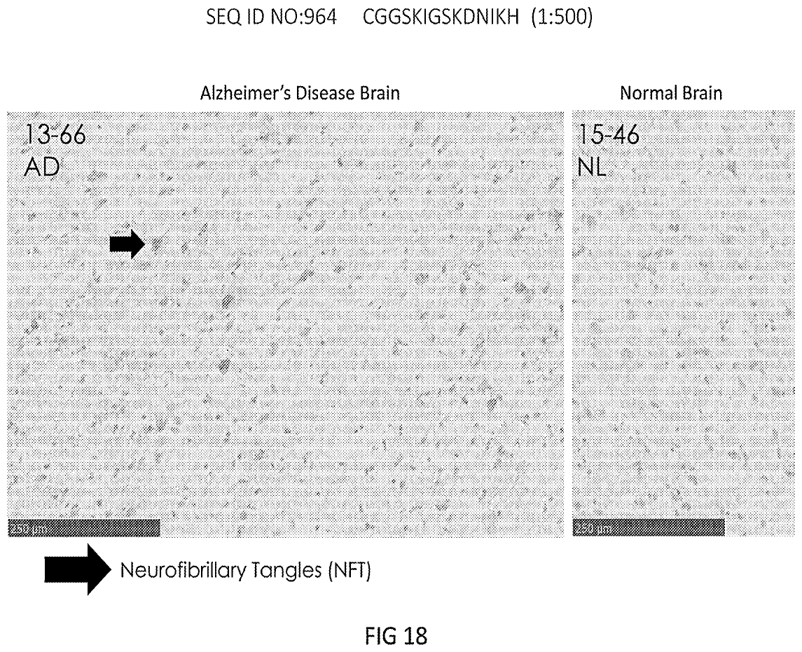

Table 6 shows a summary of the ability of the mouse sera to bind to pathological tau in Alzheimer patient brains. FIGS. 14-18 are examples of that binding. ND indicates not detected; + signs indicate binding.

TABLE 6
Ranking of mouse sera binding to
pathological Tau in Alzheimer's brain

| Construct | Mouse 1 | Mouse 2 | Mouse 3 | Mouse 4 |
|---|---|---|---|---|
| VKSKIGSTEGGC | + | +++ | +++ | +++ |
| (SEQ ID NO: 777) | | | | |
| KSKIGSTEGGC | ++ | +++ | ++ | + |
| (SEQ ID NO: 778) | | | | |
| SKIGSTENGGC | +++ | +++ | + | ++ |
| (SEQ ID NO: 779) | | | | |
| KIGSTENLGGC | + | ++ | + | ND |
| (SEQ ID NO: 780) | | | | |
| IGSTENLKGGC | Astrocyte | − | − | ND |
| (SEQ ID NO: 781) | only | | | |
| GSTENLKHGGC | − | − | − | − |
| (SEQ ID NO: 782) | | | | |
| STENLKHQGGC | + | − | + | ND |
| (SEQ ID NO: 783) | | | | |
| TENLKHQPGGC | +++ | +++ | ++ | +++ |
| (SEQ ID NO: 784) | | | | |
| ENLKHQPGGGC | ++ | +++ | +++ | ++ |
| (SEQ ID NO: 785) | Astrocytes | | | |
| CGGSKIGSTDNIKH | −A | − | − | − |
| (SEQ ID NO: 963) | | | | |
| CGGSKIGSKDNIKH | + | −A | − | −A |
| (SEQ ID NO: 964) | | | | |
| CGGSKIGSLDNIKH | −A | −A | + | −A |
| (SEQ ID NO: 965) | | | | |

−: negative
+: weak
++: moderate
+++: strong
A: astrocyte staining

### Example 7: Mice Vaccinated with Tau Antigens Produce Titers to Tau

Swiss Webster Female mice are injected on day 0, 14 and 28 with 25 μg of a tau peptide immunogen (e.g., SEQ IDs herein) and 25 μg QS21 (Desert King) in PBS total 200 μl/injection. Each mouse receives 200 μl subcutaneously. Mice are bled on day 21 and 35.

Although various specific embodiments of the present invention have been described herein, it is to be understood that the invention is not limited to those precise embodiments and that various changes or modifications can be affected therein by one skilled in the art without departing from the scope and spirit of the invention. In addition, in each of the embodiments of the peptide described herein, the peptide may comprise, consist, or consist essentially of the recited sequences.

In each of the embodiments of the peptide described herein, the peptide may comprise, consist, or consist essentially of the recited sequences. Thus, incorporated in this disclosure (see Table 7) are the following sequences that can be part of the compositions comprising, consisting of or consisting essentially of an tau peptide as disclosed herein.

TABLE 7
SEQUENCES

SEQ ID NO: 01 - TAU P10636-8
MAEPRQEFEV MEDHAGTYGL GDRKDQGGYT MHQDQEGDTD AGLKESPLQT PTEDGSEEPG
SETSDAKSTP TAEDVTAPLV DEGAPGKQAA AQPHTEIPEG TTAEEAGIGD TPSLEDEAAG
HVTQARMVSK SKDGTGSDDK KAKGADGKTK IATPRGAAPP GQKGQANATR IPAKTPPAPK
TPPSSGEPPK SGDRSGYSSP GSPGTPGSRS RTPSLPTPPT REPKKVAVVR TPPKSPSSAK
SRLQTAPVPM PDLKNVKSKI GSTENLKHQP GGGKVQIINK KLDLSNVQSK CGSKDNIKHV
PGGGSVQIVY KPVDLSKVTS KCGSLGNIHH KPGGGQVEVK SEKLDFKDRV QSKIGSLDNI
THVPGGGNKK IETHKLTFRE NAKAKTDHGA EIVYKSPVVS GDTSPRHLSN VSSTGSIDMV
DSPQLATLAD EVSASLAKQG L
QIVYKPV (SEQ ID NO: 02)
QIVYKP (SEQ ID NO: 03)
NIKHVP (SEQ ID NO: 04)
NIKHVPG (SEQ ID NO: 05)
HVPGGG (SEQ ID NO: 06)
HVPGG (SEQ ID NO: 07)
HKPGGG (SEQ ID NO: 08)
HKPGG (SEQ ID NO: 09)
KHVPGGG (SEQ ID NO: 10)
KHVPGG (SEQ ID NO: 11)
HQPGGG (SEQ ID NO: 12)
HQPGG (SEQ ID NO: 13)
VQIINK (SEQ ID NO: 14)
VQIINKK (SEQ ID NO: 15)
VQIINKKL (SEQ ID NO: 16)
QIINK (SEQ ID NO: 17)
QIINKK (SEQ ID NO: 18)
QIINKKL (SEQ ID NO: 19)
QIVYKSV (SEQ ID NO: 20)
EIVYKSV (SEQ ID NO: 21)
EIVYKPV (SEQ ID NO: 22)
CNIKHVP (SEQ ID NO: 23)
CNIKHVPG (SEQ ID NO: 24)
EIVYKSP (SEQ ID NO: 25)
IVYKSPV (SEQ ID NO: 26)
IVYK (SEQ ID NO: 27)
VPGGGSVQIV (SEQ ID NO: 28)
PGGGSVQIV (SEQ ID NO: 29)
GGGSVQIV (SEQ ID NO: 30)
GGSVQIV (SEQ ID NO: 31)
GSVQIV (SEQ ID NO: 32)
SVQIV (SEQ ID NO: 33)
VQIV (SEQ ID NO: 34)
QIV (SEQ ID NO: 35)
PGGGSVQIVY (SEQ ID NO: 36)
GGGSVQIVY (SEQ ID NO: 37)
GGSVQIVY (SEQ ID NO: 38)
GSVQIVY (SEQ ID NO: 39)
SVQIVY (SEQ ID NO: 40)
VQIVY (SEQ ID NO: 41)
QIVY (SEQ ID NO: 42)
IVY (SEQ ID NO: 43)
GGGSVQIVYK (SEQ ID NO: 44)
GGSVQIVYK (SEQ ID NO: 45)
GSVQIVYK (SEQ ID NO: 46)
SVQIVYK (SEQ ID NO: 47)
VQIVYK (SEQ ID NO: 48)
QIVYK (SEQ ID NO: 49)
VYK (SEQ ID NO: 50)
GGSVQIVYKP (SEQ ID NO: 51)
GSVQIVYKP (SEQ ID NO: 52)
SVQIVYKP (SEQ ID NO: 53)
VQIVYKP (SEQ ID NO: 54)
IVYKP (SEQ ID NO: 55)
VYKP (SEQ ID NO: 56)
YKP (SEQ ID NO: 57)
GSVQIVYKPV (SEQ ID NO: 58)
SVQIVYKPV (SEQ ID NO: 59)
VQIVYKPV (SEQ ID NO: 60)
IVYKPV (SEQ ID NO: 61)
VYKPV (SEQ ID NO: 62)
YKPV (SEQ ID NO: 63)
KPV (SEQ ID NO: 64)
SVQIVYKPVD (SEQ ID NO: 65)
VQIVYKPVD (SEQ ID NO: 66)
QIVYKPVD (SEQ ID NO: 67)
IVYKPVD (SEQ ID NO: 68)
VYKPVD (SEQ ID NO: 69)
YKPVD (SEQ ID NO: 70)
KPVD (SEQ ID NO: 71)
PVD (SEQ ID NO: 72)
VQIVYKPVDL (SEQ ID NO: 73)
QIVYKPVDL (SEQ ID NO: 74)
IVYKPVDL (SEQ ID NO: 75)
VYKPVDL (SEQ ID NO: 76)
YKPVDL (SEQ ID NO: 77)
KPVDL (SEQ ID NO: 78)
PVDL (SEQ ID NO: 79)
VDL (SEQ ID NO: 80)
QIVYKPVDLS (SEQ ID NO: 81)
IVYKPVDLS (SEQ ID NO: 82)
VYKPVDLS (SEQ ID NO: 83)
YKPVDLS (SEQ ID NO: 84)
KPVDLS (SEQ ID NO: 85)
PVDLS (SEQ ID NO: 86)
VDLS (SEQ ID NO: 87)
IVYKPVDLSK (SEQ ID NO: 88)
VYKPVDLSK (SEQ ID NO: 89)
YKPVDLSK (SEQ ID NO: 90)
KPVDLSK (SEQ ID NO: 91)
PVDLSK (SEQ ID NO: 92)
VDLSK (SEQ ID NO: 93)
VYKPVDLSKV (SEQ ID NO: 94)
YKPVDLSKV (SEQ ID NO: 95)
KPVDLSKV (SEQ ID NO: 96)
PVDLSKV (SEQ ID NO: 97)
VDLSKV (SEQ ID NO: 98)
YKPVDLSKVT (SEQ ID NO: 99)
KPVDLSKVT (SEQ ID NO: 100)
PVDLSKVT (SEQ ID NO: 101)
VDLSKVT (SEQ ID NO: 102)
AKTDHGAEIV (SEQ ID NO: 103)
KTDHGAEIV (SEQ ID NO: 104)
TDHGAEIV (SEQ ID NO: 105)
DHGAEIV (SEQ ID NO: 106)
HGAEIV (SEQ ID NO: 107)
GAEIV (SEQ ID NO: 108)
AEIV (SEQ ID NO: 109)
EIV (SEQ ID NO: 110)
KTDHGAEIVY (SEQ ID NO: 111)
TDHGAEIVY (SEQ ID NO: 112)
DHGAEIVY (SEQ ID NO: 113)
HGAEIVY (SEQ ID NO: 114)
GAEIVY (SEQ ID NO: 115)
AEIVY (SEQ ID NO: 116)
EIVY (SEQ ID NO: 117)
TDHGAEIVYK (SEQ ID NO: 118)
DHGAEIVYK (SEQ ID NO: 119)
HGAEIVYK (SEQ ID NO: 120)
GAEIVYK (SEQ ID NO: 121)
AEIVYK (SEQ ID NO: 122)
EIVYK (SEQ ID NO: 123)
DHGAEIVYKS (SEQ ID NO: 124)
HGAEIVYKS (SEQ ID NO: 125)
GAEIVYKS (SEQ ID NO: 126)
AEIVYKS (SEQ ID NO: 127)
EIVYKS (SEQ ID NO: 128)
IVYKS (SEQ ID NO: 129)
VYKS (SEQ ID NO: 130)
YKS (SEQ ID NO: 131)
HGAEIVYKSP (SEQ ID NO: 132)
GAEIVYKSP (SEQ ID NO: 133)
AEIVYKSP (SEQ ID NO: 134)
IVYKSP (SEQ ID NO: 135)
VYKSP (SEQ ID NO: 136)
YKSP (SEQ ID NO: 137)
KSP (SEQ ID NO: 138)
GAEIVYKSPV (SEQ ID NO: 139)
AEIVYKSPV (SEQ ID NO: 140)
EIVYKSPV (SEQ ID NO: 141)
VYKSPV (SEQ ID NO: 142)
YKSPV (SEQ ID NO: 143)
KSPV (SEQ ID NO: 144)
SPV (SEQ ID NO: 145)
AEIVYKSPVV (SEQ ID NO: 146)
EIVYKSPVV (SEQ ID NO: 147)
IVYKSPVV (SEQ ID NO: 148)
VYKSPVV (SEQ ID NO: 149)
YKSPVV (SEQ ID NO: 150)
KSPVV (SEQ ID NO: 151)
SPVV (SEQ ID NO: 152)
PVV (SEQ ID NO: 153)
EIVYKSPVVS (SEQ ID NO: 154)
IVYKSPVVS (SEQ ID NO: 155)
VYKSPVVS (SEQ ID NO: 156)
YKSPVVS (SEQ ID NO: 157)
KSPVVS (SEQ ID NO: 158)
SPVVS (SEQ ID NO: 159)
VVS (SEQ ID NO: 160)
IVYKSPVVSG (SEQ ID NO: 161)
VYKSPVVSG (SEQ ID NO: 162)
YKSPVVSG (SEQ ID NO: 163)
KSPVVSG (SEQ ID NO: 164)
SPVVSG (SEQ ID NO: 165)
PVVSG (SEQ ID NO: 166)
VYKSPVVSGD (SEQ ID NO: 167)
YKSPVVSGD (SEQ ID NO: 168)
KSPVVSGD (SEQ ID NO: 169)
SPVVSGD (SEQ ID NO: 170)
PVVSGD (SEQ ID NO: 171)
VVSGD (SEQ ID NO: 172)
YKSPVVSGDT (SEQ ID NO: 173)
KSPVVSGDT (SEQ ID NO: 174)
SPVVSGDT (SEQ ID NO: 175)
PVVSGDT (SEQ ID NO: 176)
KSPVVSGDTS (SEQ ID NO: 177)
SPVVSGDTS (SEQ ID NO: 178)
PVVSGDTS (SEQ ID NO: 179)
VVSGDTS (SEQ ID NO: 180)
SPVVSGDTSP (SEQ ID NO: 181)
PVVSGDTSP (SEQ ID NO: 182)
VVSGDTSP (SEQ ID NO: 183)
PVVSGDTSPR (SEQ ID NO: 184)
HQPGGGKVQI (SEQ ID NO: 185)
QPGGGKVQI (SEQ ID NO: 186)
PGGGKVQI (SEQ ID NO: 187)
GGGKVQI (SEQ ID NO: 188)
GGKVQI (SEQ ID NO: 189)
GKVQI (SEQ ID NO: 190)
KVQI (SEQ ID NO: 191)
VQI (SEQ ID NO: 192)
QPGGGKVQII (SEQ ID NO: 193)
PGGGKVQII (SEQ ID NO: 194)
PGGGKVQII (SEQ ID NO: 195)
GGGKVQII (SEQ ID NO: 196)
GGKVQII (SEQ ID NO: 197)
GKVQII (SEQ ID NO: 198)
KVQII (SEQ ID NO: 199)
VQII (SEQ ID NO: 200)
QII (SEQ ID NO: 201)
PGGGKVQIIN (SEQ ID NO: 202)
GGGKVQIIN (SEQ ID NO: 203)
GGKVQIIN (SEQ ID NO: 204)
GKVQIIN (SEQ ID NO: 205)
KVQIIN (SEQ ID NO: 206)
VQIIN (SEQ ID NO: 207)
QIIN (SEQ ID NO: 208)
IIN (SEQ ID NO: 209)
GGGKVQIINK (SEQ ID NO: 210)
GGKVQIINK (SEQ ID NO: 211)
GKVQIINK (SEQ ID NO: 212)
KVQIINK (SEQ ID NO: 213)
IINK (SEQ ID NO: 214)
INK (SEQ ID NO: 215)
GGKVQIINKK (SEQ ID NO: 216)
GKVQIINKK (SEQ ID NO: 217)
KVQIINKK (SEQ ID NO: 218)
IINKK (SEQ ID NO: 219)
INKK (SEQ ID NO: 220)
NKK (SEQ ID NO: 221)
GKVQIINKKL (SEQ ID NO: 222)
KVQIINKKL (SEQ ID NO: 223)
IINKKL (SEQ ID NO: 224)
INKKL (SEQ ID NO: 225)
NKKL (SEQ ID NO: 226)
KKL (SEQ ID NO: 227)
KVQIINKKLD (SEQ ID NO: 228)
VQIINKKLD (SEQ ID NO: 229)
QIINKKLD (SEQ ID NO: 230)
IINKKLD (SEQ ID NO: 231)
INKKLD (SEQ ID NO: 232)
NKKLD (SEQ ID NO: 233)
KKLD (SEQ ID NO: 234)
VQIINKKLDL (SEQ ID NO: 235)
QIINKKLDL (SEQ ID NO: 236)
IINKKLDL (SEQ ID NO: 237)
INKKLDL (SEQ ID NO: 238)
NKKLDL (SEQ ID NO: 239)
KKLDL (SEQ ID NO: 240)
QIINKKLDLS (SEQ ID NO: 241)
IINKKLDLS (SEQ ID NO: 242)
INKKLDLS (SEQ ID NO: 243)
NKKLDLS (SEQ ID NO: 244)
KKLDLS (SEQ ID NO: 245)
IINKKLDLSN (SEQ ID NO: 246)
INKKLDLSN (SEQ ID NO: 247)
NKKLDLSN (SEQ ID NO: 248)
KKLDLSN (SEQ ID NO: 249)
INKKLDLSNV (SEQ ID NO: 250)
NKKLDLSNV (SEQ ID NO: 251)
KKLDLSNV (SEQ ID NO: 252)
NKKLDLSNVQ (SEQ ID NO: 253)
KKLDLSNVQ (SEQ ID NO: 254)
KKLDLSNVQS (SEQ ID NO: 255)
SKCGSKDNIK (SEQ ID NO: 256)
KCGSKDNIK (SEQ ID NO: 257)
CGSKDNIK (SEQ ID NO: 258)
SKDNIK (SEQ ID NO: 259)
KDNIK (SEQ ID NO: 260)
DNIK (SEQ ID NO: 261)
NIK (SEQ ID NO: 262)
KCGSKDNIKH (SEQ ID NO: 263)
CGSKDNIKH (SEQ ID NO: 264)
SKDNIKH (SEQ ID NO: 265)
KDNIKH (SEQ ID NO: 266)
DNIKH (SEQ ID NO: 267)
NIKH (SEQ ID NO: 268)
IKH (SEQ ID NO: 269)
CGSKDNIKHV (SEQ ID NO: 270)
SKDNIKHV (SEQ ID NO: 271)
KDNIKHV (SEQ ID NO: 272)
DNIKHV (SEQ ID NO: 273)
NIKHV (SEQ ID NO: 274)
IKHV (SEQ ID NO: 275)
KHV (SEQ ID NO: 276)
SKDNIKHVP (SEQ ID NO: 277)
KDNIKHVP (SEQ ID NO: 278)
DNIKHVP (SEQ ID NO: 279)
IKHVP (SEQ ID NO: 280)
KHVP (SEQ ID NO: 281)
HVP (SEQ ID NO: 282)
SKDNIKHVPG (SEQ ID NO: 283)
KDNIKHVPG (SEQ ID NO: 284)
DNIKHVPG (SEQ ID NO: 285)
IKHVPG (SEQ ID NO: 286)
KHVPG (SEQ ID NO: 287)
HVPG (SEQ ID NO: 288)
VPG (SEQ ID NO: 289)
KDNIKHVPGG (SEQ ID NO: 290)
DNIKHVPGG (SEQ ID NO: 291)
NIKHVPGG (SEQ ID NO: 292)
IKHVPGG (SEQ ID NO: 293)
PGG (SEQ ID NO: 294)
DNIKHVPGGG (SEQ ID NO: 295)
NIKHVPGGG (SEQ ID NO: 296)
IKHVPGGG (SEQ ID NO: 297)
VPGGG (SEQ ID NO: 298)
PGGG (SEQ ID NO: 299)
NIKHVPGGGS (SEQ ID NO: 300)
IKHVPGGGS (SEQ ID NO: 301)
KHVPGGGS (SEQ ID NO: 302)
HVPGGGS (SEQ ID NO: 303)
VPGGGS (SEQ ID NO: 304)
PGGGS (SEQ ID NO: 305)
GGGS (SEQ ID NO: 306)
IKHVPGGGSV (SEQ ID NO: 307)
KHVPGGGSV (SEQ ID NO: 308)
HVPGGGSV (SEQ ID NO: 309)
VPGGGSV (SEQ ID NO: 310)
PGGGSV (SEQ ID NO: 311)
GGGSV (SEQ ID NO: 312)
KHVPGGGSVQ (SEQ ID NO: 313)
HVPGGGSVQ (SEQ ID NO: 314)
VPGGGSVQ (SEQ ID NO: 315)
PGGGSVQ (SEQ ID NO: 316)
GGGSVQ (SEQ ID NO: 317)
HVPGGGSVQI (SEQ ID NO: 318)
VPGGGSVQI (SEQ ID NO: 319)
PGGGSVQI (SEQ ID NO: 320)
GGSVQIVYKS (SEQ ID NO: 321)
GSVQIVYKS (SEQ ID NO: 322)
SVQIVYKS (SEQ ID NO: 323)
VQIVYKS (SEQ ID NO: 324)
QIVYKS (SEQ ID NO: 325)
GSVQIVYKSV (SEQ ID NO: 326)
SVQIVYKSV (SEQ ID NO: 327)
VQIVYKSV (SEQ ID NO: 328)
IVYKSV (SEQ ID NO: 329)
VYKSV (SEQ ID NO: 330)
YKSV (SEQ ID NO: 331)
KSV (SEQ ID NO: 332)
SVQIVYKSVD (SEQ ID NO: 333)
VQIVYKSVD (SEQ ID NO: 334)
QIVYKSVD (SEQ ID NO: 335)
IVYKSVD (SEQ ID NO: 336)
VYKSVD (SEQ ID NO: 337)
YKSVD (SEQ ID NO: 338)
KSVD (SEQ ID NO: 339)
SVD (SEQ ID NO: 340)
VQIVYKSVDL (SEQ ID NO: 341)
QIVYKSVDL (SEQ ID NO: 342)
IVYKSVDL (SEQ ID NO: 343)
VYKSVDL (SEQ ID NO: 344)
YKSVDL (SEQ ID NO: 345)
KSVDL (SEQ ID NO: 346)
SVDL (SEQ ID NO: 347)
QIVYKSVDLS (SEQ ID NO: 348)
IVYKSVDLS (SEQ ID NO: 349)
VYKSVDLS (SEQ ID NO: 350)
YKSVDLS (SEQ ID NO: 351)
KSVDLS (SEQ ID NO: 352)
SVDLS (SEQ ID NO: 353)
IVYKSVDLSK (SEQ ID NO: 354)
VYKSVDLSK (SEQ ID NO: 355)
YKSVDLSK (SEQ ID NO: 356)
KSVDLSK (SEQ ID NO: 357)
SVDLSK (SEQ ID NO: 358)
VYKSVDLSKV (SEQ ID NO: 359)
YKSVDLSKV (SEQ ID NO: 360)
KSVDLSKV (SEQ ID NO: 361)
SVDLSKV (SEQ ID NO: 362)
YKSVDLSKVT (SEQ ID NO: 363)
KSVDLSKVT (SEQ ID NO: 364)
SVDLSKVT (SEQ ID NO: 365)
HGAEIVYKSV (SEQ ID NO: 366)
GAEIVYKSV (SEQ ID NO: 367)
AEIVYKSV (SEQ ID NO: 368)
GAEIVYKSVV (SEQ ID NO: 369)
AEIVYKSVV (SEQ ID NO: 370)
EIVYKSVV (SEQ ID NO: 371)
IVYKSVV (SEQ ID NO: 372)
VYKSVV (SEQ ID NO: 373)
YKSVV (SEQ ID NO: 374)
KSVV (SEQ ID NO: 375)
SVV (SEQ ID NO: 376)
AEIVYKSVVS (SEQ ID NO: 377)
EIVYKSVVS (SEQ ID NO: 378)
IVYKSVVS (SEQ ID NO: 379)
VYKSVVS (SEQ ID NO: 380)
YKSVVS (SEQ ID NO: 381)
KSVVS (SEQ ID NO: 382)
SVVS (SEQ ID NO: 383)
EIVYKSVVSG (SEQ ID NO: 384)
IVYKSVVSG (SEQ ID NO: 385)
VYKSVVSG (SEQ ID NO: 386)
YKSVVSG (SEQ ID NO: 387)
KSVVSG (SEQ ID NO: 388)
SVVSG (SEQ ID NO: 389)
VVSG (SEQ ID NO: 390)
IVYKSVVSGD (SEQ ID NO: 391)
VYKSVVSGD (SEQ ID NO: 392)
YKSVVSGD (SEQ ID NO: 393)
KSVVSGD (SEQ ID NO: 394)
SVVSGD (SEQ ID NO: 395)
VYKSVVSGDT (SEQ ID NO: 396)
YKSVVSGDT (SEQ ID NO: 397)
KSVVSGDT (SEQ ID NO: 398)
SVVSGDT (SEQ ID NO: 399)
VVSGDT (SEQ ID NO: 400)
YKSVVSGDTS (SEQ ID NO: 401)
KSVVSGDTS (SEQ ID NO: 402)
SVVSGDTS (SEQ ID NO: 403)
KSVVSGDTSP (SEQ ID NO: 404)
SVVSGDTSPR (SEQ ID NO: 405)
VVSGDTSPR (SEQ ID NO: 406)
DHGAEIVYKP (SEQ ID NO: 407)
HGAEIVYKP (SEQ ID NO: 408)
GAEIVYKP (SEQ ID NO: 409)
AEIVYKP (SEQ ID NO: 410)
EIVYKP (SEQ ID NO: 411)
HGAEIVYKPV (SEQ ID NO: 412)
GAEIVYKPV (SEQ ID NO: 413)
AEIVYKPV (SEQ ID NO: 414)
GAEIVYKPVV (SEQ ID NO: 415)
AEIVYKPVV (SEQ ID NO: 416)
EIVYKPVV (SEQ ID NO: 417)
IVYKPVV (SEQ ID NO: 418)
VYKPVV (SEQ ID NO: 419)
YKPVV (SEQ ID NO: 420)
KPVV (SEQ ID NO: 421)
AEIVYKPVVS (SEQ ID NO: 422)
EIVYKPVVS (SEQ ID NO: 423)
IVYKPVVS (SEQ ID NO: 424)
VYKPVVS (SEQ ID NO: 425)
YKPVVS (SEQ ID NO: 426)
KPVVS (SEQ ID NO: 427)
PVVS (SEQ ID NO: 428)
EIVYKPVVSG (SEQ ID NO: 429)
IVYKPVVSG (SEQ ID NO: 430)
VYKPVVSG (SEQ ID NO: 431)
YKPVVSG (SEQ ID NO: 432)
KPVVSG (SEQ ID NO: 433)
VVSG (SEQ ID NO: 434)
IVYKPVVSGD (SEQ ID NO: 435)
VYKPVVSGD (SEQ ID NO: 436)
YKPVVSGD (SEQ ID NO: 437)
KPVVSGD (SEQ ID NO: 438)
VYKPVVSGDT (SEQ ID NO: 439)
YKPVVSGDT (SEQ ID NO: 440)
KPVVSGDT (SEQ ID NO: 441)
YKPVVSGDTS (SEQ ID NO: 442)
KPVVSGDTSP (SEQ ID NO: 443)
CNIK (SEQ ID NO: 444)
CNIKH (SEQ ID NO: 445)
CNIKHV (SEQ ID NO: 446)
CNIKHVPGG (SEQ ID NO: 447)
CNIKHVPGGG (SEQ ID NO: 448)
EAAGHVTQC (SEQ ID NO: 449)
EAAGHVTQAR (SEQ ID NO: 450)
AAGHVTQAC (SEQ ID NO: 451)
AGHVTQARC (SEQ ID NO: 452)
AGHVTQAR (SEQ ID NO: 453)
GYTMHQD (SEQ ID NO: 454)
QGGYTMHC (SEQ ID NO: 455)
QGGYTMHQD (SEQ ID NO: 456)
GGYTMHQC (SEQ ID NO: 457)
ENLKHQPGGG (SEQ ID NO: 458)
NLKHQPGGG (SEQ ID NO: 459)
LKHQPGGG (SEQ ID NO: 460)
KHQPGGG (SEQ ID NO: 461)
QPGGG (SEQ ID NO: 462)
TENLKHQPGG (SEQ ID NO: 463)
ENIKHQPGG (SEQ ID NO: 464)
NLKHQPGG (SEQ ID NO: 465)
LKHQPGG (SEQ ID NO: 466)
KHQPGG (SEQ ID NO: 467)
QPGG (SEQ ID NO: 468)
TENLKHQPG (SEQ ID NO: 469)
ENLKHQPG (SEQ ID NO: 470)
NLKHQPG (SEQ ID NO: 471)
LKHQPG (SEQ ID NO: 472)
KHQPG (SEQ ID NO: 473)
HQPG (SEQ ID NO: 474)
QPG (SEQ ID NO: 475)
TENLKHQP (SEQ ID NO: 476)
ENLKHQP (SEQ ID NO: 477)
NLKHQP (SEQ ID NO: 478)
LKHQP (SEQ ID NO: 479)
KHQP (SEQ ID NO: 480)
HQP (SEQ ID NO: 481)
TENLKHQ (SEQ ID NO: 482)
ENLKHQ (SEQ ID NO: 483)
NLKHQ (SEQ ID NO: 484)
LKHQ (SEQ ID NO: 485)
KHQ (SEQ ID NO: 486)
TENLKH (SEQ ID NO: 487)
ENLKH (SEQ ID NO: 488)
NLKH (SEQ ID NO: 489)
LKH (SEQ ID NO: 490)
TENLK (SEQ ID NO: 491)
ENLK (SEQ ID NO: 492)
NLK (SEQ ID NO: 493)
TENL (SEQ ID NO: 494)
ENL (SEQ ID NO: 495)
TEN (SEQ ID NO: 496)
KDNI (SEQ ID NO: 497)
DNI (SEQ ID NO: 498)
KDN (SEQ ID NO: 499)
IKHVGGG (SEQ ID NO: 500)
IKHVGG (SEQ ID NO: 501)
IKHVG (SEQ ID NO: 502)
KHVGGG (SEQ ID NO: 503)
KHVGG (SEQ ID NO: 504)
KHVG (SEQ ID NO: 505)
GNIHHKPGGG (SEQ ID NO: 506)
NIHHKPGGG (SEQ ID NO: 507)
IHHKPGGG (SEQ ID NO: 508)
HHKPGGG (SEQ ID NO: 509)
KPGGG (SEQ ID NO: 510)
LGNIHHKPGG (SEQ ID NO: 511)
GNIHHKPGG (SEQ ID NO: 512)
NIHHKPGG (SEQ ID NO: 513)
IHHKPGG (SEQ ID NO: 514)
HHKPGG (SEQ ID NO: 515)
KPGG (SEQ ID NO: 516)
LGNIHHKPG (SEQ ID NO: 517)
GNIHHKPG (SEQ ID NO: 518)
NIHHKPG (SEQ ID NO: 519)
IHHKPG (SEQ ID NO: 520)
HHKPG (SEQ ID NO: 521)
HKPG (SEQ ID NO: 522)
KPG (SEQ ID NO: 523)
LGNIHHKP (SEQ ID NO: 524)
GNIHHKP (SEQ ID NO: 525)
NIHHKP (SEQ ID NO: 526)
IHHKP (SEQ ID NO: 527)
HHKP (SEQ ID NO: 528)
HKP (SEQ ID NO: 529)
LGNIHHK (SEQ ID NO: 530)
GNIHHK (SEQ ID NO: 531)
NIHHK (SEQ ID NO: 532)
IHHK (SEQ ID NO: 533)
HHK (SEQ ID NO: 534)
LGNIHH (SEQ ID NO: 535)
GNIHH (SEQ ID NO: 536)
NIHH (SEQ ID NO: 537)
IHH (SEQ ID NO: 538)
LGNIH (SEQ ID NO: 539)
GNIH (SEQ ID NO: 540)
NIH (SEQ ID NO: 541)
LGNI (SEQ ID NO: 542)
GNI (SEQ ID NO: 543)
LGN (SEQ ID NO: 544)
DNITHVPGGG (SEQ ID NO: 545)
NITHVPGGG (SEQ ID NO: 546)
ITHVPGGG (SEQ ID NO: 547)
THVPGGG (SEQ ID NO: 548)
LDNITHVPGG (SEQ ID NO: 549)
DNITHVPGG (SEQ ID NO: 550)
NITHVPGG (SEQ ID NO: 551)
ITHVPGG (SEQ ID NO: 552)
THVPGG (SEQ ID NO: 553)
LDNITHVPG (SEQ ID NO: 554)
DNITHVPG (SEQ ID NO: 555)
NITHVPG (SEQ ID NO: 556)
ITHVPG (SEQ ID NO: 557)
THVPG (SEQ ID NO: 558)
HVPG (SEQ ID NO: 559)
VPG (SEQ ID NO: 560)
LDNITHVP (SEQ ID NO: 561)
DNITHVP (SEQ ID NO: 562)
NITHVP (SEQ ID NO: 563)
ITHVP (SEQ ID NO: 564)
THVP (SEQ ID NO: 565)
LDNITHV (SEQ ID NO: 566)
DNITHV (SEQ ID NO: 567)
NITHV (SEQ ID NO: 568)
ITHV (SEQ ID NO: 569)
THV (SEQ ID NO: 570)
LDNITH (SEQ ID NO: 571)
DNITH (SEQ ID NO: 572)
NITH (SEQ ID NO: 573)
ITH (SEQ ID NO: 574)
LDNIT (SEQ ID NO: 575)
DNIT (SEQ ID NO: 576)
NIT (SEQ ID NO: 577)
LDNI (SEQ ID NO: 578)
LDN (SEQ ID NO: 579)
KNVKSKIGST (SEQ ID NO: 580)
NVKSKIGST (SEQ ID NO: 581)
VKSKIGST (SEQ ID NO: 582)
KSKIGST (SEQ ID NO: 583)
SKIGST (SEQ ID NO: 584)
KIGST (SEQ ID NO: 585)
IGST (SEQ ID NO: 586)
GST (SEQ ID NO: 587)
NVKSKIGSTE (SEQ ID NO: 588)
VKSKIGSTE (SEQ ID NO: 589)
KSKIGSTE (SEQ ID NO: 590)
SKIGSTE (SEQ ID NO: 591)
KIGSTE (SEQ ID NO: 592)
IGSTE (SEQ ID NO: 593)
GSTE (SEQ ID NO: 594)
STE (SEQ ID NO: 595)
VKSKIGSTEN (SEQ ID NO: 596)
KSKIGSTEN (SEQ ID NO: 597)
SKIGSTEN (SEQ ID NO: 598)
KIGSTEN (SEQ ID NO: 599)
IGSTEN (SEQ ID NO: 600)
GSTEN (SEQ ID NO: 601)
STEN (SEQ ID NO: 602)
KSKIGSTENL (SEQ ID NO: 603)
SKIGSTENL (SEQ ID NO: 604)
KIGSTENL (SEQ ID NO: 605)
IGSTENL (SEQ ID NO: 606)
GSTENL (SEQ ID NO: 607)
STENL (SEQ ID NO: 608)
SKIGSTENLK (SEQ ID NO: 609)
KIGSTENLK (SEQ ID NO: 610)
IGSTENLK (SEQ ID NO: 611)
GSTENLK (SEQ ID NO: 612)
STENLK (SEQ ID NO: 613)
KIGSTENLKH (SEQ ID NO: 614)
IGSTENLKH (SEQ ID NO: 615)
GSTENLKH (SEQ ID NO: 616)
STENLKH (SEQ ID NO: 617)
IGSTENLKHQ (SEQ ID NO: 618)
GSTENLKHQ (SEQ ID NO: 619)
STENLKHQ (SEQ ID NO: 620)
GSTENLKHQP (SEQ ID NO: 621)
STENLKHQP (SEQ ID NO: 622)
STENLKHQPG (SEQ ID NO: 623)
SNVQSKCGSK (SEQ ID NO: 624)
NVQSKCGSK (SEQ ID NO: 625)
VQSKCGSK (SEQ ID NO: 626)
QSKCGSK (SEQ ID NO: 627)
SKCGSK (SEQ ID NO: 628)
KCGSK (SEQ ID NO: 629)
CGSK (SEQ ID NO: 630)
GSK (SEQ ID NO: 631)
NVQSKCGSKD (SEQ ID NO: 632)
VQSKCGSKD (SEQ ID NO: 633)
QSKCGSKD (SEQ ID NO: 634)
SKCGSKD (SEQ ID NO: 635)
KCGSKD (SEQ ID NO: 636)
CGSKD (SEQ ID NO: 637)
GSKD (SEQ ID NO: 638)
SKD (SEQ ID NO: 639)
VQSKCGSKDN (SEQ ID NO: 640)
QSKCGSKDN (SEQ ID NO: 641)
SKCGSKDN (SEQ ID NO: 642)
KCGSKDN (SEQ ID NO: 643)
CGSKDN (SEQ ID NO: 644)
GSKDN (SEQ ID NO: 645)
SKDN (SEQ ID NO: 646)
QSKCGSKDNI (SEQ ID NO: 647)
SKCGSKDNI (SEQ ID NO: 648)
KCGSKDNI (SEQ ID NO: 649)
CGSKDNI (SEQ ID NO: 650)
GSKDNI (SEQ ID NO: 651)
SKDNI (SEQ ID NO: 652)
GSKDNIKH (SEQ ID NO: 653)
GSKDNIKHV (SEQ ID NO: 654)
GSKDNIKHVP (SEQ ID NO: 655)
SKVTSKCGSL (SEQ ID NO: 656)
KVTSKCGSL (SEQ ID NO: 657)
VTSKCGSL (SEQ ID NO: 658)
TSKCGSL (SEQ ID NO: 659)
SKCGSL (SEQ ID NO: 660)
KCGSL (SEQ ID NO: 661)
CGSL (SEQ ID NO: 662)
GSL (SEQ ID NO: 663)
KVTSKCGSLG (SEQ ID NO: 664)
VTSKCGSLG (SEQ ID NO: 665)
TSKCGSLG (SEQ ID NO: 666)
SKCGSLG (SEQ ID NO: 667)
KCGSLG (SEQ ID NO: 668)
CGSLG (SEQ ID NO: 669)
GSLG (SEQ ID NO: 670)
SLG (SEQ ID NO: 671)
VTSKCGSLGN (SEQ ID NO: 672)
TSKCGSLGN (SEQ ID NO: 673)
SKCGSLGN (SEQ ID NO: 674)
KCGSLGN (SEQ ID NO: 675)
CGSLGN (SEQ ID NO: 676)
GSLGN (SEQ ID NO: 677)
SLGN (SEQ ID NO: 678)
TSKCGSLGNI (SEQ ID NO: 679)
SKCGSLGNI (SEQ ID NO: 680)
KCGSLGNI (SEQ ID NO: 681)
CGSLGNI (SEQ ID NO: 682)
GSLGNI (SEQ ID NO: 683)
SLGNI (SEQ ID NO: 684)
SKCGSLGNIH (SEQ ID NO: 685)
KCGSLGNIH (SEQ ID NO: 686)
CGSLGNIH (SEQ ID NO: 687)
GSLGNIH (SEQ ID NO: 688)
SLGNIH (SEQ ID NO: 689)
KCGSLGNIHH (SEQ ID NO: 690)
CGSLGNIHH (SEQ ID NO: 691)
GSLGNIHH (SEQ ID NO: 692)
SLGNIHH (SEQ ID NO: 693)
CGSLGNIHHK (SEQ ID NO: 694)
GSLGNIHHK (SEQ ID NO: 695)
SLGNIHHK (SEQ ID NO: 696)
GSLGNIHHKP (SEQ ID NO: 697)
SLGNIHHKP (SEQ ID NO: 698)
SLGNIHHKPG (SEQ ID NO: 699)
DRVQSKIGSL (SEQ ID NO: 700)
RVQSKIGSL (SEQ ID NO: 701)
VQSKIGSL (SEQ ID NO: 702)
QSKIGSL (SEQ ID NO: 703)
SKIGSL (SEQ ID NO: 704)
KIGSL (SEQ ID NO: 705)
IGSL (SEQ ID NO: 706)
RVQSKIGSLD (SEQ ID NO: 707)
VQSKIGSLD (SEQ ID NO: 708)
QSKIGSLD (SEQ ID NO: 709)
SKIGSLD (SEQ ID NO: 710)
KIGSLD (SEQ ID NO: 711)
IGSLD (SEQ ID NO: 712)
GSLD (SEQ ID NO: 713)
SLD (SEQ ID NO: 714)
VQSKIGSLDN (SEQ ID NO: 715)
QSKIGSLDN (SEQ ID NO: 716)
SKIGSLDN (SEQ ID NO: 717)
KIGSLDN (SEQ ID NO: 718)
IGSLDN (SEQ ID NO: 719)
GSLDN (SEQ ID NO: 720)
SLDN (SEQ ID NO: 721)
QSKIGSLDNI (SEQ ID NO: 722)
SKIGSLDNI (SEQ ID NO: 723)
KIGSLDNI (SEQ ID NO: 724)
IGSLDNI (SEQ ID NO: 725)
GSLDNI (SEQ ID NO: 726)
SKIGSLDNIT (SEQ ID NO: 727)
KIGSLDNIT (SEQ ID NO: 728)
IGSLDNIT (SEQ ID NO: 729)
GSLDNIT (SEQ ID NO: 730)
SLDNIT (SEQ ID NO: 731)
KIGSLDNITH (SEQ ID NO: 732)
IGSLDNITH (SEQ ID NO: 733)
GSLDNITH (SEQ ID NO: 734)
SLDNITH (SEQ ID NO: 735)
IGSLDNITHV (SEQ ID NO: 736)
GSLDNITHV (SEQ ID NO: 737)
SLDNITHV (SEQ ID NO: 738)
GSLDNITHVP (SEQ ID NO: 739)
SLDNITHVP (SEQ ID NO: 740)
SLDNITHVPG (SEQ ID NO: 741)
Lys Xaa₁ Xaa₂ Ser Xaa₃ Xaa₄ Asn Xaa₅ Xaa₆ His (SEQ ID NO: 742),
wherein
Xaa₁ is I or C;
Xaa₂ is G;
Xaa₃ is T, K or L,
Xaa₄ is E, D or G,
Xaa₅ is L or I,
Xaa₆ is K, H or T.
Arg-Val-Arg-Arg (SEQ ID NO: 743),
Gly-Ala-Gly-Ala (SEQ ID NO: 744),
Ala-Gly-Ala-Gly (SEQ ID NO: 745),
Lys-Gly-Lys-Gly (SEQ ID NO: 746),
Lys Xaa₇ Xaa₇ Ser Xaa₇ Xaa₇ Asn Xaa₇ Xaa₈ His (SEQ ID NO: 747),
wherein
Xaa₇ is any amino acid, and
Xaa₈ is K or 543H.
Xaa₉ Ile Val Tyr Lys Xaa₁₀ (SEQ ID NO: 748),
wherein
Xaa₉ is Gln or Glu, and
Xaa₁₀ is Ser or Pro.
Ser Lys Xaa₁₁ Gly Ser (SEQ ID NO: 749),
wherein
Xaa₁₁ is I or C.
SEQ ID NO: 750 - Tau P10636-1,
MAEPRQEFEV MEDHAGTYGL GDRKDQGGYT MHQDQEGDTD AGLKESPLQT
PTEDGSEEPG SETSDAKSTP TAEDVTAPLV DEGAPGKQAA AQPHTEIPEG
TTAEEAGIGD TPSLEDEAAG HVTQEPESGK VVQEGFLREP GPPGLSHQLM
SGMPGAPLLP EGPREATRQP SGTGPEDTEG GRHAPELLKH QLLGDLHQEG
PPLKGAGGKE RPGSKEEVDE DRDVDESSPQ DSPPSKASPA QDGRPPQTAA
REATSIPGFP AEGAIPLPVD FLSKVSTEIP ASEPDGPSVG RAKGQDAPLE
FTFHVEITPN VQKEQAHSEE HLGRAAFPGA PGEGPEARGP SLGEDTKEAD
LPEPSEKQPA AAPRGKPVSR VPQLKARMVS KSKDGTGSDD KKAKTSTRSS
AKTLKNRPCL SPKHPTPGSS DPLIQPSSPA VCPEPPSSPK YVSSVTSRTG
SSGAKEMKLK GADGKTKIAT PRGAAPPGQK GQANATRIPA KTPPAPKTPP
SSGEPPKSGD RSGYSSPGSP GTPGSRSRTP SLPTPPTREP KKVAVVRTPP
KSPSSAKSRL QTAPVPMPDL KNVKSKIGST ENLKHQPGGG KVQIINKKLD
LSNVQSKCGS KDNIKHVPGG GSVQIVYKPV DLSKVTSKCG SLGNIHHKPG
GGQVEVKSEK LDFKDRVQSK IGSLDNITHV PGGGNKKIET HKLTFRENAK
AKTDHGAEIV YKSPVVSGDT SPRHLSNVSS TGSIDMVDSP QLATLADEVS
ASLAKQGL
MTBR peptide 1 (SEQ ID NO: 751):
QTAPVPMPDLKNVKSKIGSTENLKHQPGGGK
MTBR peptide 2, (SEQ ID NO: 752):
VQIINKKLDLSNVQSKCGSKDNIKHVPGGGS
MTBR peptide 3, (SEQ ID NO: 753)
VQIVYKPVDLSKVTSKCGSLGNIHHKPGGGQ
MTBR peptide 4, (SEQ ID NO: 754)
VEVKSEKLDFKDRVQSKIGSLDNITHVPGGGN
PDLKNVKS (SEQ ID NO: 755)
DLKNVKSK (SEQ ID NO: 756)
LKNVKSKI (SEQ ID NO: 757)
KNVKSKIG (SEQ ID NO: 758)
NVKSKIGS (SEQ ID NO: 759)
LDLSNVQS (SEQ ID NO: 760)
DLSNVQSK (SEQ ID NO: 761)
LSNVQSKC (SEQ ID NO: 762)
SNVQSKCG (SEQ ID NO: 763)
NVQSKCGS (SEQ ID NO: 764)
VDLSKVTS (SEQ ID NO: 765)
DLSKVTSK (SEQ ID NO: 766)
LSKVTSKC (SEQ ID NO: 767)
SKVTSKCG (SEQ ID NO: 768)
KVTSKCGS (SEQ ID NO: 769)
VTSKCGSL (SEQ ID NO: 770)
LDFKDRVQ (SEQ ID NO: 771)
DFKDRVQS (SEQ ID NO: 772)
FKDRVQSK (SEQ ID NO: 773)
KDRVQSKI (SEQ ID NO: 774)
DRVQSKIG (SEQ ID NO: 775)
RVQSKIGS (SEQ ID NO: 776)
VKSKIGSTEGGC (SEQ ID NO: 777)
KSKIGSTEGGC (SEQ ID NO: 778)
SKIGSTENGGC (SEQ ID NO: 779)
KIGSTENLGGC (SEQ ID NO: 780)
IGSTENLKGGC (SEQ ID NO: 781)
GSTENLKHGGC (SEQ ID NO: 782)
STENLKHQGGC (SEQ ID NO: 783)
TENLKHQPGGC (SEQ ID NO: 784)
ENLKHQPGGGC (SEQ ID NO: 785)
VKSKIGSTGGC (SEQ ID NO: 786)
PDLKNVKSGGC (SEQ ID NO: 787)
DLKNVKSKGGC (SEQ ID NO: 788)
LKNVKSKIGGC (SEQ ID NO: 789)
KNVKSKIGGGC (SEQ ID NO: 790)
NVKSKIGSGGC (SEQ ID NO: 791)
NLKHQPGGGGC (SEQ ID NO: 792)
LKHQPGGGGGC (SEQ ID NO: 793)
LDLSNVQSGGC (SEQ ID NO: 794)
DLSNVQSKGGC (SEQ ID NO: 795)
LSNVQSKCGGC (SEQ ID NO: 796)
SNVQSKCGGGC (SEQ ID NO: 797)
NVQSKCGSGGC (SEQ ID NO: 798)
VQSKCGSKGGC (SEQ ID NO: 799)
QSKCGSKDGGC (SEQ ID NO: 800)
SKCGSKDNGGC (SEQ ID NO: 801)
KCGSKDNIGGC (SEQ ID NO: 802)
CGSKDNIKGGC (SEQ ID NO: 803)
GSKDNIKHGGC (SEQ ID NO: 804)
SKDNIKHVGGC (SEQ ID NO: 805)
KDNIKHVPGGC (SEQ ID NO: 806)
DNIKHVPGGGC (SEQ ID NO: 807)
NIKHVPGGGGC (SEQ ID NO: 808)
IKHVPGGGGGC (SEQ ID NO: 809)
VDLSKVTSGGC (SEQ ID NO: 810)
DLSKVTSKGGC (SEQ ID NO: 811)
LSKVTSKCGGC (SEQ ID NO: 812)
SKVTSKCGGGC (SEQ ID NO: 813)
KVTSKCGSGGC (SEQ ID NO: 814)
VTSKCGSLGGC (SEQ ID NO: 815)
TSKCGSLGGGC (SEQ ID NO: 816)
SKCGSLGNGGC (SEQ ID NO: 817)
KCGSLGNIGGC (SEQ ID NO: 818)
COSLGNIHGGC (SEQ ID NO: 819)
GSLGNIHHGGC (SEQ ID NO: 820)
SLGNIHHKGGC (SEQ ID NO: 821)
LGNIHHKPGGC (SEQ ID NO: 822)
GNIHHKPGGGC (SEQ ID NO: 823)
NIHHKPGGGGC (SEQ ID NO: 824)
IHHKPGGGGGC (SEQ ID NO: 825)
LDFKDRVQGGC (SEQ ID NO: 826)
DFKDRVQSGGC (SEQ ID NO: 827)
FKDRVQSKGGC (SEQ ID NO: 828)
KDRVQSKIGGC (SEQ ID NO: 829)
DRVQSKIGGGC (SEQ ID NO: 830)
RVQSKIGSGGC (SEQ ID NO: 831)
VQSKIGSLGGC (SEQ ID NO: 832)
QSKIGSLDGGC (SEQ ID NO: 833)
SKIGSLDNGGC (SEQ ID NO: 834)
KIGSLDNIGGC (SEQ ID NO: 835)
IGSLDNITGGC (SEQ ID NO: 836)
GSLDNITHGGC (SEQ ID NO: 837)
SLDNITHVGGC (SEQ ID NO: 838)
LDNITHVPGGC (SEQ ID NO: 839)
DNITHVPGGGC (SEQ ID NO: 840)
NITHVPGGGGC (SEQ ID NO: 841)
ITHVPGGGGGC (SEQ ID NO: 842)
VKSKIGSTEGGGC (SEQ ID NO: 843)
KSKIGSTEGGGC (SEQ ID NO: 844)
SKIGSTENGGGC (SEQ ID NO: 845)
KIGSTENLGGGC (SEQ ID NO: 846)
IGSTENLKGGGC (SEQ ID NO: 847)
GSTENLKHGGGC (SEQ ID NO: 848)
STENLKHQGGGG (SEQ ID NO: 849)
TENLKHQPGGGC (SEQ ID NO: 850)
VKSKIGSTGGGC (SEQ ID NO: 851)
PDLKNVKSGGGC (SEQ ID NO: 852)
DLKNVKSKGGGC (SEQ ID NO: 853)
LKNVKSKIGGGG (SEQ ID NO: 854)
KNVKSKIGGGGC (SEQ ID NO: 855)
NVKSKIGSGGGC (SEQ ID NO: 856)
ENLKHQPGGGGC (SEQ ID NO: 837)
NLKHQPGGGGGC (SEQ ID NO: 858)
LKHQPGGGGGGC (SEQ ID NO: 859)
LDLSNVQSGGGC (SEQ ID NO: 860)
DLSNVQSKGGGC (SEQ ID NO: 861)
LSNVQSKCOGGC (SEQ ID NO: 862)
SNVQSKCGGGGC (SEQ ID NO: 863)
NVQSKCGSGGGC (SEQ ID NO: 864)
VQSKCGSKGGGC (SEQ ID NO: 865)
QSKCGSKDGGGC (SEQ ID NO: 866)
SKCGSKDNGGGC (SEQ ID NO: 867)
KCGSKDNIGGGC (SEQ ID NO: 868)
CGSKDNIKGGGC (SEQ ID NO: 869)
GSKDNIKHGGGC (SEQ ID NO: 870)
SKDNIKHVGGGC (SEQ ID NO: 871)
KDNIKHVPGGGC (SEQ ID NO: 872)
DNIKHVPGGGGC (SEQ ID NO: 873)
NIKHVPGGGGGC (SEQ ID NO: 874)
IKHVPGGGGGGC (SEQ ID NO: 875)
VDLSKVTSGGGC (SEQ ID NO: 876)
DLSKVTSKGGGC (SEQ ID NO: 877)
LSKVTSKCGGGC (SEQ ID NO: 878)
SKVTSKCGGGGC (SEQ ID NO: 879)
KVTSKCGSGGGC (SEQ ID NO: 880)
VTSKCGSLGGGC (SEQ ID NO: 881)
TSKCGSLGGGGC (SEQ ID NO: 882)
SKCGSLGNGGGC (SEQ ID NO: 883)
KCGSLGNIGGGC (SEQ ID NO: 884)
CGSLGNIHGGGC (SEQ ID NO: 885)
GSLGNIHHGGGC (SEQ ID NO: 886)
SLGNIHHKGGGC (SEQ ID NO: 887)
LGNIHHKPGGGC (SEQ ID NO: 888)
GNIHHKPGGGGC (SEQ ID NO: 889)
NIHHKPGGGGGC (SEQ ID NO: 890)
IHHKPGGGGGGC (SEQ ID NO: 891)
LDFKDRVQGGGG (SEQ ID NO: 892)
DFKDRVQSGGGC (SEQ ID NO: 893)
FKDRVQSKGGGG (SEQ ID NO: 894)
KDRVQSKIGGGC (SEQ ID NO: 895)
DRVQSKIGGGGC (SEQ ID NO: 896)
RVQSKIGSGGGC (SEQ ID NO: 897)
VQSKIGSLGGGC (SEQ ID NO: 898)
QSKIGSLDGGGC (SEQ ID NO: 899)
SKIGSLDNGGGC (SEQ ID NO: 900)
KIGSLDNIGGGC (SEQ ID NO: 901)
IGSLDNITGGGC (SEQ ID NO: 902)
GSLDNITHGGGC (SEQ ID NO: 903)
SLDNITHVGGGC (SEQ ID NO: 904)
LDNITHVPGGGC (SEQ ID NO: 905)
DNITHVPGGGGC (SEQ ID NO: 906)
NITHVPGGGGGC (SEQ ID NO: 907)
ITHVPGGGGGGC (SEQ ID NO: 908)
SKIGSTENLKH (SEQ ID NO: 909)
SKIGSTENIKH (SEQ ID NO: 910)
SKIGSKDNLKH (SEQ ID NO: 911)
SKIGSKENIKH (SEQ ID NO: 912)
SKIGSLENLKH (SEQ ID NO: 913)
SKIGSLENIKH (SEQ ID NO: 914)
SKIGSTDNLKH (SEQ ID NO: 915)
SKIGSTDNIKH (SEQ ID NO: 916)
SKIGSKDNLKH (SEQ ID NO: 917)
SKIGSKDNIKH (SEQ ID NO: 918)
SKIGSLDNLKH (SEQ ID NO: 919)
SKIGSLDNIKH (SEQ ID NO: 920)
SKIGSTGNLKH (SEQ ID NO: 921)
SKIGSTGNIKH (SEQ ID NO: 922)
SKIGSKGNLKH (SEQ ID NO: 923)
SKIGSKGNIKH (SEQ ID NO: 924)
SKIGSLGNLKH (SEQ ID NO: 925)
SKIGSLGNIKH (SEQ ID NO: 926)
SKIGSTENLKHGGC (SEQ ID NO: 927)
SKIGSTENIKHGGC (SEQ ID NO: 928)
SKIGSKDNLKHGGC (SEQ ID NO: 929)
SKIGSKENIKHGGC (SEQ ID NO: 930)
SKIGSLENLKHGGC (SEQ ID NO 931)
SKIGSLENIKHGGC (SEQ ID NO: 932)
SKIGSTDNLKHGGC (SEQ ID NO: 933)
SKIGSTDNIKHGGC (SEQ ID NO: 934)
SKIGSKDNLKHGGC (SEQ ID NO: 935)
SKIGSKDNIKHGGC (SEQ ID NO: 936)
SKIGSLDNLKHGGC (SEQ ID NO: 937)
SKIGSLDNIKHGGC (SEQ ID NO: 938)
SKIGSTGNLKHGGC (SEQ ID NO: 939)
SKIGSTGNIKHGGC (SEQ ID NO: 940)
SKIGSKGNLKHGGC (SEQ ID NO: 941)
SKIGSKGNIKHGGC (SEQ ID NO: 942)
SKIGSLGNLKHGGC (SEQ ID NO: 943)
SKIGSLGNIKHGGC (SEQ ID NO: 944)
SKIGSTENLKHGGGC (SEQ ID NO: 945)
SKIGSTENIKHGGGC (SEQ ID NO: 946)
SKIGSKDNLKHGGGC (SEQ ID NO: 947)
SKIGSKENIKHGGGC (SEQ ID NO: 948)
SKIGSLENLKHGGGC (SEQ ID NO: 949)
SKIGSLENIKHGGGC (SEQ ID NO: 950)
SKIGSTDNLKHGGGC (SEQ ID NO: 951)
SKIGSTDNIKHGGGC (SEQ ID NO: 952)
SKIGSKDNLKHGGGC (SEQ ID NO: 953)
SKIGSKDNIKHGGGC (SEQ ID NO: 954)
SKIGSLDNLKHGGGC (SEQ ID NO: 955)
SKIGSLDNIKHGGGC (SEQ ID NO: 956)
SKIGSTGNLKHGGGC (SEQ ID NO: 957)
SKIGSTGNIKHGGGC (SEQ ID NO: 958)
SKIGSKGNLKHGGGC (SEQ ID NO: 959)
SKIGSKGNIKHGGGC (SEQ ID NO: 960)
SKIGSLGNLKHGGGC (SEQ ID NO: 961)
SKIGSLGNIKHGGGC (SEQ ID NO: 962)
CGGSKIGSTDNIKH (SEQ ID NO: 963)
CGGSKIGSKDNIKH (SEQ ID NO: 964)
CGGSKIGSLDNIKH (SEQ ID NO: 965)
CGGGSKIGSTDNIKH (SEQ ID NO: 966)
CGGGSKIGSKDNIKH (SEQ ID NO: 967)
CGGGSKIGSLDNIKH (SEQ ID NO: 968)
GGGS (SEQ ID NO: 969)
GGGGS (SEQ ID NO: 970).

What is claimed is:

1. A peptide comprising 3-13 amino acids from residues 244-400 of SEQ ID NO:01 or from residues 1-150 of SEQ ID NO:750.

2. The peptide of claim 1, wherein the peptide is from the microtubule binding region (MTBR) of tau (residues 244-372 of SEQ ID NO:01).

3. The peptide of claim 1, wherein the peptide comprises an amino acid sequence selected from the group consisting of any one of SEQ ID NO:02 to SEQ ID NO:19, SEQ ID NO:25 to SEQ ID NO:320, SEQ ID NO:411, SEQ ID NO:454, SEQ ID NO:456, SEQ ID NO:458 to SEQ ID NO:742, SEQ ID NO:747 to SEQ ID NO:749, or SEQ ID NO:755 to SEQ ID NO:776.

4. The peptide of claim 3, wherein the peptide comprises an amino acid sequence selected from the group consisting of any one of SEQ ID NO:02 to SEQ ID NO:19, SEQ ID NO:28 to SEQ ID NO:102, SEQ ID NO:185 to SEQ ID NO:320, SEQ ID NO:458 to SEQ ID NO:742, or SEQ ID NO:747 to SEQ ID NO:749.

5. The peptide of any one of claims 1-4, wherein the peptide further comprises a C-terminal cysteine (—C) or an N-terminal cysteine (C—).

6. The peptide of any one of claims 1-4, wherein the peptide further comprises a C-terminal -GGC or -GGGC.

7. The peptide of any one of claims 1-4, wherein the peptide further comprises a N-terminal CGG or CGGG-.

8. The peptide of any one of claims 1-7, wherein the peptide comprises 7-13 amino acids from residues 244-400 of SEQ ID NO:01 or from residues 1-150 of SEQ ID NO:750.

9. The peptide of any one of claims 1-7, wherein the peptide comprises 8 amino acids from residues 244-400 of SEQ ID NO:01.

10. The peptide of any one of claims 1-7, wherein the peptide comprises 8 amino acids from residues 1-150 of SEQ ID NO:750.

11. The peptide of any one of claims 1-7, wherein the peptide comprises an amino acid sequence selected from the group consisting of any one of SEQ ID NO:777 to SEQ ID NO:908.

12. A peptide comprising an amino acid sequence selected from the group consisting of any one of SEQ ID NO:20 to SEQ ID NO:24, SEQ ID NO:312 to SEQ ID NO:457, each optionally further comprising a C-terminal cysteine.

13. The peptide of any one of claims 1-7, wherein the peptide comprises an amino acid sequence selected from the group consisting of
(SEQ ID NO: 02)
QIVYKPV,
(SEQ ID NO: 03)
QIVYKP,
(SEQ ID NO: 04)
NIKHVP,
(SEQ ID NO: 05)
NIKHVPG,
(SEQ ID NO: 06)
HVPGGG,
(SEQ ID NO: 07)
HVPGG,
(SEQ ID NO: 08)
HKPGGG,
(SEQ ID NO: 09)
HKPGG,
(SEQ ID NO: 10)
KHVPGGG,
(SEQ ID NO: 11)
KHVPGG,
(SEQ ID NO: 12)
HQPGGG,
(SEQ ID NO: 13)
HQPGG,
(SEQ ID NO: 14)
VQIINK,
(SEQ ID NO: 15)
VQIINKK,
(SEQ ID NO: 16)
VQIINKKL,
(SEQ ID NO: 17)
QIINK,
(SEQ ID NO: 18)
QIINKK,
(SEQ ID NO: 19)
QIINKKL,
(SEQ ID NO: 25)
EIVYKSP,
(SEQ ID NO: 26)
IVYKSPV,
(SEQ ID NO: 27)
IVYK,
(SEQ ID NO: 325)
QIVYKS,
(SEQ ID NO: 131)
EIVYKS,
(SEQ ID NO: 411)
EIVYKP,
(SEQ ID NO: 454)
GYTMHQD,
(SEQ ID NO: 456)
QGGYTMHQD,
(SEQ ID NO: 589)
VKSKIGSTE,
(SEQ ID NO: 590)
KSKIGSTE,
(SEQ ID NO: 598)
SKIGSTEN,
(SEQ ID NO: 605)
KIGSTENL,
(SEQ ID NO: 611)
IGSTENLK,
(SEQ ID NO: 616)
GSTENLKH,
(SEQ ID NO: 620)
STENLKHQ,
(SEQ ID NO: 476)
TENLKHQP,
(SEQ ID NO: 470)
ENLKHQPG,
(SEQ ID NO: 582)
VKSKIGST,
(SEQ ID NO: 755)
PDLKNVKS,
(SEQ ID NO: 756)
DLKNVKSK,
(SEQ ID NO: 757)
LKNVKSKI,
(SEQ ID NO: 758)
KNVKSKIG,
(SEQ ID NO: 759)
NVKSKIGS,
(SEQ ID NO: 465)
NLKHQPGG,
(SEQ ID NO: 460)
LKHQPGGG,
(SEQ ID NO: 760)
LDLSNVQS,
(SEQ ID NO: 761)
DLSNVQSK,
(SEQ ID NO: 762)
LSNVQSKC,
(SEQ ID NO: 763)
SNVQSKCG,
(SEQ ID NO: 764)
NVQSKOGS,
(SEQ ID NO: 626)
VQSKCGSK,
(SEQ ID NO: 634)
QSKCGSKD,
(SEQ ID NO: 642)
SKCGSKDN,
(SEQ ID NO: 649)
KCGSKDNI,
(SEQ ID NO: 258)
CGSKDNIK,
(SEQ ID NO: 653)
GSKDNIKH,
(SEQ ID NO: 271)
SKDNIKHV,
(SEQ ID NO: 278)
KDNIKHVP,
(SEQ ID NO: 285)
DNIKHVPG,
(SEQ ID NO: 292)
NIKHVPGG,
(SEQ ID NO: 297)
IKHVPGGG,
(SEQ ID NO: 765)
VDLSKVTS,
(SEQ ID NO: 766)
DLSKVTSK,
(SEQ ID NO: 767)
LSKVTSKC,
(SEQ ID NO: 768)
SKVTSKCG,
(SEQ ID NO: 769)
KVTSKCGS,
(SEQ ID NO: 770)
VTSKCGSL,
(SEQ ID NO: 666)
TSKCGSLG,
(SEQ ID NO: 674)
SKCGSLGN,
(SEQ ID NO: 681)
KCGSLGNI,
(SEQ ID NO: 687)
CGSLGNIH,
(SEQ ID NO: 692)
GSLGNIHH,
(SEQ ID NO: 696)
SLGNIHHK,
(SEQ ID NO: 524)
LGNIHHKP,
(SEQ ID NO: 518)
GNIHHKPG,
(SEQ ID NO: 513)
NIHHKPGG,
(SEQ ID NO: 508)
IHHKPGGG,
(SEQ ID NO: 771)
LDFKDRVQ,
(SEQ ID NO: 772)
DFKDRVQS,
(SEQ ID NO: 773)
FKDRVQSK,
(SEQ ID NO: 774)
KDRVQSKI,
(SEQ ID NO: 775)
DRVQSKIG,
(SEQ ID NO: 776)
RVQSKIGS,
(SEQ ID NO: 702)
VQSKIGSL,
(SEQ ID NO: 709)
QSKIGSLD,
(SEQ ID NO: 717)
SKIGSLDN,
(SEQ ID NO: 724)
KIGSLDNI,
(SEQ ID NO: 729)
IGSLDNIT,
(SEQ ID NO: 734)
GSLDNITH,
(SEQ ID NO: 738)
SLDNITHV,
(SEQ ID NO: 561)
LDNITHVP,
(SEQ ID NO: 555)
DNITHVPG,
(SEQ ID NO: 551)
NITHVPGG,
and
(SEQ ID NO: 547)
ITHVPGGG.

14. The peptide of claim 12, wherein the peptide comprises and amino acid sequence selected from the group consisting of
(SEQ ID NO: 20)
QIVYKSV,
(SEQ ID NO: 21)
EIVYKSV,
(SEQ ID NO: 22)
EIVYKPV,
(SEQ ID NO: 23)
CNIKHVP,
(SEQ ID NO: 24)
CNIKHVPG,
(SEQ ID NO: 449)
EAAGHVTQC,
(SEQ ID NO: 450)
EAAGHVTQAR,
(SEQ ID NO: 451)
AAGHVTQAC,
(SEQ ID NO: 452)
AGHVTQARC,
(SEQ ID NO: 453)
AGHVTQAR,
(SEQ ID NO: 455)
QGGYTMHC,
and
(SEQ ID NO: 457)
GGYTMHQC.

15. The peptide of either one of claim 13 or 14, wherein the peptide further comprises a C-terminal cysteine (—C), -GGC or -GGGC or an N-terminal cysteine (C—), CGG- or CGGG-.

16. The peptide of claim 13, wherein the peptide comprises an amino acid sequence selected from the group consisting of
(SEQ ID NO: 589)
VKSKIGSTE,
(SEQ ID NO: 590)
KSKIGSTE,
(SEQ ID NO: 598)
SKIGSTEN,
(SEQ ID NO: 605)
KIGSTENL,
(SEQ ID NO: 611)
IGSTENLK,
(SEQ ID NO: 616)
GSTENLKH,
(SEQ ID NO: 620)
STENLKHQ,
(SEQ ID NO: 476)
TENLKHQP,
and
(SEQ ID NO: 470)
ENLKHQPG.

17. The peptide of claim 11, wherein the peptide comprises an amino acid sequence selected from the group consisting of
(SEQ ID NO: 777)
VKSKIGSTEGGC,
(SEQ ID NO: 778)
KSKIGSTEGGC,
(SEQ ID NO: 779)
SKIGSTENGGC,
(SEQ ID NO: 780)
KIGSTENLGGC,
(SEQ ID NO: 781)
IGSTENLKGGC,
(SEQ ID NO: 782)
GSTENLKHGGC,
(SEQ ID NO: 783)
STENLKHQGGC,
(SEQ ID NO: 784)
TENLKHQPGGC,
(SEQ ID NO: 785)
ENLKHQPGGGC,
(SEQ ID NO: 786)
VKSKIGSTGGC,
(SEQ ID NO: 787)
PDLKNVKSGGC,
(SEQ ID NO: 788)
DLKNVKSKGGC,
(SEQ ID NO: 789)
LKNVKSKIGGC,
(SEQ ID NO: 790)
KNVKSKIGGGC,
(SEQ ID NO: 791)
NVKSKIGSGGC,
(SEQ ID NO: 792)
NLKHQPGGGGC,
and
(SEQ ID NO: 793)
LKHQPGGGGGC,

18. The peptide of claim 11, wherein the peptide comprises an amino acid sequence selected from the group consisting of
(SEQ ID NO: 794)
LDLSNVQSGGC,
(SEQ ID NO: 795)
DLSNVQSKGGC,
(SEQ ID NO: 796)
LSNVQSKCGGC,
(SEQ ID NO: 797)
SNVQSKCGGGC,
(SEQ ID NO: 798)
NVQSKOGSGGC,
(SEQ ID NO: 799)
VQSKCGSKGGC,
(SEQ ID NO: 800)
QSKCGSKDGGC,
(SEQ ID NO: 801)
SKCGSKDNGGC,
(SEQ ID NO: 802)
KCGSKDNIGGC,
(SEQ ID NO: 803)
CGSKDNIKGGC,
(SEQ ID NO: 804)
GSKDNIKHGGC,
(SEQ ID NO: 805)
SKDNIKHVGGC,
(SEQ ID NO: 806)
KDNIKHVPGGC,
(SEQ ID NO: 807)
DNIKHVPGGGC,
(SEQ ID NO: 808)
NIKHVPGGGGC,
and
(SEQ ID NO: 809)
IKHVPGGGGGC,

19. The peptide of claim 11, wherein the peptide comprises an amino acid sequence selected from the group consisting of
(SEQ ID NO: 810)
VDLSKVISGGC,
(SEQ ID NO: 811)
DLSKVTSKGGC,
(SEQ ID NO: 812)
LSKVTSKCGGC,
(SEQ ID NO: 813)
SKVTSKCGGGC,
(SEQ ID NO: 814)
KVTSKCGSGGC,
(SEQ ID NO: 815)
VTSKCGSLGGC,
(SEQ ID NO: 816)
TSKCGSLGGGC,
(SEQ ID NO: 817)
SKCGSLGNGGC,
(SEQ ID NO: 818)
KCGSLGNIGGC,
(SEQ ID NO: 819)
CGSLGNIHGGC,
(SEQ ID NO: 820)
GSLGNIHHGGC,
(SEQ ID NO: 821)
SLGNIHHKGGC,
(SEQ ID NO: 822)
LGNIHHKPGGC,
(SEQ ID NO: 823)
GNIHHKPGGGC,
(SEQ ID NO: 824)
NIHHKPGGGGC,
and
(SEQ ID NO: 825)
IHHKPGGGGGC,

20. The peptide of claim 11, wherein the peptide comprises an amino acid sequence selected from the group consisting of
(SEQ ID NO: 826)
LDFKDRVQGGC,
(SEQ ID NO: 827)
DFKDRVQSGGC,
(SEQ ID NO: 828)
FKDRVQSKGGC,
(SEQ ID NO: 829)
KDRVQSKIGGC,
(SEQ ID NO: 830)
DRVQSKIGGGC,
(SEQ ID NO: 831)
RVQSKIGSGGC,
(SEQ ID NO: 832)
VQSKIGSLGGC,
(SEQ ID NO: 833)
QSKIGSLDGGC,
(SEQ ID NO: 834)
SKIGSLDNGGC,
(SEQ ID NO: 835)
KIGSLDNIGGC,
(SEQ ID NO: 836)
IGSLDNITGGC,
(SEQ ID NO: 837)
GSLDNITHGGC,
(SEQ ID NO: 838)
SLDNITHVGGC,
(SEQ ID NO: 839)
LDNITHVPGGC,
(SEQ ID NO: 840)
DNITHVPGGGC,
(SEQ ID NO: 841)
NITHVPGGGGC,
and
(SEQ ID NO: 842)
ITHVPGGGGGC.

21. The peptide of claim 17, wherein the peptide comprises an amino acid sequence selected from the group consisting of
(SEQ ID NO: 777)
VKSKIGSTEGGC,
(SEQ ID NO: 778)
KSKIGSTEGGC,
(SEQ ID NO: 779)
SKIGSTENGGC,
(SEQ ID NO: 780)
KIGSTENLGGC,
(SEQ ID NO: 781)
IGSTENLKGGC,
(SEQ ID NO: 782)
GSTENLKHGGC,
(SEQ ID NO: 783)
STENLKHQGGC,
(SEQ ID NO: 784)
TENLKHQPGGC,
and
(SEQ ID NO: 785)
ENLKHQPGGGC.

22. The peptide of claim 18, wherein the peptide comprises an amino acid sequence selected from the group consisting of
(SEQ ID NO: 799)
VQSKCGSKGGC,
(SEQ ID NO: 800)
QSKCGSKDGGC,
(SEQ ID NO: 801)
SKCGSKDNGGC,
(SEQ ID NO: 802)
KCGSKDNIGGC,
(SEQ ID NO: 803)
CGSKDNIKGGC,
(SEQ ID NO: 804)
GSKDNIKHGGC,
(SEQ ID NO: 805)
SKDNIKIVGGC,
(SEQ ID NO: 806)
KDNIKHVPGGC,
and
(SEQ ID NO: 807)
DNIKHVPGGGC.

23. The peptide of claim 19, wherein the peptide comprises an amino acid sequence selected from the group consisting of
(SEQ ID NO: 815)
VTSKCGSLGGC,
(SEQ ID NO: 816)
TSKCGSLGGGC,
(SEQ ID NO: 817)
SKCGSLGNGGC,
(SEQ ID NO: 818)
KCGSLGNIGGC,
(SEQ ID NO: 819)
CGSLGNIHGGC,
(SEQ ID NO: 820)
GSLGNIHHGGC,
(SEQ ID NO: 821)
SLGNIHHKGGC,
(SEQ ID NO: 822)
LGNIHHKPGGC,
and
(SEQ ID NO: 823)
GNIHHKPGGGC.

24. The peptide of claim 20, wherein the peptide comprises an amino acid sequence selected from the group consisting of
(SEQ ID NO: 831)
RVQSKIGSGGC,
(SEQ ID NO: 832)
VQSKIGSLGGC,
(SEQ ID NO: 833)
QSKIGSLDGGC,
(SEQ ID NO: 834)
SKIGSLDNGGC,
(SEQ ID NO: 835)
KIGSLDNIGGC,
(SEQ ID NO: 836)
IGSLDNITGGC,
(SEQ ID NO: 837)
GSLDNITHGGC,
(SEQ ID NO: 838)
SLDNITHVGGC,
(SEQ ID NO: 839)
LDNITHVPGGC,
and
(SEQ ID NO: 840)
DNITHVPGGGC.

25. The peptide of claim 21, comprising an amino acid sequence of KSKIGSTEGGC (SEQ ID NO:778).

26. The peptide of claim 21, comprising an amino acid sequence of SKIGSTENGGC (SEQ ID NO:779).

27. The peptide of claim 21, comprising an amino acid sequence of TENLKHQPGGC (SEQ ID NO:784).

28. The peptide of claim 21, comprising an amino acid sequence of ENLKHQPGGGC (SEQ ID NO:785).

29. The peptide of claim 11, wherein the peptide comprises an amino acid sequence selected from the group consisting of
(SEQ ID NO: 851)
VKSKIGSTEGGGC,
(SEQ ID NO: 852)
PDLKNVKSGGGC,
(SEQ ID NO: 853)
DLKNVKSKGGGC,
(SEQ ID NO: 854)
LKNVKSKIGGGC,
(SEQ ID NO: 855)
KNVKSKIGGGGC,
(SEQ ID NO: 856)
NVKSKIGSGGGC,
(SEQ ID NO: 843)
VKSKIGSTEGGGC,
(SEQ ID NO: 844)
KSKIGSTEGGGC,
(SEQ ID NO: 845)
SKIGSTENGGGC,
(SEQ ID NO: 846)
KIGSTENLGGGG,
(SEQ ID NO: 847)
IGSTENLKGGGC,
(SEQ ID NO: 848)
GSTENLKHGGGG,
(SEQ ID NO: 849)
STENLKHQGGGC,
(SEQ ID NO: 850)
TENLKHOPGGGC,
(SEQ ID NO: 857)
ENLKHQPGGGGC,
(SEQ ID NO: 858)
NLKHQPGGGGGC,
and
(SEQ ID NO: 859)
LKHQPGGGGGGC.

30. The peptide of claim 11, wherein the peptide comprises an amino acid sequence selected from the group consisting of
(SEQ ID NO: 860)
LDLSNVQSGGGC,
(SEQ ID NO: 861)
DLSNVQSKGGGC,
(SEQ ID NO: 862)
LSNVQSKCGGGC,
(SEQ ID NO: 863)
SNVQSKCGGGGC,
(SEQ ID NO: 864)
NVQSKCGSGGGC,
(SEQ ID NO: 865)
VQSKCGSKGGGC,
(SEQ ID NO: 866)
QSKCGSKDGGGC,
(SEQ ID NO: 867)
SKCGSKDNGGGC,
(SEQ ID NO: 868)
KCGSKDNIGGGC,
(SEQ ID NO: 869)
CGSKDNIKGGGC,
(SEQ ID NO: 870)
GSKDNIKHGGGC,
(SEQ ID NO: 871)
SKDNIKHVGGGC,
(SEQ ID NO: 872)
KDNIKHVPGGGC,
(SEQ ID NO: 873)
DNIKHVPGGGGC,
(SEQ ID NO: 874)
NIKHVPGGGGGC,
and
(SEQ ID NO: 875)
IKHVPGGGGGGC.

31. The peptide of claim 11, wherein the peptide comprises an amino acid sequence selected from the group consisting of
(SEQ ID NO: 876)
VDLSKVTSGGGC,
(SEQ ID NO: 877)
DLSKVTSKGGGC,
(SEQ ID NO: 878)
LSKVTSKCGGGC,
(SEQ ID NO: 879)
SKVTSKCGGGGC,
(SEQ ID NO: 880)
KVTSKCGSGGGC,
(SEQ ID NO: 881)
VTSKCGSLGGGC,
(SEQ ID NO: 882)
TSKCGSLGGGGC,
(SEQ ID NO: 883)
SKCGSLGNGGGC,
(SEQ ID NO: 884)
KCGSLGNIGGGC,
(SEQ ID NO: 885)
CGSLGNIHGGGC,
(SEQ ID NO: 886)
GSLGNIHHGGGC,
(SEQ ID NO: 887)
SLGNIHHKGGGC,
(SEQ ID NO: 888)
LGNIHHKPGGGC,
(SEQ ID NO: 889)
GNIHHKPGGGGC,
(SEQ ID NO: 890)
NIHHKPGGGGGC,
and
(SEQ ID NO: 891)
IHHKPGGGGGGC.

32. The peptide of claim 11, wherein the peptide comprises an amino acid sequence selected from the group consisting of
(SEQ ID NO: 892)
LDFKDRVQGGGC,
(SEQ ID NO: 893)
DFKDRVQSGGGC,
(SEQ ID NO: 894)
FKDRVQSKGGGC,
(SEQ ID NO: 895)
KDRVQSKIGGGC,
(SEQ ID NO: 896)
DRVQSKIGGGGC,
(SEQ ID NO: 897)
RVQSKIGSGGGC,
(SEQ ID NO: 898)
VQSKIGSLGGGC,
(SEQ ID NO: 899)
QSKIGSLDGGGC,
(SEQ ID NO: 900)
SKIGSLDNGGGC,
(SEQ ID NO: 901)
KIGSLDNIGGGC,
(SEQ ID NO: 902)
IGSLDNITGGGC,
(SEQ ID NO: 903)
GSLDNITHGGGC,
(SEQ ID NO: 904)
SLDNITHVGGGC,
(SEQ ID NO: 905)
LDNITHVPGGGC,
(SEQ ID NO: 906)
DNITHVPGGGGC,
(SEQ ID NO: 907)
NITHVPGGGGGC,
and
(SEQ ID NO: 908)
ITHVPGGGGGGC.

33. The peptide of claim 11, wherein the peptide comprises an amino acid sequence selected from the group consisting of
(SEQ ID NO: 966)
CGGGSKIGSTDNIKH,
(SEQ ID NO: 967)
CGGGSKIGSKDNIKH,
(SEQ ID NO: 968)
CGGGSKIGSLDNIKH,
(SEQ ID NO: 963)
CGGSKIGSTDNIKH,
(SEQ ID NO: 964)
CGGSKIGSKDNIKH,
(SEQ ID NO: 965)
CGGSKIGSLDNIKH.

34. The peptide of claim 29, wherein the peptide comprises an amino acid sequence selected from the group consisting of
(SEQ ID NO: 843)
VKSKIGSTEGGGC,
(SEQ ID NO: s44)
KSKIGSTEGGGC,
(SEQ ID NO: 845)
SKIGSTENGGGC,
(SEQ ID NO: 846)
KIGSTENLGGGC,
(SEQ ID NO: 847)
IGSTENLKGGGC,
(SEQ ID NO: 848)
GSTENLKHGGGC,
(SEQ ID NO: 849)
STENLKHQGGGC,
(SEQ ID NO: 850)
TENLKHQPGGGC,
and
(SEQ ID NO: 857)
ENLKHQPGGGGC.

35. The peptide of claim 30, wherein the peptide comprises an amino acid sequence selected from the group consisting of
(SEQ ID NO: 865)
VQSKOGSKGGGC,
(SEQ ID NO: 866)
QSKCGSKDGGGC,
(SEQ ID NO: 867)
SKCGSKDNGGGC,
(SEQ ID NO: 868)
KCGSKDNIGGGC,
(SEQ ID NO: 869)
CGSKDNIKGGGC,
(SEQ ID NO: 870)
GSKDNIKHGGGC,
(SEQ ID NO: 871)
SKDNIKHVGGGC,
(SEQ ID NO: 872)
KDNIKHVPGGGC,
and
(SEQ ID NO: 873)
DNIKHVPGGGGC.

36. The peptide of claim 31, wherein the peptide comprises an amino acid sequence selected from the group consisting of
(SEQ ID NO: 881)
VTSKCGSLGGGC,
(SEQ ID NO: 882)
TSKCGSLGGGGC,
(SEQ ID NO: 883)
SKOGSLGNGGGC,
(SEQ ID NO: 884)
KCGSLGNIGGGG,
(SEQ ID NO: 885)
CGSLGNIHGGGC,
(SEQ ID NO: 886)
GSLGNIHHGGGC,
(SEQ ID NO: 887)
SLGNIHHKGGGC,
(SEQ ID NO: 888)
LGNIHHKPGGGC,
and
(SEQ ID NO: 889)
GNIHHKPGGGGC.

37. The peptide of claim 32, wherein the peptide comprises an amino acid sequence selected from the group consisting of
(SEQ ID NO: 897)
RVQSKIGSGGGC,
(SEQ ID NO: 898)
VQSKIGSLGGGC,
(SEQ ID NO: 899)
QSKIGSLDGGGC,
(SEQ ID NO: 890)
SKIGSLDNGGGC,
(SEQ ID NO: 891)
KIGSLDNIGGGC,
(SEQ ID NO: 892)
IGSLDNITGGGC,
(SEQ ID NO: 893)
GSLDNITHGGGC,
(SEQ ID NO: 894)
SLDNITHVGGGC,
(SEQ ID NO: 895)
LDNITHVPGGGC,
(SEQ ID NO: 896)
DNITHVPGGGGC.

38. The peptide of claim 33, comprising an amino acid sequence of CGGGSKIGSKDNIKH (SEQ ID NO:967).

39. The peptide of claim 33, comprising an amino acid sequence of CGGSKIGSKDNIKH (SEQ ID NO:964).

40. The peptide of claim 34, comprising an amino acid sequence of VKSKIGSTEGGGC (SEQ ID NO:843).

41. The peptide of claim 34, comprising an amino acid sequence of KSKIGSTEGGGC (SEQ ID NO:844).

42. The peptide of claim 34, comprising an amino acid sequence of SKIGSTENGGGC (SEQ ID NO:845).

43. The peptide of claim 34, comprising an amino acid sequence of KIGSTENLGGGC (SEQ ID NO:846).

44. The peptide of claim 34, comprising an amino acid sequence of IGSTENLKGGGC (SEQ ID NO:847).

45. The peptide of claim 34, comprising an amino acid sequence of GSTENLKHGGGC (SEQ ID NO:848).

46. The peptide of claim 34, comprising an amino acid sequence of STENLKHQGGGC (SEQ ID NO:849).

47. The peptide of claim 34, comprising an amino acid sequence of TENLKHQPGGGC (SEQ ID NO:850).

48. The peptide of claim 34, comprising an amino acid sequence of ENLKHQPGGGGC (SEQ ID NO:857).

49. The peptide of claim 13, comprising an amino acid sequence of QIINK (SEQ ID NO:17).

50. The peptide of claim 13, comprising an amino acid sequence of QIVYKPV (SEQ ID NO:02).

51. The peptide of claim 13, comprising an amino acid sequence of NIKHVP (SEQ ID NO:04).

52. The peptide of claim 13, comprising an amino acid sequence of NIKHVPG (SEQ ID NO:05).

53. The peptide of claim 14, comprising an amino acid sequence of EIVYKSV (SEQ ID NO:21).

54. The peptide of claim 16, comprising an amino acid sequence of VKSKIGSTE (SEQ ID NO:589).

55. The peptide of claim 16, comprising an amino acid sequence of KSKIGSTE (SEQ ID NO:590).

56. The peptide of claim 16, comprising an amino acid sequence of SKIGSTEN (SEQ ID NO:598).

57. The peptide of claim 16, comprising an amino acid sequence of KIGSTENL (SEQ ID NO:605).

58. The peptide of claim 16, comprising an amino acid sequence of IGSTENLK (SEQ ID NO:611).

59. The peptide of claim 16, comprising an amino acid sequence of GSTENLKH (SEQ ID NO:616).

60. The peptide of claim 16, comprising an amino acid sequence of STENLKHQ (SEQ ID NO:620).

61. The peptide of claim 16, comprising an amino acid sequence of TENLKHQP (SEQ ID NO:476).

61. The peptide of claim 16, comprising an amino acid sequence of ENLKHQPG (SEQ ID NO:470).

62. A peptide comprising 5-13 amino acids from residues 244-400 of SEQ ID NO:01 or from residues 1-150 of SEQ ID NO:750, comprising at least one amino acid substitution.

63. The peptide of claim 62, comprising an amino acid sequence of SKIGSTENLKH (SEQ ID NO:909)

64. The peptide of claim 63, wherein one of the at least one amino acid substitutions comprises an isoleucine substitution for a lysine at position 10.

65. The peptide of either one of claim 63 or 64, wherein one of the at least one amino acid substitutions comprises a lysine or leucine substitution for a tyrosine at position 6.

66. The peptide of any one of claims 63-65, wherein one of the at least one amino acid substitutions comprises an aspartic acid or glycine substitution for a glutamic acid at position 7.

67. The peptide of claim 62, wherein the peptide comprises an amino acid sequence selected from the group consisting of
(SEQ ID NO: 909)
SKIGSTENLKH,
(SEQ ID NO: 910)
SKIGSTENIKH,
(SEQ ID NO: 911)
SKIGSKDNLKH,
(SEQ ID NO: 912)
SKIGSKENIKH,
(SEQ ID NO: 913)
SKIGSLENLKH,
(SEQ ID NO: 914)
SKIGSLENIKH,
(SEQ ID NO: 915)
SKIGSTDNLKH,
(SEQ ID NO: 916)
SKIGSTDNIKH,
(SEQ ID NO: 917)
SKIGSKDNLKH,
(SEQ ID NO: 918)
SKIGSKDNIKH,
(SEQ ID NO: 919)
SKIGSLDNLKH,
(SEQ ID NO: 920)
SKIGSLDNIKH,
(SEQ ID NO: 921)
SKIGSTGNLKH,
(SEQ ID NO: 922)
SKIGSTGNIKH,
(SEQ ID NO: 923)
SKIGSKGNLKH,
(SEQ ID NO: 924)
SKIGSKGNIKH,
(SEQ ID NO: 925)
SKIGSLGNLKH,
(SEQ ID NO: 926)
SKIGSLGNIKH,

68. The peptide of claim 67, wherein the peptide comprises an amino acid sequence selected from the group consisting of SKIGSTDNIKH (SEQ ID NO:916), SKIGSKDNIKH (SEQ ID NO:918), or SKIGSLDNIKH (SEQ ID NO:920).

69. The peptide of any one of claims 62-68, wherein the peptide further comprises a C-terminal cysteine (—C), -GGC or -GGGC or an N-terminal cysteine (C—), CGG- or CGGG-.

70. The peptide of claim 69, wherein the peptide comprises an amino acid sequence selected from the group consisting of
(SEQ ID NO: 927)
SKIGSTENLKHGGC,
(SEQ ID NO: 928)
SKIGSTENIKHGGC,
(SEQ ID NO: 929)
SKIGSKDNLKHGGC,
(SEQ ID NO: 930)
SKIGSKENIKHGGC,
(SEQ ID NO: 931)
SKIGSLENLKHGGC,
(SEQ ID NO: 932)
SKIGSLENIKHGGC,
(SEQ ID NO: 933)
SKIGSTDNLKHGGC,
(SEQ ID NO: 934)
SKIGSTDNIKHGGC,
(SEQ ID NO: 935)
SKIGSKDNLKHGGC,
(SEQ ID NO: 936)
SKIGSKDNIKHGGC,
(SEQ ID NO: 937)
SKIGSLDNLKHGGC,
(SEQ ID NO: 938)
SKIGSLDNIKHGGC,
(SEQ ID NO: 939)
SKIGSTGNLKHGGC,
(SEQ ID NO: 940)
SKIGSTGNIKHGGC,
(SEQ ID NO: 941)
SKIGSKGNLKHGGC,
(SEQ ID NO: 942)
SKIGSKGNIKHGGC,
(SEQ ID NO: 943)
SKIGSLGNLKHGGC,
(SEQ ID NO: 944)
SKIGSLGNIKHGGC,
(SEQ ID NO: 945)
SKIGSTENLKHGGGC,
(SEQ ID NO: 946)
SKIGSTENIKHGGGC,
(SEQ ID NO: 947)
SKIGSKDNLKHGGGC,
(SEQ ID NO: 948)
SKIGSKENIKHGGGC,
(SEQ ID NO: 949)
SKIGSLENLKHGGGC,
(SEQ ID NO: 950)
SKIGSLENIKHGGGC,
(SEQ ID NO: 951)
SKIGSTDNLKHGGGC,
(SEQ ID NO: 952)
SKIGSTDNIKHGGGC,
(SEQ ID NO: 953)
SKIGSKDNLKHGGGC,
(SEQ ID NO: 954)
SKIGSKDNIKHGGGC,
(SEQ ID NO: 955)
SKIGSLDNLKHGGGC,
(SEQ ID NO: 956)
SKIGSLDNIKHGGGC,
(SEQ ID NO: 957)
SKIGSTGNLKHGGGC,
(SEQ ID NO: 958)
SKIGSTGNIKHGGGC,
(SEQ ID NO: 959)
SKIGSKGNLKHGGGC,
(SEQ ID NO: 961)
SKIGSKGNIKHGGGC,
(SEQ ID NO: 960)
SKIGSLGNLKHGGGC,
(SEQ ID NO: 962)
SKIGSLGNIKHGGGC,
(SEQ ID NO: 963)
CGGSKIGSTDNIKH,
(SEQ ID NO: 964)
CGGSKIGSKDNIKH,
(SEQ ID NO: 965)
CGGSKIGSLDNIKH,
(SEQ ID NO: 966)
CGGGSKIGSTDNIKH,
(SEQ ID NO: 967)
CGGGSKIGSKDNIKH,
and
(SEQ ID NO: 968)
CGGGSKIGSLDNIKH.

71. The peptide of any one of claims 1-70, further comprising a linker to a carrier at a C-terminal portion of the peptide or at a N-terminal portion of the peptide.

72. The peptide of claim 71, wherein the linker comprises an amino acid sequence.

73. The peptide of claim 72, wherein the linker amino acid sequence comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids or one (1) amino acid.

74. The peptide of claim 72, wherein the linker comprises an amino acid sequence selected from the group consisting of AA, AAA, KK, KKK, SS, SSS AGAG, GG, GGG, GAGA, KGKG, (GGS)n, (GGGS)n, and (GGGGS)n, where n=1-3.

75. The peptide of any one of claims 1 to 74, wherein the peptide or linker to the carrier, if present, further comprises a C-terminal cysteine (C).

76. The peptide of any one of claims 1 to 75, wherein the peptide further comprises a blocked amine at the N-terminus.

77. An immunotherapy composition, comprising one or more of the peptides of any of claims 1 to 76.

78. The immunotherapy composition of claim 77, wherein the one or more peptides further comprises a linker to a carrier at a C-terminal portion of the peptide.

79. The immunotherapy composition of claim 78, wherein the linker to the carrier comprises an amino acid sequence selected from the group consisting of AA, AAA, KK, KKK, SS, SSS AGAG, GG, GGG, GAGA, KGKG, (GGS)n, (GGGS)n, and (GGGGS)n, where n=1-3.

80. The immunotherapy composition of either one of claim 78 or 79, wherein the carrier comprises serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

81. The immunotherapy composition of claim 80, wherein the carrier is CRM197.

82. The immunotherapy composition of claim 81, wherein the carrier is diphtheria toxoid.

83. The immunotherapy composition of any one of claims 77 to 82, further comprising at least one pharmaceutically acceptable diluent.

84. The immunotherapy composition of any one of claims 77 to 83, further comprising a multiple antigen presenting system (MAP).

85. The immunotherapy composition of claim 84, wherein the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

86. A pharmaceutical composition comprising (a) one or more of the peptides of any of claims 1 to 76 or (b) the immunotherapy composition of any of claims 77 to 85 and at least one adjuvant.

87. The pharmaceutical composition of claim 86, wherein the adjuvant is selected from the group consisting of aluminum hydroxide, aluminum phosphate, aluminum sulfate, 3 De-O-acylated monophosphoryl lipid A (MPL), QS-21, TQL-1055, QS-18, QS-17, QS-7, Complete Freund's Adjuvant (CFA), Incomplete Freund's Adjuvant (1FA), oil in water emulsions (such as squalene or peanut oil), CpG, polyglutamic acid, polylysine, AddaVax™, MF59®, and combinations thereof.

88. The pharmaceutical composition of claim 87, wherein the adjuvant is QS-21 or TQL-1055.

89. The pharmaceutical composition of claim 87, wherein the adjuvant is MPL.

90. The pharmaceutical composition of claim 87, wherein the adjuvant is a combination of MPL and QS-21 or a combination of MPL and TQL-1055.

91. The pharmaceutical composition of any of claims 86 to 90, wherein the adjuvant comprises a liposomal formulation.

92. The pharmaceutical composition of any of claims 86 to 91, wherein the composition comprises at least one pharmaceutically acceptable diluent.

93. The pharmaceutical formulation of any of claims 86 to 92, comprising a multiple antigen presenting system (MAP).

94. The pharmaceutical formulation of claim 93, wherein the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

95. A nucleic acid comprising a nucleic acid sequence encoding a peptide of any one of claims 1 to 76 or the immunotherapy composition of claims 77 to 85.

96. A nucleic acid immunotherapy composition comprising the nucleic acid of claim 95 and at least one adjuvant.

97. A method of treating or effecting prophylaxis of Alzheimer's disease in a subject, comprising administrating to the subject the immunotherapy composition of any of claims 77 to 85 or the pharmaceutical compositions of any of claims 86 to 94.

98. A method of inhibiting or reducing aggregation of tau in a subject having or at risk of developing Alzheimer's disease, comprising, administering to the subject the immunotherapy composition of any of claims 77 to 85 or the pharmaceutical formulations of any of claims 86 to 94.

99. A method of treating or effecting prophylaxis of Alzheimer's disease in a subject, comprising administrating to the subject the nucleic acid immunotherapy composition of claim 95 or 96.

100. A method of inhibiting or reducing aggregation of tau in a subject having or at risk of developing Alzheimer's disease, comprising administering to the subject the nucleic acid immunotherapy composition of claim 96 or 96.

101. The method of any of claims 97 to 100, further comprising repeating the administering at least a second time, at least a third time, at least a fourth time, at least a fifth time, or at least a sixth time.

102. The method of claim 101, further comprising repeating the administering at an interval of about 21 to about 28 days.

103. A method of inducing an immune response in an animal, comprising administering to the animal any one of the peptide of claims 1 to 76, the immunotherapy composition of claims 77 to 85, the pharmaceutical formulations of claims 86 to 94 or the nucleic acid immunotherapy composition of claims 95 to 96 in a regimen effective to generate an immune response comprising antibodies that specifically bind to tau.

104. The method of claim 103, wherein the immune response comprises antibodies that specifically bind to tau.

105. The method of any of claims 103 to 104, wherein the inducing the immune response comprises antibodies that specifically bind to the microtubule region of tau.

106. An immunization kit comprising the immunotherapy composition of any of claims 77 to 85.

107. The kit of claim 106, further comprising an adjuvant.

108. The kit of claim 107, wherein the immunotherapy composition is in a first container and the adjuvant is in a second container.

109. A kit comprising the nucleic acid immunotherapy composition of claim 96.

110. The kit of claim 109, further comprising an adjuvant.

111. The kit of claim 110, wherein the nucleic acid is in a first container and the adjuvant is in a second container.
